(19) United States
(12) Patent Application Publication
(10) Pub. No.: US 2024/0366655 A1
(43) Pub. Date: Nov. 7, 2024

(54) COMBINATION DRUG THERAPIES

(71) Applicant: CYBIN IRL LIMITED, DUBLIN (IE)

(72) Inventors: MICHAEL PALFREYMAN, ST. PETERSBURG, FL (US); ALEX NIVOROZHKIN, WEST ROXBURY, MA (US); BRETT J. GREENE, FITCHBURG, MA (US); ROBERT MINO, JUPITER, FL (US)

(73) Assignee: CYBIN IRL LIMITED, DUBLIN (IE)

(21) Appl. No.: 18/688,125

(22) PCT Filed: Mar. 31, 2022
(86) PCT No.: PCT/EP2022/058574

§ 371 Date: Feb. 29, 2024

Related U.S. Application Data
(60) Provisional application No. 63/241,891, filed on Sep. 8, 2021.

(51) Int. Cl. A61K 33/00; A61K 31/135; A61K 31/4045; A61P 25/22

(52) U.S. Cl. CPC A61K 33/00; A61K 31/135; A61K 31/4045; A61P 25/22

(57) ABSTRACT

Combination drag therapies comprising a 5-HTZA receptor agonist and an N-methyl-D-aspartate (NMDA) receptor antagonist (e.g., nitrous oxide, ketamine, etc.) are provided. Also described are pharmaceutical compositions and methods of treating a central nervous system (CNS) disorder or a psychiatric disease using the combination drug therapy, for example, via aerosol inhalation.

### CROSS REFERENCE TO RELATED APPLICATIONS

This application claims the benefit of U.S. Provisional Application No. 63/241,891 filed Sep. 8, 2021, which is incorporated herein by reference in its entirety.

### FIELD

The present disclosure relates to combination drug therapies, specifically combination drug therapies that include a 5-HT_{2A} receptor agonist and an N-methyl-D-aspartate (NMDA) receptor antagonist, a pharmaceutical composition containing the combination drug therapies, as well as methods of treating diseases or conditions therewith, including central nervous system (CNS) disorders or psychiatric disorders.

### BACKGROUND

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Mood disorders such as depression are ubiquitous mental illnesses. Therapies for such disorders were initially discovered in the 1940s, including first-generation drugs such as monoamine oxidase inhibitors. These drugs were followed by tricyclic antidepressants and later the development of second-generation of antidepressants, selective serotonin reuptake inhibitors and serotonin-norepinephrine reuptake inhibitors. The latter revolutionized the treatment of depression, and to this day remain a staple of therapy. However, current therapies can take weeks or months to reach full effectiveness after treatment commencement, and less than 50% of patients show a response to such drugs.

Emerging strategies for the treatment of central nervous system (CNS) diseases have focused on serotonin (5-HT) receptor subfamily 5-HT₂ receptor agonists as well as glutamate N-methyl-D-aspartate (NMDA) receptor antagonists, through the action of psychedelic compounds such as psilocybin, psilocin, N,N-dimethyltryptamine (DMT), phenethylamines, 5-methoxy-N,N-dimethyltryptamine (5-MeO-DMT), lysergic acid diethylamide (LSD), and ketamine. Such serotonin 5-HT₂ receptor agonists and glutamate N-methyl-D-aspartate (NMDA) receptor antagonist, which are used to affect serotonin and glutamate pathways, respectively, have shown promising results in early-stage clinical trials and clinic settings. These receptors are believed to be important for the treatment and pathologies of depression, schizophrenia, anxieties and a number of other mental disorders. As an example, (S)-ketamine (Spravato®) has recently been approved for treating suicidal ideations and for treatment-resistant depression (TRD) when taken in conjunction with an oral (conventional) antidepressant. Psilocybin is currently in phase 2 clinical trials for TRD and major depressive disorder (MDD).

Psychedelics are named such because of their experiential effects on the user. Most often, the psychedelic experience acts to enhance the mood of the user when consumed. However, administration of psychedelics can evoke a negative experience for the patient, presenting as acute psychedelic crisis, colloquially known as a “bad trip,” in which the patient experiences feelings of remorse or distress, or other symptoms such as agitation, confusion, intense anxiety, and psychotic episodes, which may be transient or extended in nature. It is believed that overstimulation of the 5-HT_{2A} receptors elevates the risk of a bad trip experience. Bad trip experiences can cause an interruption of therapy, a discontinuation of therapy, or even an adverse therapy event.

In the clinical setting, the medical professional, therapeutic monitor, or other session participant in the supervised psychedelic experience may try to reduce acute psychedelic crisis events through pre-disposing the patient to positive thinking or lowered anxiety through reassurance or other professional psychological means. If the acute psychedelic crisis rises to a significant level, the medical professional overseeing the psychedelic experience may administer benzodiazepines or other anxiolytics. Unfortunately, this administration may be counter-active of the desired therapeutic outcome of the administration of the psychedelic. The challenges are exacerbated in populations being treated for general anxiety disorder, social anxiety disorder, forms of depression, or alcohol use disorder or other disorders of addiction, as these conditions are tied to increased psychological stress factors and therefore pose an increased risk of acute psychedelic crisis.

Further, NMDA receptor antagonists are dissociative anesthetics with a wide range of effects in humans. At high doses (e.g., anesthetic and sub-anesthetic doses), significant numbers of patients experience adverse psychiatric symptoms including dissociative effects, e.g., out of body experience, dissociation of the mind from the body, distorted perception, and hallucination.

### SUMMARY

In view of the forgoing, there is a need for new psychedelic therapies with robust therapeutic efficacy that minimize psychiatric adverse effects.

Accordingly, it is one object of the present disclosure to provide novel combination drug therapies that meet these criteria.

It is another object of the present disclosure to provide novel pharmaceutical compositions for delivering the combination drug therapies of the present disclosure.

It is yet another object of the present disclosure to provide novel methods of treating a central nervous system (CNS) disorder or a psychiatric disease with the combination drug therapies of the present disclosure to a subject in need thereof.

These and other objects, which will become apparent during the following detailed description, have been achieved by the inventors' discovery that a combination of a 5-HT_{2A} (serotonin) receptor agonist and an N-methyl-D-aspartate (NMDA) receptor antagonist unexpectedly yields combination drug therapies that exhibit beneficial therapeutic activities by regulating both serotonin and glutamate uptakes while improving patient experience such as, for example, through increased safety and/or decreased acute psychedelic crisis. In particular, the combination of the 5-HT_{2A} receptor agonist and NMDA receptor antagonist promotes patient experience by providing therapeutic benefit while reducing or eliminating psychiatric adverse effects such as acute psychedelic crisis and dissociative effects, which may be caused by taking the 5-HT_{2A} receptor agonist or the NMDA receptor antagonist alone.

Thus, the present disclosure provides:
(1) A combination drug therapy, comprising:
a 5-HT_{2A} receptor agonist; and
a N-methyl-D-aspartate (NMDA) receptor antagonist.
(2) The combination drug therapy of (1), wherein the 5-HT_{2A} receptor agonist is a tryptamine derivative.
(3) The combination drug therapy of (2), wherein the tryptamine derivative is at least one selected from the group consisting of psilocybin, psilocin, N,N-dimethyltryptamine (DMT), 5-methoxy-N,N-dimethyltryptamine (5-MeO-DMT), 2-(1H-indol-3-yl)-N,N-bis(methyl-d)ethan-1-amine-1,1,2,2-d₄ (DMT-d₁₀), and 2-(5-methoxy-1H-indol-3-yl)-N,N-bis(methyl-d₃)ethan-1-amine-1,1,2,2d₄ (5-MeO-DMT-d₁₀), or a pharmaceutically acceptable salt or solvate thereof.
(4) The combination drug therapy of (2) or (3), wherein the tryptamine derivative is at least one selected from the group consisting of N,N-dimethyltryptamine (DMT), 5-methoxy-N,N-dimethyltryptamine (5-MeO-DMT), 2-(1H-indol-3-yl)-N,N-bis(methyl-d₃)ethan-1-amine-1,1,2,2-d₄ (DMT-d₁₀), and 2-(5-methoxy-1H-indol-3-yl)-N,N-bis(methyl-d₃)ethan-1-amine-1,1,2,2-d₄ (5-MeO-DMT-d₁₀), or a pharmaceutically acceptable salt or solvate thereof.
(5) The combination drug therapy of (1), wherein the 5-HT_{2A} receptor agonist is a phenethylamine derivative.
(6) The combination drug therapy of (5), wherein the phenethylamine derivative is at least one selected from the group consisting of 3,4-methylenedioxymethamphetamine (MDMA) and 2,5-dimethoxy-4-bromophenethylamine (2C-B), or a pharmaceutically acceptable salt, stereoisomer, or solvate thereof.
(7) The combination drug therapy of any one of more of (1) to (6), wherein the 5-HT_{2A} receptor agonist is a tryptamine derivative comprising at least one deuterium atom or a phenethylamine derivative comprising at least one deuterium atom.
(8) The combination drug therapy of any one of more of (1) to (7), wherein the NMDA receptor antagonist is at least one selected from the group consisting of ketamine, nitrous oxide, memantine, and dextromethorphan, or a pharmaceutically acceptable salt, stereoisomer, or solvate thereof.
(9) The combination drug therapy of any one of more of (1) to (8), wherein the NMDA receptor antagonist is nitrous oxide.
(10) The combination drug therapy of (1), wherein the 5-HT_{2A} receptor agonist is N,N-dimethyltryptamine (DMT) or a pharmaceutically acceptable salt or solvate thereof, and the NMDA receptor antagonist is nitrous oxide.
(11) The combination drug therapy of (1), wherein the 5-HT_{2A} receptor agonist is 5-methoxy-N,N-dimethyltryptamine (5-MeO-DMT) or a pharmaceutically acceptable salt or solvate thereof, and the NMDA receptor antagonist is nitrous oxide.
(12) The combination drug therapy of (1), wherein the 5-HT_{2A} receptor agonist is 2-(1H-indol-3-yl)-N,N-bis(methyl-d₃)ethan-1-amine-1,1,2,2-d₄ (DMT-d₁₀) or a pharmaceutically acceptable salt or solvate thereof, and the NMDA receptor antagonist is nitrous oxide.
(13) The combination drug therapy of (1), wherein the 5-HT_{2A} receptor agonist is 2-(5-methoxy-1H-indol-3-yl)-N,N-bis(methyl-d₃)ethan-1-amine-1,1,2,2-d₄ (5-MeO-DMT-d₁₀) or a pharmaceutically acceptable salt or solvate thereof, and the NMDA receptor antagonist is nitrous oxide.
(14) The combination drug therapy of any one of more of (1) to (8), wherein the NMDA receptor antagonist is ketamine, or a pharmaceutically acceptable salt, stereoisomer, or solvate thereof.
(15) The combination drug therapy of (1), wherein the 5-HT_{2A} receptor agonist is N,N-dimethyltryptamine (DMT) or a pharmaceutically acceptable salt or solvate thereof, and the NMDA receptor antagonist is ketamine or a pharmaceutically acceptable salt, stereoisomer, or solvate thereof.
(16) The combination drug therapy of (1), wherein the 5-HT_{2A} receptor agonist is 5-methoxy-N,N-dimethyltryptamine (5-MeO-DMT) or a pharmaceutically acceptable salt or solvate thereof, and the NMDA receptor antagonist is ketamine or a pharmaceutically acceptable salt, stereoisomer, or solvate thereof.
(17) The combination drug therapy of (1), wherein the 5-HT_{2A} receptor agonist is 2-(1H-indol-3-yl)-N,N-bis(methyl-d₃)ethan-1-amine-1,1,2,2-d₄ (DMT-d₁₀) or a pharmaceutically acceptable salt or solvate thereof, and the NMDA receptor antagonist is ketamine or a pharmaceutically acceptable salt, stereoisomer, or solvate thereof.
(18) The combination drug therapy of (1), wherein the 5-HT_{2A} receptor agonist is 2-(5-methoxy-1H-indol-3-yl)-N,N-bis(methyl-d₃)ethan-1-amine-1,1,2,2-d₄ (5-MeO-DMT-d₁₀) or a pharmaceutically acceptable salt or solvate thereof, and the NMDA receptor antagonist is ketamine or a pharmaceutically acceptable salt, stereoisomer, or solvate thereof.
(19) The combination drug therapy of any one of more of (1) to (18), wherein the 5-HT_{2A} receptor agonist and the NMDA receptor antagonist are combined into a single pharmaceutical composition.
(20) The combination drug therapy of (19), wherein the 5-HT_{2A} receptor agonist and the NMDA receptor antagonist are combined into an aerosol for administration via inhalation.
(21) The combination drug therapy of (20), wherein the aerosol is in the form of a mist.
(22) The combination drug therapy of (20) or (21), wherein the aerosol comprises the 5-HT_{2A} receptor agonist dissolved in a liquid phase of the aerosol.
(23) The combination drug therapy of any one of more of (20) to (22), wherein the aerosol comprises the NMDA receptor antagonist in a gas phase of the aerosol.
(24) The combination drug therapy of (19), wherein the 5-HT_{2A} receptor agonist and the NMDA receptor antagonist are combined in a transdermal patch.
(25) The combination drug therapy of any one of more of (1) to (18), wherein the 5-HT_{2A} receptor agonist and the NMDA receptor antagonist are provided as separate pharmaceutical compositions.
(26) The combination drug therapy of (25), wherein the 5-HT_{2A} receptor agonist is provided as an intravenous injection, and the NMDA receptor antagonist is provided as a therapeutic gas mixture.
(27) The combination drug therapy of (25), wherein the 5-HT_{2A} receptor agonist is provided as an aerosol, and the NMDA receptor antagonist is provided as a therapeutic gas mixture.
(28) The combination drug therapy of any one of more of (25) to (27), wherein the separate pharmaceutical compositions are administered sequentially (29) The combination drug therapy of any one of ore of (25) to (27), wherein the separate pharmaceutical compositions are administered concurrently.
(30) A pharmaceutical composition, comprising the combination drug therapy of any one of more of (1) to (24) and a pharmaceutically acceptable excipient.
(31) The pharmaceutical composition of (30), which is formulated for administration via inhalation.
(32) A method of treating a subject with a central nervous system (CNS) disorder or a psychiatric disease, the method comprising:
administering to the subject a therapeutically effective amount of a 5-HT_{2A} receptor agonist and a N-methyl-D-aspartate (NMDA) receptor antagonist.
(33) The method of (32), wherein the CNS disorder or a psychiatric disease is at least one selected from the group consisting of post-traumatic stress disorder (PTSD), major depressive disorder (MDD), treatment-resistant depression (TRD), suicidal ideation, suicidal behavior, major depressive disorder with suicidal ideation or suicidal behavior, melancholic depression, atypical depression, dysthymia, non-suicidal self-injury disorder (NSSID), bipolar and related disorders, obsessive-compulsive disorder (OCD), compulsive behavior and other related symptoms, generalized anxiety disorder (GAD), acute psychedelic crisis, social anxiety disorder, alcohol use disorder, opioid use disorder, amphetamine use disorder, nicotine use disorder, cocaine use disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, chronic fatigue syndrome, Lyme disease, gambling disorder, anorexia nervosa, bulimia nervosa, binge-eating disorder, pedophilic disorder, exhibitionistic disorder, voyeuristic disorder, fetishistic disorder, sexual masochism or sadism disorder, transvestic disorder, sexual dysfunction, peripheral neuropathy, and obesity.
(34) The method of (32) or (33), wherein the CNS disorder or a psychiatric disease is major depressive disorder (MDD).
(35) The method of (32) or (33), wherein the CNS disorder or a psychiatric disease is treatment-resistant depression (s).
(36) The method of (32) or (33), wherein the CNS disorder or a psychiatric disease is generalized anxiety disorder (GAD).
(37) The method of (32) or (33), wherein the CNS disorder or a psychiatric disease is social anxiety disorder.
(38) The method of (32) or (33), wherein t u CNS disorder or a psychiatric disease is obsessive-compulsive disorder (OCD).
(39) The method of (32) or (33), wherein the CNS disorder or a psychiatric disease is alcohol use disorder.
(40) The method of any one or more of (32) to (39), wherein the 5-HT_{2A} receptor agonist is administered at a dose of about 0.01 mg/kg to about 3 mg/kg.
(41) The method of any one or more of (32) to (40), wherein the 5-HT_{2A} receptor agonist and the NMDA receptor antagonist are administered 1 to 8 times over a treatment course.
(42) The method of any one or more of (32) to (41), wherein the 5-HT_{2A} receptor agonist and the NMDA receptor antagonist are administered concurrently as a single pharmaceutical composition.
(43) The method of any one or more of (32) to (42), wherein the 5-HT_{2A} receptor agonist and the NMDA receptor antagonist are administered as an aerosol to the patient by inhalation.
(44) The method of (43), wherein the aerosol is in the form of a mist.
(45) The method of (43) or (44), wherein the aerosol comprises the 5-HT_{2A} receptor agonist dissolved in a liquid phase of the aerosol.
(46) The method of any one or more of (43) to (45), wherein the aerosol is prepared by nebulization of the 5-HT_{2A} receptor agonist.
(47) The method of (46), wherein the nebulization is performed with a device selected from the group consisting of a jet nebulizer, an ultrasonic nebulizer, a breath-actuated nebulizer, and a vibrating mesh nebulizer.
(48) The method of any one or more of (32) to (47), wherein the NMDA receptor antagonist is nitrous oxide.
(49) The method of (48), wherein the aerosol comprises the nitrous oxide in a gas phase of the aerosol.
(50) The method of (49), wherein the gas phase of the aerosol comprises a therapeutic gas mixture comprising the nitrous oxide.
(51) The method of (50), wherein the therapeutic gas mixture is a mixture of nitrous oxide and O₂, a mixture of N₂O and air, a mixture of N₂O and medical air, a mixture of N₂O, N₂, and O₂, a mixture of N₂O O₂ enriched medical air, or a mixture of N₂O, He, and O₂.
(52) The method of (50) or (51), wherein the nitrous oxide is present in the therapeutic as mixture at a concentration of 5 to 50 vol %, relative to a total volume of the therapeutic gas mixture.
(53) The method of any one or more of (50) to (52), wherein the therapeutic gas mixture acts as a driving gas for generation of the aerosol.
(54) The method of any one or more of (50) to (53), wherein the therapeutic gas mixture acts as a carrier gas for generation of the aerosol.
(55) The method of any one or more of (43) to (54), wherein the aerosol is administered for 20 to 60 minutes.
(56) The method of any one or more of (32) to (55), wherein the 5-HT_{2A} receptor agonist is N,N-dimethyltryptamine (DMT) or a pharmaceutically acceptable salt or solvate thereof, and the NMDA receptor antagonist is nitrous oxide.
(57) The method of any one or more of (32) to (55), wherein the 5-HT_{2A} receptor agonist is 5-methoxy-N,N-dimethyltryptamine (5-MeO-DMT) or a pharmaceutically acceptable salt or solvate thereof, and the NMDA receptor antagonist is nitrous oxide.
(58) The method of any one or more of (32) to (55), wherein the 5-HT_{2A} receptor agonist is 2-(1H-indol-3-yl)-N-bis(methyl-)eth-1-amine-1,1,2,2-d₄ (DMT-d₁₀) or a pharmaceutically acceptable salt or solvate thereof, and the NMDA receptor antagonist is nitrous oxide.
(59) The method of any one or more of (32) to (55), wherein the 5-HT_{2A} receptor agonist is 2-(5-methoxy-1H-indol-3-yl)-N,N-bis(methyl-d₃)ethan-1-amine-1,1,2,2-d₄(5-MeO-DMT-d₁₀) or a pharmaceutically acceptable salt or solvate thereof, and the NMDA receptor antagonist is nitrous oxide.
(60) The method of any one or more of (32) to (42), wherein the 5-HT_{2A} receptor agonist and the NMDA receptor antagonist are administered transdermally to the patient via a transdermal patch.
(61) The method of (60), wherein the 5-HT_{2A} receptor agonist is N,N-dimethyltryptamine (DMT) or a pharmaceutically acceptable salt or solvate thereof, and the NMDA receptor antagonist is ketamine or a pharmaceutically acceptable salt, stereoisomer, or solvate thereof.
(62) The method of (60), wherein the 5-HT_{2A} receptor agonist is 5-methoxy-N,N-dimethyltryptamine (5-MeO-DMT) or a pharmaceutically acceptable salt or solvate thereof, and the NMDA receptor antagonist is ketamine or a pharmaceutically acceptable salt, stereoisomer, or solvate thereof.
(63) The method of (60), wherein the 5-HT_{2A} receptor agonist is 2-(1H-indol-3-yl)-N,N-bis(methyl-d₃)ethan-1-amine-1,1,2,2-d₄ (DMT-d₁₀) or a pharmaceutically acceptable salt or solvate thereof, and the NMDA receptor antagonist is ketamine or a pharmaceutically acceptable salt, stereoisomer, or solvate thereof.
(64) The method of (60), wherein the 5-HT_{2A} receptor agonist is 2-(5-methoxy-1H-indol-3-yl)-N,N-bis(methyl-d₃)ethan-1-amine-1,1,2,2-d₄(5-MeO-DMT-d₁₀) or a pharmaceutically acceptable salt or solvate thereof, and the NMDA receptor antagonist is ketamine or a pharmaceutically acceptable salt, stereoisomer, or solvate thereof.
(65) The method of any one or more of (32) to (41), wherein the 5-HT_{2A} receptor agonist and the NMDA receptor antagonist are administered sequentially as separate pharmaceutical compositions.
(66) The method of any one or more of (32) to (41), wherein the 5-HT_{2A} receptor agonist and the NMDA receptor antagonist are administered concurrently as separate pharmaceutical compositions.
(67) The method of any one or more of (32) to (41) or (65) or (66), wherein the 5-HT_{2A} receptor agonist is administered intravenously and the NMDA receptor antagonist is administered via inhalation.
(68) The method of (67), wherein the 5-HT_{2A} receptor agonist is administered to the subject intravenously as a single bolus.
(69) The method of (68), wherein the 5-HT_{2A} receptor agonist is administered at a dose of about 0.1 mg/kg to about 0.8 mg/kg.
(70) The method of (67), wherein the 5-HT_{2A} receptor agonist is administered to the subject intravenously as a perfusion.
(71) The method of (70), wherein the 5-HT_{2A} receptor agonist is administered at a dose of about 0.1 mg/kg to about 0.8 mg/kg.
(72) The method of (70) or (71), wherein the perfusion is administered over a duration of about 5 minutes to about 1 hour.
(73) The method of any one or more of (67) to (72), wherein the 5-HT_{2A} receptor agonist is administered to the subject intravenously as a bolus followed by a perfusion.
(74) The method of (73), wherein a dose of the 5-HT_{2A} receptor agonist administered via the bolus and the perfusion are each independently in a range from about 0.1 mg/kg to about 0.8 mg/kg.
(75) The method of any one or more of (32) to (41) or (65) to (74), wherein the NMDA antagonist is nitrous oxide, which is administered via inhalation as a therapeutic gas mixture comprising the nitrous oxide.
(76) The method of (75), wherein the therapeutic gas mixture is a mixture of nitrous oxide and O₂, a mixture of N₂O and air, a mixture of N₂O and medical air, a mixture of N₂O, N₂, and O₂, a mixture of N₂O O₂ enriched medical air, or a mixture of N₂O, He, and O₂.
(77) The method of (75) or (76), wherein the nitrous oxide is present in the therapeutic gas mixture at a concentration of 5 to 50 vol %, relative to a total volume of the therapeutic gas mixture.
(78) The method of any one or more of (32) to (55), (60), or (65) to (77), wherein the 5-HT_{2A} receptor agonist is N,N-dimethyltryptamine (DMT) or a pharmaceutically acceptable salt or solvate thereof.
(79) The method of any one or more of (32) to (55), (60), or (65) to (77), wherein the 5-HT_{2A} receptor agonist is 5-methoxy-N,N-dimethyltryptamine (5-MeO-DMT) or a pharmaceutically acceptable salt or solvate thereof.
(80) The method of any one or more of (32) to (55), (60), or (65) to (77), wherein the 5-HT_{2A} receptor agonist is 2-(1H-indol-3-yl)-N,N-bis(methyl-d₃)ethan-1-amine-1,1,2,2-d₄ (DMT-d₁₀) or a pharmaceutically acceptable salt or solvate thereof.
(81) The method of any one or more of (32) to (55), (60), or (65) to (77), wherein the 5-HT_{2A} receptor agonist is 2-(5-methoxy-1H-indol-3-yl)-N,N-bis(methyl-d₃)ethan-1-amine-1,1,2,2-d₄ (5-MeO-DMT-d₁₀) or a pharmaceutically acceptable salt or solvate thereof.
(82) The method of any one or more of (32) to (81), wherein the 5-HT_{2A} receptor agonist and the NMDA receptor antagonist are administered in amounts effective to reduce or inhibit acute psychedelic crisis.
(83) The method of any one or more of (32) to (82), wherein the 5-HT_{2A} receptor agonist and the NMDA receptor antagonist are administered in amounts effective to reduce or inhibit dissociative effects.
(84) An inhalation delivery device for delivery of a combination of nitrous oxide and a 5-HT_{2A} receptor agonist by inhalation to a patient in need thereof, comprising:
an inhalation outlet portal for administration of the combination of nitrous oxide and the 5-HT_{2A} receptor agonist to the patient;
a container configured to deliver nitrous oxide to the inhalation outlet portal; and a device configured to generate and deliver an aerosol comprising the 5-HT_{2A} receptor agonist to the inhalation outlet portal.
(85) The inhalation delivery device of (84), wherein the inhalation outlet portal is a mouthpiece or a mask covering the patient's nose and mouth.
(86) The inhalation delivery device of (84) or (85), wherein the device configured to generate and deliver the aerosol to the inhalation outlet portal is a nebulizer.
(87) The inhalation delivery device of (86), wherein the nebulizer is a jet nebulizer and the nitrous oxide acts as a driving gas for the jet nebulizer.
(88) The inhalation delivery device of any one or more of (84) to (87), further comprising electronics configured to provide remote activation and operational control of the inhalation delivery device.
(89) A fast-acting therapeutic combination, comprising:
a 5-HT_{2A} receptor agonist having an elimination half-life of up to 2 hours; and nitrous oxide.
(90) The fast-acting therapeutic combination of (89), wherein the elimination half-life of the 5-HT_{2A} receptor agonist is less than 30 minutes.
(91) The fast-acting therapeutic combination of (89) or (90), wherein the 5-HT_{2A} receptor agonist N,N-dimethyltryptamine (DMT), 5-methoxy-N,N-dimethyltryptamine (5-MeO-DMT), 2-(1H-indol-3-yl)-N,N-bis(methyl-d₅)ethan-1-amine-1,1,2,2-d₄ (DMT-d₁₀), or 2-(5-methoxy-1H-indol-3-yl)-N,N-bis(methyl-d₃)ethan-1-amine-1,1,2,2-d₄ (5-MeO-DMT-d₁₀).
(92) A rescue medicine kit comprising the fast-acting therapeutic combination of any one or more of (89) to (91), in one or more containers packaged separately or together.
(93) A method treating a subject with an acute psychiatric condition, the method comprising:
administering to the subject a therapeutically effective amount of the fast-acting therapeutic combination of any one or more of (89) to (91) for a time period of less than or equal to the elimination half-life of the 5-HT_{2A} receptor agonist.

### BRIEF DESCRIPTION OF THE DRAWINGS

The forgoing paragraphs have been provided by way of general introduction and are not intended to limit the scope of the following claims. The described embodiments, together with further advantages, will be best understood by reference to the following detailed description when considered in conjunction with the accompanying drawings, wherein:

FIGS. 1A-1B show a directed flow exposure chamber housed within a secondary containment chamber (top view; FIG. 1A) and a depiction of rats held in restraining tubes with their snouts protruding from the ends of the restraining tubes into the exposure chambers (FIG. 1B);

FIG. 2 shows DMT and DMT-d₁₀ plasma concentration-time profiles after IV administration (1 mg/kg) in rats;

FIG. 3 shows DMT and DMT-d₁₀ plasma concentration-time profiles after inhalation administration (14.7 mg/kg and 15.3 mg/kg, respectively) in rats;

FIG. 4 shows DMT and DMT-d₁₀ plasma concentration-time profiles after PO (oral gavage; OG) administration (10 mg/kg) in rats;

FIG. 5 shows DMT plasma concentration-time profiles after IV, inhalation, and PO (OG) administration, with doses normalized to 1 mg/kg;

FIG. 6 shows DMT-d₁₀ plasma concentration-time profiles after IV, inhalation, and PO (OG) administration, with doses normalized to 1 mg/kg;

FIG. 7 illustrates a transparent air-tight plexiglass anesthetic induction chamber setup for pre-clinical rodent studies; and

FIG. 8 shows a general experimental design for a human study probing synergistic interactions of DMT with nitrous oxide (N₂O).

### DETAILED DESCRIPTION

In the following detailed description, it is understood that other embodiments may be utilized and structural and operational changes may be made without departure from the scope of the present embodiments disclosed herein.

### Definitions

Unless defined otherwise, all technical and scientific terms used herein have the same meaning as is commonly understood by one of skill in the art to which this disclosure belongs.

“Alkyl” refers to monovalent saturated aliphatic hydrocarbyl groups having from 1 to 10 carbon atoms and such as 1 to 6 carbon atoms, or 1 to 5, or 1 to 4, or 1 to 3, or 1 to 2 carbon atoms.

This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl (t-Bu)((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 10 substituents selected from the group consisting of deuterium, alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂— alkyl, —SO₂-aryl, —SO₂-heteroaryl, and —NR′R″, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups having from 1 to 6, including, for example, 1 to 3 carbon atoms that are either straight-chained or branched, and which are optionally interrupted with one or more groups selected from —O—, —NR¹⁰—, —NR¹⁰C(O), —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms, for example 2 to 4 carbon atoms and having at least 1, for example from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms, for example, 2 to 3 carbon atoms and having at least 1 and for example, from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O) “Acylamino” refers to the groups —NR²⁰C(O)alkyl, —NR²⁰C(O)substituted alkyl, NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹²², wherein R² and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R^{2′} and R² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²¹R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂— substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O— alkynyl, —O—C(O)O-substituted alkynyl, —O—C(O)O-aryl, —O—C(O)O-substituted aryl, —O—C(O)O— cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclohexyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from deuterium, alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and for example, from 1 to 2 double bonds.

The term “substituted cycloalkenyl” refers to cycloalkenyl groups having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO— substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, benzo[d][1,3]oxathiole, benzo[d][1,3]dioxole, and the like.

Unless otherwise constrained by the definition for the heterocyclic substituent, such heterocyclic groups can be optionally substituted with 1 to 5, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaninoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂— substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (=S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, =NR⁷⁰, ═N—OR⁷⁰, ═N₂ or ═S) on saturated carbon atoms in the specified group or radical are, unless otherwise specified, deuterium, —R⁶⁰, halo, ═O, —OR⁷⁰, —SR⁷⁰, —NR⁸⁰R⁸⁰, trihalomethyl, —CN, —OCN, —SCN, —NO, —NO₂, =N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of 0, N and S, of which N may have —H or C₁-C₃ alkyl substitution; and each M⁺ is a counter ion with a net single positive charge. Each M may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the disclosure and the other a typical counter ion such as chloride, or two ionized compounds disclosed herein can serve as counter ions for such divalent alkali earth ions, or a doubly ionized compound of the disclosure can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, deuterium, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃-2(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻, —OCO₂R⁷⁰, —OC(S)OR⁷⁰, —NR⁷⁰C(O)R⁷⁰, —NR⁷⁰C(S)R⁷⁰, —NR⁷⁰CO₂⁻NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —O C(S)R⁷⁰, —OC(O)OR⁷⁰, —OC(S)OR⁷⁰, —NR⁷⁰C(O)R⁷⁰, —NR⁷C(S)R⁷⁰, —NR⁷⁰C(O)OR⁷⁰, —NR⁷⁰C(S) OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M are as previously defined.

In addition to the disclosure herein, in some embodiments, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein, unless specified otherwise. In such cases, the maximum number of such substitutions is three. For example, serial substitutions of substituted aryl groups specifically contemplated herein are limited to substituted aryl-(substituted aryl)-substituted aryl. However, substituent groups defined as e.g., polyethers may contain serial substitution greater than three, e.g., —O—(CH₂CH₂O)ₙ—H, where n can be 1, 2, 3, or greater.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

When it is defined that a substituent or group “comprise(s) deuterium,” it is to be understood that the substituent or group may itself be deuterium, or the substituent or group may contain at least one deuterium substitution in its chemical structure. For example, when substituent “—R” is defined to “comprise(s) deuterium,” it is to be understood that —R may be —D (-deuterium), or a group such as —CD₃ that is consistent with the other requirements set forth of —R.

The phrases “pharmaceutically acceptable,” “physiologically acceptable,” and the like, are employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. When referencing salts, the phrases “pharmaceutically acceptable salt,” “physiologically acceptable salt,” and the like, means a salt which is acceptable for administration to a patient, such as a mammal (salts with counterions having acceptable mammalian safety for a given dosage regime). As is well known in the art, such salts can be derived from pharmaceutically acceptable inorganic or organic bases, by way of example, sodium, potassium, calcium, magnesium, ammonium, and tetraalkylammonium salts, and the like, and when the molecule contains a basic functionality, addition salts with inorganic acids, such as hydrochloride, hydrobromide, sulfate, sulfamate, phosphate, nitrate, perchlorate salts, and the like, and addition salts with organic acids, such as formate, tartrate, besylate, mesylate, acetate, maleate, oxalate, fumarate, benzoate, salicylate, succinate, oxalate, glycolate, hemi-oxalate, hemi-fumarate, propionate, stearate, lactate, citrate, ascorbate, pamoate, hydroxymaleate, phenylacetate, glutamate, 2-acetoxybenzoate, tosylate, ethanedisulfonate, isethionate salts, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a physical association of a compound or salt of the present disclosure with one or more solvent molecules, whether organic, inorganic, or a mixture of both. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Some examples of solvents include, but are not limited to, methanol, ethanol, isopropanol, N,N-dimethylformamide, tetrahydrofuran, dimethylsulfoxide, and water. When the solvent is water, the solvate formed is a hydrate (e.g., monohydrate, dihydrate, etc.). Exemplary solvates thus include, but are not limited to, hydrates, methanolates, ethanolates, isopropanolates, etc. Methods of solvation are generally known in the art.

“Stereoisomer” and “stereoisomers” refer to compounds that have same atomic connectivity but different atomic arrangement in space. Stereoisomers include cis-trans isomers, E and Z isomers, enantiomers, and diastereomers. All forms such as racemates and optically pure stereoisomers of the compounds are contemplated herein. Chemical formulas and compounds which possess at least one stereogenic center, but are drawn without reference to stereochemistry, are intended to encompass both the racemic compound, as well as the separate stereoisomers, e.g., R- and/or S-stereoisomers, each permutation of diastereomers so long as those diastereomers are geometrically feasible, etc.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the compounds herein can exist in different salt, solvate, and stereoisomer forms, and the present disclosure is intended to include all permutations of salts, solvates and stereoisomers, such as a solvate of a pharmaceutically acceptable salt of a stereoisomer of subject compound.

A “vapor” is a solid substance in the gas phase at a temperature lower than its critical temperature, meaning that the vapor can be condensed to a liquid by increasing the pressure on it without reducing the temperature.

An “aerosol”, as used herein, is a suspension of fine solid particles or liquid droplets in a gas phase (e.g., air, oxygen, helium, nitrous oxide, and other gases, as well as mixtures thereof). A “mist”, as used herein, is a subset of aerosols, differing from a vapor, and is a dispersion of liquid droplets (liquid phase) suspended in the gas phase (e.g., air, oxygen, helium, and mixtures thereof). The liquid droplets of an aerosol or mist can comprise a drug moiety dissolved in an aqueous liquid, organic solvent, or a mixture thereof. The gas phase of an aerosol or mist can comprise air, oxygen, helium, or other gases such as nitrous oxide, including mixtures thereof. Mists do not comprise solid particulates. Aerosols and mists of the present disclosure can be generated by any suitable methods and devices, examples of which are set forth herein, e.g., through use of an inhaler or nebulizer.

As used herein, the language “sustained-release” or “controlled-release” describes the release period for certain formulations of the present disclosure formulated to increase the release period e.g., to a maximum value, which is ultimately limited by the time the gastrointestinal tract naturally excretes all drugs with food. As used herein, the language “release period” describes the time window in which any active ingredient described herein is released from the excipient (e.g., matrix) to afford plasma concentrations of active ingredient(s) described herein. The start time of the release period is defined from the point of oral administration to a subject, which when ingested orally is considered nearly equivalent to entry into the stomach, and initial dissolution by gastric enzymes and acid. The end time of the release period is defined as the point when the entire loaded drug is released. In some embodiments, the release period can be greater than about 4 hours, 8 hours, 12 hours, 16 hours, or 20 hours, greater than or equal to about 24 hours, 28 hours, 32 hours, 36 hours, or 48 hours, or less than about 48 hours, 36 hours, 4 hours or less, 3 hours or less, 2 hours or less, or 1 hour or less.

The term “stable,” “stability,” and the like, as used herein includes chemical stability and solid state (physical) stability. The term “chemical stability” means that the compound can be stored in anisolated form, or in the form of a formulation in which it is provided in admixture with for example, pharmaceutically acceptable carriers, diluents or adjuvants as described herein, under normal storage conditions, with little or no chemical degradation or decomposition. “Solid-state stability” means the compound can be stored in an isolated solid form, or the form of a solid formulation in which it is provided in admixture with, for example, pharmaceutically acceptable carriers, diluents or adjuvants as described herein, under normal storage conditions, with little or no solid-state transformation (e.g., hydration, dehydration, solvatization, desolvatization, crystallization, recrystallization or solid-state phase transition).

As used herein, the term “composition” is equivalent to the term “formulation.”

The terms “administer”, “administering”, “administration”, and the like, as used herein, refer to the methods that may be used to enable delivery of the active ingredient(s) and/or the composition to the desired site of biological action. Routes or modes of administration are as set forth herein.

As used herein, “concurrent” administration or administration performed “concurrently” refers to administration of two or more active ingredients at the same time (e.g., simultaneously, in unison, such as the case when administered within the same dosage form); at overlapping times (e.g., where a first active ingredient is administered continually over a period of time, such as continually over 20 minutes, and a second active ingredient is administered at some point within or overlapping with the time period of administration of the first active ingredient); or at times which are non-overlapping but are nearly abutting, i.e., are separated by no more than 30 seconds, i.e., where the start of administration of a first active ingredient is separated from the end time of administration of a second active ingredient, or vice versa, by no more than 30 seconds. For example, administration of two injections, one immediately following the other within 30 seconds, is considered to be concurrent administration herein. “Sequential” administration or administration performed “sequentially” refers to administration of two or more active ingredients with an interval of time between their non-overlapping end points of greater than 30 seconds (i.e., where the start of administration of a first active ingredient is separated from the end time of administration of a second active ingredient, or vice versa, by more than 30 seconds).

As used herein, the term “inhalation session” describes a dosing event whereby the subject inhales a given dose of drug, irrespective of the number of breadths needed to inhale the given dose. For example, a subject prescribed to take 10 mg of a drug twice a day would undertake two inhalation sessions, each inhalation session providing 10 mg of the drug. The length of time and the number of breaths for each inhalation session would be dependent on factors such as the inhalation device used, the amount of drug that is drawn per breath, the concentration of the drug in the dosage form, the subject's breathing pattern, etc.

The term “treating” or “treatment” as used herein means the treating or treatment of a disease or medical condition in a patient, such as a mammal (particularly a human) that includes: ameliorating the disease or medical condition, such as, eliminating or causing regression of the disease or medical condition in a patient; suppressing the disease or medical condition, for example by, slowing or arresting the development of the disease or medical condition in a patient; or alleviating a symptom of the disease or medical condition in a patient. A treatment can provide a therapeutic benefit such as the eradication or amelioration of one or more of the physiological or psychological symptoms associated with the underlying condition, disease, or disorder such that an improvement is observed in the patient, notwithstanding the fact that the patient may still be affected by the condition. In some embodiments, treatment may refer to prophylaxis, i.e., preventing the disease or medical condition from occurring or otherwise delaying the onset of the disease or medical condition in a patient.

A “patient” or “subject,” used interchangeably herein, can be any mammal including, for example, a human. A patient or subject can have a condition to be treated or can be susceptible to a condition to be treated.

As used herein, and unless otherwise specified, the terms “inhibit,” and “inhibiting” refer to the inhibition of the onset, recurrence or spread of a disease, disorder, or condition, or of one or more symptoms thereof. The terms encompass the prevention or reduction of a symptom of the particular disease, disorder, or condition. Subjects with familial history of a disease, disorder, or condition, in particular, are candidates for preventive regimens in some embodiments. In addition, subjects who have a history of recurring symptoms are also potential candidates for the prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease, disorder, or condition, or of one or more symptoms thereof. Often, the beneficial effects that a subject derives from a prophylactic and/or therapeutic agent do not result in a cure of the disease, disorder, or condition. In this regard, the term “managing” encompasses treating a subject who had suffered from the particular disease, disorder, or condition in an attempt to prevent or minimize the recurrence of the disease, disorder, or condition.

“Therapeutically effective amount” refers to an amount of a compound(s) sufficient to treat a specified disorder or disease or one or more of its symptoms and/or to prevent the occurrence of the disease or disorder (prophylactically effective amount). As used herein, and unless otherwise specified, a “prophylactically effective amount” of an active ingredient(s), is an amount sufficient to prevent a disease, disorder, or condition, or prevent its recurrence. The term “prophylactically effective amount” can encompass an amount that improves overall prophylaxis or enhances the prophylactic efficacy of another prophylactic agent.

The term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”. A “continuous” administration schedule means administration every day without interruption during the treatment course. If the administration schedule follows an “intermittent” administration schedule, then days of administration may be followed by “rest days” or days of non-administration of drug within the course. A “drug holiday” indicates that the drug is not administered in a predetermined administration schedule. For example, after undergoing several courses of treatment, a subject may be prescribed a regulated drug holiday as part of the administration schedule, e.g., prior to re-recommencing active treatment.

The language “toxic spikes” is used herein to describe spikes in concentration of any compound described herein that would produce side-effects of sedation or psychotomimetic effects, e.g., hallucination, dizziness, and nausea; which can not only have immediate repercussions, but also influence treatment compliance. In particular, side effects may become more pronounced at blood concentration levels above about 300 ng/L (e.g. above about 300, 400, 500, 600 or more ng/L).

As used herein, and unless otherwise specified, a “neuropsychiatric disease or disorder” is a behavioral or psychological problem associated with a known neurological condition, and typically defined as a cluster of symptoms that co-exist. Examples of neuropsychiatric disorders include, but are not limited to, schizophrenia, cognitive deficits in schizophrenia, attention deficit disorder, attention deficit hyperactivity disorder, bipolar and manic disorders, depression or any combinations thereof.

“Inflammatory conditions” or “inflammatory disease,” as used herein, refers broadly to chronic or acute inflammatory diseases. Inflammatory conditions and inflammatory diseases, include but are not limited to rheumatic diseases (e.g., rheumatoid arthritis, osteoarthritis, psoriatic arthritis) spondyloarthropathies (e.g., ankylosing spondylitis, reactive arthritis, Reiter's syndrome), crystal arthropathies (e.g., gout, pseudogout, calcium pyrophosphate deposition disease), multiple sclerosis, Lyme disease, polymyalgia rheumatica; connective tissue diseases (e.g., systemic lupus erythematosus, systemic sclerosis, polymyositis, dermatomyositis, Sjogren's syndrome); vasculitides (e.g., polyarteritis nodosa, Wegener's granulomatosis, Churg-Strauss syndrome); inflammatory conditions including consequences of trauma or ischaemia, sarcoidosis; vascular diseases including atherosclerotic vascular disease, atherosclerosis, and vascular occlusive disease (e.g., atherosclerosis, ischaemic heart disease, myocardial infarction, stroke, peripheral vascular disease), and vascular stent restenosis; ocular diseases including uveitis, corneal disease, iritis, iridocyclitis, and cataracts.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value may vary up or down by 5%. For example, for a value of about 100, means 95 to 105 (or any value between 95 and 105).

### Combination Drug Therapies

The present disclosure is directed to combination drug therapies based on administration of both a 5-HT_{2A} receptor agonist and a N-methyl-D-aspartate (NMDA) receptor antagonist as active ingredients. The co-action of such a combination can provide numerous benefits including, but not limited to, 1) improved efficacy and duration of response, 2) faster onset of action, 3) reduced systemic toxicity, 4) reduced neurotoxicity, and 5) enhanced patient experience by inducing a euphoric psychedelic event thereby reducing or eliminating psychiatric adverse effects such as acute psychedelic crisis (bad trip) and dissociative effects from hallucinogens (out of body experience) regularly seen when taking the 5-HT_{2A} receptor agonist or the NMDA receptor antagonist alone.

### 5-HT_{2A} Receptor Agonists

As used herein, a “5-HT_{2A} receptor agonist” refers to a compound that increases the activity of a 5-HT_{2A} receptor, which is a subtype of the 5-HT₂ receptor that belongs to the serotonin receptor family, including both partial and full agonists. Non-limiting examples of such agonists include, but are not limited to, a tryptamine derivative and a phenethylamine derivative. The 5-HT_{2A} receptor agonist used in the combination drug therapy may be a single compound, or a mixture of compounds, e.g., a mixture of tryptamine derivatives, a mixture of phenethylamine derivative, or a mixture of one or more tryptamine derivatives and one or more phenethylamine derivatives, including pharmaceutically acceptable salts, stereoisomers, solvates, or prodrugs thereof.

Examples of tryptamine derivatives include, but are not limited to, psilocybin (3-[2-(dimethylamino)ethyl]-1H-indol-4-yl dihydrogen phosphate) and derivatives thereof, e.g., psilocin (4-hydroxy-N,N-dimethyltryptamine), N-desmethyl-psilocybin (3-[2-(methylamino)ethyl]-1H-indol-4-yl dihydrogen phosphate), 4-HO-NMT (4-hydroxy-N-methyltryptamine), norbaeocystin ([3-(2-aminoethyl)-1H-indol-4-yl] dihydrogen phosphate, 4-hydroxytryptamine, 3-[2-(N,N,N-trimethylamino)ethyl]-1H-indol-4-yl dihydrogen phosphate salts, and 4-hydroxy TMT salts (salts of 4-hydroxy-N,N,N-trimethyltryptamine); N,N-dimethyltryptamine (DMT); 5-hydroxy-N,N-dimethyltryptamine (5-OH-DMT); 5-methoxy-N,N-dimethyltryptamine (5-MeO-DMT); lysergic acid diethylamide (LSD) (a complex tryptamine) and derivatives thereof, e.g., LA-SS-Az (“LSZ” or (2S,4S)-1-[[(8β)-9,10-Didehydro-6-(methyl)ergolin-8-yl]carbonyl]-2,4-dimethylazetidine); ibogaine (a complex tryptamine); or deuterated analogs thereof, e.g., DMT-d₁₀(2-(1H-indol-3-yl)-N,N-bis(methy-d₃)ethan-1-amine-1,1,2,2-d₄), 5-MeO-DMT-d₁₀ (2-(5-methoxy-1H-indol-3-yl)-N,N-bis(methyl-d₃)ethan-1-amine-1,1,2,2-d₄), etc.; as well as pharmaceutically acceptable salts, solvates, or stereoisomers thereof.

In some embodiments, the 5-HT_{2A} receptor agonist is a tryptamine derivative, which is a compound of Formula (I), Formula (II), Formula (II-a), Formula (II-b), Formula (II-c), or Formula (II-d), which will be described hereinafter, or a pharmaceutically acceptable salt, solvate, or stereoisomer thereof, or a combination thereof.

In preferred embodiments, the 5-HT_{2A} receptor agonist is at least one tryptamine derivative selected from the group consisting of psilocin, psilocybin, N,N-dimethyltryptamine (DMT), 5-hydroxy-N,N-dimethyltryptamine (5-OH-DMT), 5-methoxy-N,N-dimethylryptamine (5-MeO-DMT), DMT-d₁₀ (2-(1H-indol-3-yl)-N,N-bis(methyl-d₃)ethan-1-amine-1,1,2,2-d₄), and 5-MeO-DMT-d₁₀ (2-(5-methoxy-1H-indol-3-yl)-N,N-bis(methyl-d₃)ethan-1-amine-1,1,2,2-d₄), or a pharmaceutically acceptable salt or solvate thereof.

Examples of phenethylamine derivatives include, but are not limited to, 3,4-methylenedioxymethamphetamine (MDMA); 2C-X phenethylamines such as 2,5-dimethoxy-4-bromophenethylamine (2C-B), (4-chloro-2,5-dimethoxyphenethyl)amine (2C-C), 2,5-dimethoxy-4-methylphenethylamine (2C-D); 3,4-methylenedioxy-N-ethylamphetamine (MDEA); 1,3-benzodioxolyl-N-methylbutanamine (MBDB); trimethoxyamphetamines (TMAs) such as 3,4,5-trimethoxyamphetamine (TMA), 2,4,5-trimethoxy-amphetamine (TMA-2), 2,3,4-trimethoxyamphetamine (TMA-3), 2,3,5-trimethoxyamphetamine (TMA-4), 2,3,6-trimethoxyamphetamine (TMA-5), and 2,4,6-trimethoxyamphetamine (TMA-6); trimethoxyphenethylamines such as 3,4,5-trinethoxyphenethylamine (mescaline) and isomescaline (2,3,4-trimethoxyphenethylamine); 2,5-dimethoxy-4-methylamphetamine (DOM); 2,5-dimethoxy-4-ethylamphetamine (DOET); 1-(2,5-dimethoxyphenyl)-2-aminopropane; 2,5-dimethoxy-4-iodoamphetamine (DOI), including (R)-DOI; 4-chloro-2,5-dimethoxy-amphetamine (DOC); 4-bromo-2,5-dimethoxy-amphetamine (DOB); 4-bromo-2,5-dimethoxy-methamphetamine (MDOB); and 4-bromo-3,6-dimethoxybenzocyclobuten-1-yl) methylamine (2C-BCB); or deuterated analogs thereof; as well as pharmaceutically acceptable salts, solvate, or stereoisomers thereof.

In some embodiments, the 5-HT_{2A} receptor agonist is a phenethylamine derivative, which is a compound of Formula (III), Formula (III-a), Formula (IV), Formula (IV-a), Formula (IV-b), Formula (V), Formula (V-a), Formula (V-b), Formula (VI), Formula (VI-a), Formula (VI-b), which will be described hereinafter, or a pharmaceutically acceptable salt, solvate, or stereoisomer thereof, or a combination thereof.

In preferred embodiments, the 5-HT_{2A} receptor agonist is at least one phenethylamine derivative selected from the group consisting of 3,4-methylenedioxymethamphetamine (MDMA), and 2,5-dimethoxy-4-bromophenethylamine (2C-B), or a pharmaceutically acceptable salt, solvate, or stereoisomer thereof.

The 5-HT_{2A} receptor agonist used herein may be a compound having at least one deuterium atom. For example, the 5-HT_{2A} receptor agonist may be a tryptamine derivative of the following Formula (I), Formula (II), Formula (II-a), Formula (II-b), Formula (II-c), Formula (II-d), comprising at least one deuterium atom, or a combination thereof. Alternatively, or additionally, the 5-HT_{2A} receptor agonist may be a phenethylamine derivative of the following Formula (III), or Formula (III-a), an N-substituted phenethylamine (NSP) of the following Formula (IV), Formula (IV-a), Formula (IV-b), Formula (V), Formula (V-a), Formula (V-b), Formula (VI), Formula (VI-a), Formula (VI-b), comprising at least one deuterium atom, or a combination thereof.

In some embodiments, the 5-HT_{2A} receptor agonist is a compound of Formula (I), or a pharmaceutically acceptable salt, stereoisomer, solvate, or prodrug thereof

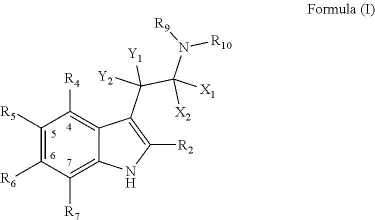

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, and unsubstituted or substituted alkoxy;
R₆ and R₇ are independently selected from the group consisting of hydrogen, deuterium, and halogen; and
R₉ and R₁₀ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl.

X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ and X₂ are different. In some embodiments, X₁ is hydrogen or deuterium, and X₂ is a substituted or unsubstituted C₁-C₆ alkyl. In some embodiments, X₂ is an unsubstituted C₁-C₆ alkyl, examples of which include, but are not limited to, methyl, ethyl, and n-propyl, preferably methyl. In some embodiments, X₂ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X₁ and X₂ is deuterium while the other is hydrogen. In some embodiments, one or more of X₁ and X₂ is a substituted or unsubstituted C₃-C₁₀ cycloalkyl, some embodiments, one or more of X₁ and X₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, one or more of X₁ and X₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, X₁ and/or X₂ is an unsubstituted or substituted alkenyl, e.g., a unsubstituted or substituted allyl.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, one of Y₁ and Y₂ is deuterium while the other is hydrogen.

In some embodiments, R₂ is deuterium. In some embodiments, R₂ is hydrogen. In some embodiments, R₂ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₂ is a substituted C₁-C₆ alkyl. When R₂ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₂ is a substituted or unsubstituted C₃-C₁₀ cycloalkyl. In some embodiments, R₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₂ is an unsubstituted or substituted alkenyl, e.g., a unsubstituted or substituted allyl.

R₄ and R₅ may be the same, or different. In some embodiments, R₄ is deuterium. In some embodiments, R₄ is hydrogen. In some embodiments, R₄ is hydroxy. In some embodiments, R₄ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₄ is a substituted alkoxy. When R₄ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be-OCDH₂, —OCD₂H, —OCD₃, —OCFH₂, —OCF₂H, —OCF₃, etc.

In some embodiments, R₅ is deuterium. In some embodiments, R₅ is hydrogen. In some embodiments, R₅ is hydroxy. In some embodiments, R₅ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₅ is a substituted alkoxy. When R₅ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be-OCDH₂, —OCD₂H, —OCD₃, —OCFH₂, —OCF₂H, —OCF₃, etc.

R₆ and R₇ may be the same, or different. R₆ and R₇ may be, independently, hydrogen, deuterium, or a halogen for example —Br, —F, —Cl, or —I.

R₉ and R₁₀ may be the same, or different. In some embodiments, R₉ and R₁₀ are the same. In some embodiments, R₉ and R₁₀ are hydrogen. In some embodiments, R₉ and R₁₀ are different. In some embodiments, R₉ is hydrogen, and R₁₀ is a substituted or unsubstituted C₁-C₆ alkyl. In some embodiments, R₁₀ is an unsubstituted C₁-C₆ alkyl, examples of which include, but are not limited to, methyl, ethyl, and n-propyl, preferably methyl. In some embodiments, R₁₀ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one or more of R₉ and R₁₀ is a substituted or unsubstituted C₃-C₁₀ cycloalkyl. In some embodiments, one or more of R₉ and R₁₀ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, one or more of R₉ and R₁₀ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₉ and/or R₁₀ is an unsubstituted or substituted alkenyl, e.g., a unsubstituted or substituted allyl.

In some embodiments, at least one of X₁, X₂, Y₁, Y₂, R₂, R₄, R₅, R₆, R₇, R₉, and R₁₀ comprises deuterium.

In some embodiments, the 5-HT_{2A} receptor agonist is a compound of Formula (II), or a pharmaceutically acceptable salt, stereoisomer, solvate, or prodrug thereof

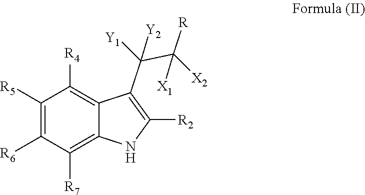

wherein:
X₁ and X₂ are deuterium;
Y₁ and Y₂ are independently selected from the group consisting of hydrogen and deuterium;
R is

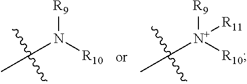

R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkoxy, and substituted or substituted phosphoryloxy;
R₆ and R₇ are independently selected from the group consisting of hydrogen, deuterium, and halogen; and
R₉, R₁₀, and R₁₁ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, one of Y₁ and Y₂ is deuterium while the other is hydrogen.

In some embodiments, R₂ is deuterium. In some embodiments, R₂ is hydrogen. In some embodiments, R₂ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₂ is a substituted C₁-C₆ alkyl. When R₂ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₂ is a substituted or unsubstituted C₃-C₁₀ cycloalkyl. In some embodiments, R₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₂ is an unsubstituted or substituted alkenyl, e.g., a unsubstituted or substituted allyl.

R₄ and R₅ may be the same, or different. In some embodiments, R₄ is deuterium. In some embodiments, R₄ is hydrogen. In some embodiments, R₄ is hydroxy. In some embodiments, R₄ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₄ is a substituted alkoxy. When R₄ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be —OCDH₂, —OCD₂H, —OCD₃, —OCFH₂, —OCF₂H, —OCF₃, etc. In some embodiments, R₄ is an unsubstituted phosphoryloxy group (i.e., —OP(O)(OH)₂ or its deprotonated forms). In some embodiments, R₄ is a substituted phosphoryloxy group where one or more of the hydrogen atoms in —OP(O)(OH)₂ is replaced with a substituent group such as unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, unsubstituted or substituted heteroaryl, or other substituent group as set forth herein. When both hydrogen atoms in —OP(O)(OH)₂ are replaced with a substituent group, the substituent groups can be the same or different from one another.

In some embodiments, R₅ is deuterium. In some embodiments, R₅ is hydrogen. In some embodiments, R₅ is hydroxy. In some embodiments, R₅ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₅ is a substituted alkoxy. When R₅ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be-OCDH₂, —OCD₂H, —OCD₃, —OCFH₂, —OCF₂H, —OCF₃, etc. In some embodiments, R₅ is an unsubstituted phosphoryloxy group (i.e., —OP(O)(OH)₂ or its deprotonated forms). In some embodiments, R₅ is a substituted phosphoryloxy group where one or more of the hydrogen atoms in —OP(O)(OH)₂ is replaced with a substituent group such as unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, unsubstituted or substituted heteroaryl, or other substituent group as set forth herein. When both hydrogen atoms in —OP(O)(OH)₂ are replaced with a substituent group, the substituent groups can be the same or different from one another.

R₆ and R₇ may be the same, or different. R₆ and R₇ may be, independently, hydrogen, deuterium, or a halogen for example —Br, —F, —Cl, or —I.

In some embodiments, R is

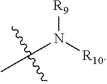

R₉ and R₁₀ may be the same, or different. In some embodiments, R₉ and R₁₀ are the same. In some embodiments, R₉ and R₁₀ are hydrogen. In some embodiments, R₉ and R₁₀ are different. In some embodiments, R₉ is hydrogen, and R₁₀ is a substituted or unsubstituted C₁-C₆ alkyl. In some embodiments, R₁₀ is an unsubstituted C₁-C₆ alkyl, examples of which include, but are not limited to, methyl, ethyl, and n-propyl, preferably methyl. In some embodiments, R₁₀ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one or more of R₉ and R₁₀ is a substituted or unsubstituted C₃-C₁₀ cycloalkyl. In some embodiments, one or more of R₉ and R₁₀ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, one or more of R₉ and R₁₀ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₉ and/or R₁₀ is an unsubstituted or substituted alkenyl, e.g., a unsubstituted or substituted allyl.

In some embodiments, R is an ammonium cation represented by

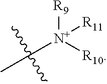

R₉ and R₁₀ are set forth above. R₉, R₁₀, and R₁₁ may be the same, or different. In some embodiments, R₉, R₁₀, and R₁₁ are the same. In some embodiments, R₉, R₁₀, and R₁₁ are each different. In some embodiments, two of R₉, R₁₀, and R₁₁ are the same. In some embodiments, R₁₁ is hydrogen. In some embodiments, Ru is an unsubstituted C₁-C₆ alkyl, examples of which include, but are not limited to, methyl, ethyl, and n-propyl, preferably methyl. In some embodiments, R₁₁ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₁₁ is a substituted or unsubstituted C₃-C₁₀ cycloalkyl. In some embodiments, R₁₁ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₁₁ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₁₁ is an unsubstituted or substituted alkenyl, e.g., a unsubstituted or substituted allyl. In some embodiments, R is a quaternary ammonium cation (where R₉, R₁₀, and R₁₁ are each not hydrogen). In some embodiments, R is a protonated ammonium cation, in which one, two, or three of R₉, R₁₀, and R₁₁ is hydrogen. When R represents either a quaternary ammonium cation or a protonated ammonium cation, R may be accompanied by a suitable conjugate base pair, examples of which include, but are not limited to, the conjugate base of any of acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (+)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, α-oxo-glutaric acid, glycolic acid, hippuric acid, hydrobromic acid, hydrochloric acid, bydroiodic acid, (+)-L-lactic acid, (−)-D-lactic acid, (+)-DL-lactic acid, lactobionic acid, maleic acid, malic acid, (−)-L-malic acid, (+)-D-malic acid, hydroxymaleic acid, malonic acid, (+)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, or a fatty acid (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.).

In some embodiments, the 5-HT_{2A} receptor agonist is a compound of Formula (II-a), or a pharmaceutically acceptable salt, stereoisomer, solvate, or prodrug thereof

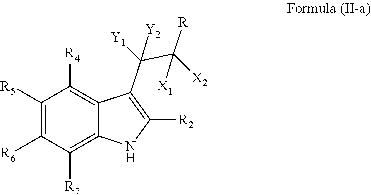

wherein:
X₁ and X₂ are deuterium;
Y₁ and Y₂ are hydrogen;
R is

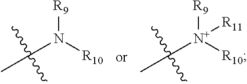

and
R₂, R₄, R₅, R₆, R₇, R₉, R₁₀, and R₁₁ are as defined above for Formula (II).

In some embodiments, the 5-HT_{2A} receptor agonist is a compound of Formula (II-b), or a pharmaceutically acceptable salt, stereoisomer, solvate, or prodrug thereof

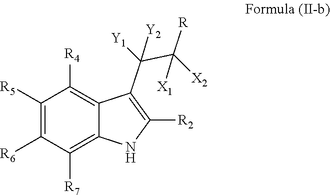

wherein:
X₁ and X₂ are deuterium;
Y₁ and Y₂ are hydrogen;
R is

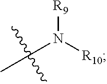

and
R₂, R₄, R₅, R₆, R₇, R₉, and R₁₀ are as defined above for Formula (II).

In some embodiments, the 5-HT_{2A} receptor agonist is a compound of Formula (II-c), or a pharmaceutically acceptable salt, stereoisomer, solvate, or prodrug thereof

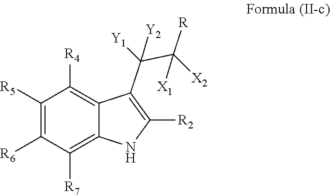

wherein:
X₁ and X₂ are deuterium Y₁ and Y₂ are hydrogen;
R is

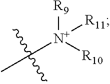

and
R₂, R₄, R₅, R₆, R₇, R₉, R₁₀, and R₁₁ are as defined above for Formula (II).

In some embodiments, the 5-HT_{2A} receptor agonist is a compound of Formula (II-d), or a pharmaceutically acceptable salt, stereoisomer, solvate, or prodrug thereof

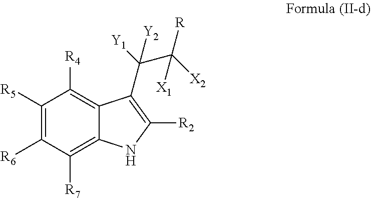

wherein:
X₁ and X₂ are deuterium;
Y₁ and Y₂ are hydrogen;
R is

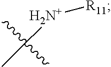

and
R₂, R₅, R₇, and R₁₁ are as defined above for Formula (II).

In preferred embodiments, the 5-HT_{2A} receptor agonist is at least one tryptamine derivative selected from the group consisting of:

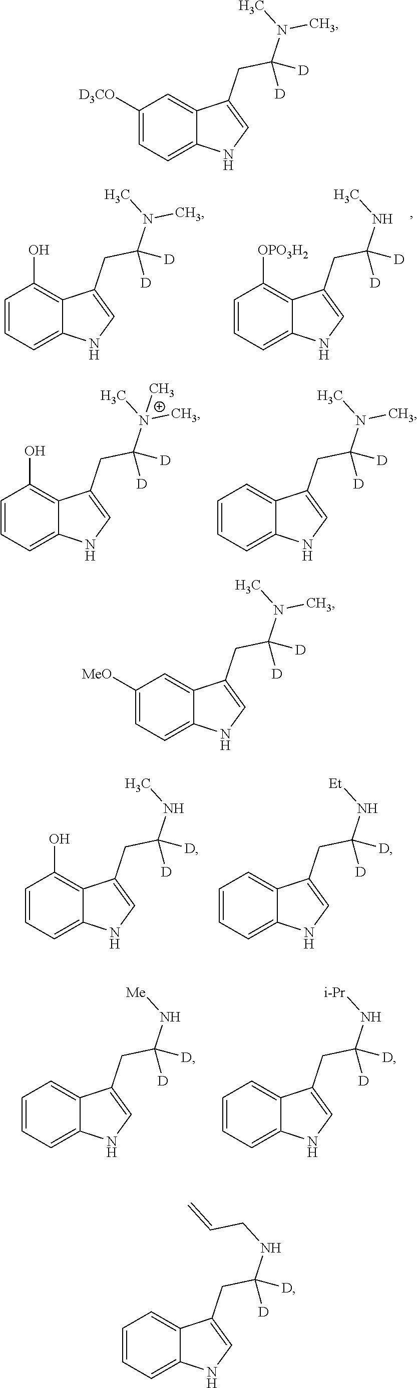

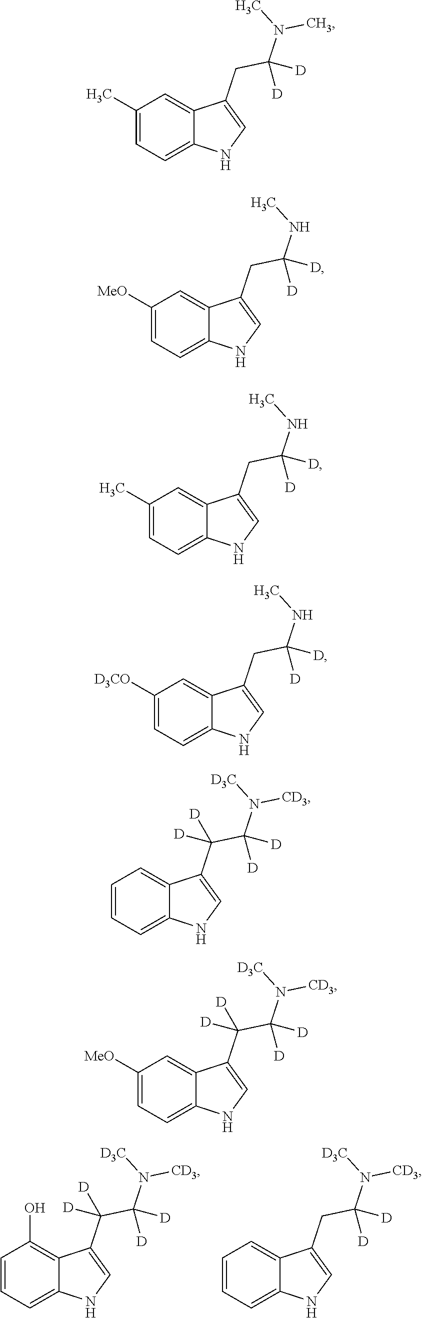

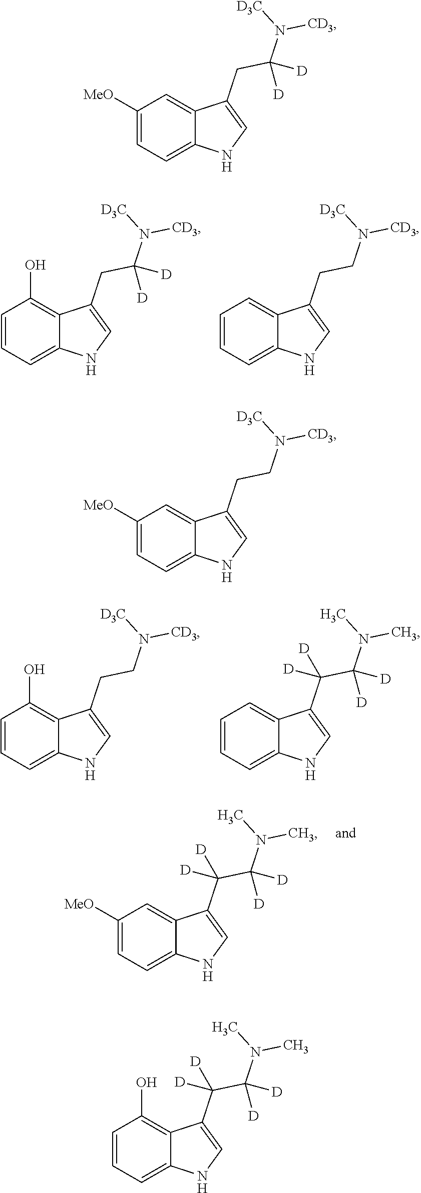

or a pharmaceutically acceptable salt, solvate, or prodrug thereof.

In some embodiments, the 5-HT_{2A} receptor agonist is a compound of Formula (III) or a pharmaceutically acceptable salt, stereoisomer, solvate, or prodrug thereof

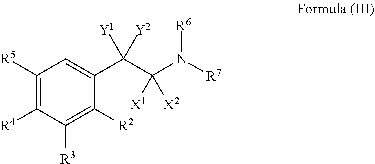

wherein:
X¹ and X² are independently selected from the group consisting of hydrogen, deuterium, and unsubstituted or substituted C₁-C₆ alkyl;
Y¹ and Y² are independently selected from the group consisting of hydrogen and deuterium;
R² and R³ are independently selected from the group consisting of hydrogen, deuterium, halogen, unsubstituted or substituted C₁-C₆ alkyl, and —OR^{a};
R⁴ and R⁵ are independently selected from the group consisting of hydrogen, deuterium, halogen, a substituted or unsubstituted C₁-C₆ alkyl, —OR^{a}, and —SR^{a}, or R⁴ and R⁵ together with the atoms to which they are attached optionally form an unsubstituted or substituted heterocycloalkyl or an unsubstituted or substituted heteroaryl;
R⁶ and R⁷ are independently selected from the group consisting of hydrogen and unsubstituted or substituted C₁-C₆ alkyl; and
each R^{a} is independently selected from the group consisting of hydrogen, deuterium, and unsubstituted or substituted C₁-C₆ alkyl.

X¹ and X² may be the same, or different. In some embodiments, X¹ and X² are the same. In some embodiments, X¹ and X² are hydrogen. In some embodiments, X¹ and X² are deuterium. In some embodiments, X¹ and X² are different. In some embodiments, X¹ is hydrogen or deuterium, and X² is a substituted or unsubstituted C₁-C₆ alkyl. In some embodiments, X² is an unsubstituted C₁-C₆ alkyl, examples of which include, but are not limited to, methyl, ethyl, and n-propyl, preferably methyl. In some embodiments, X² is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X¹ and X² is deuterium while the other is hydrogen.

Y¹ and Y² may be the same, or different. In some embodiments, Y¹ and Y² are the same. In some embodiments, Y¹ and Y² are hydrogen. In some embodiments, Y¹ and Y² are deuterium. In some embodiments, X¹ and X² are different. In some embodiments, one of Y¹ and Y² is deuterium while the other is hydrogen.

In some embodiments, R² is deuterium. In some embodiments, R² is hydrogen. In some embodiments, R² is halogen, for example —Br, —F, —Cl, or —I. In some embodiments, R² is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R² is a substituted C₁-C₆ alkyl. When R² is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, 2 is —OR^{a}.

In some embodiments, R³ is deuterium. In some embodiments, R³ is hydrogen. In some embodiments, R³ is halogen, for example —Br, —F, —Cl, or —I. In some embodiments, R³ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R³ is a substituted C₁-C₆ alkyl. When R³ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, 3 is —OR^{a}.

In some embodiments, R⁴ is deuterium. In some embodiments, R⁴ is hydrogen. In some embodiments, R⁴ is halogen, for example —Br, —F, —Cl, or —I. In some embodiments, 4 is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁴ is a substituted C₁-C₆ alkyl. When R⁴ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁴ is —OR^{a}. In some embodiments, R⁴ is —SR^{a}. In some embodiments, R⁴ is —SMe, —SCD₃, —SCF₃, —SEt, —Sn—Pr, —SCH₂CH₂CF₃, —SCH₂CH₂CF₂H, —SCH₂CH₂CFH₂, —Me, —CD₃, —CF₃, —OMe, —OCD₃, —OCF₃, —OCH₂CH₂CF₃, —OCH₂CH₂CF₂H, —OCH₂CH₂CFH₂, or —Br. In some embodiments, R⁴ is hydrogen, deuterium, halogen, —OR^{a}, or —SR^{a}, and R^{a} is C₁-C₆ alkyl, which is unsubstituted or substituted with one or more deuteriums.

In some embodiments, R⁵ is deuterium. In some embodiments, R⁵ is hydrogen. In some embodiments, R⁵ is halogen, for example —Br, —F, —Cl, or —I. In some embodiments, R⁵ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁵ is a substituted C₁-C₆ alkyl. When R⁵ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁵ is —OR^{a}. In some embodiments, R⁵ is —SR^{a}. In some embodiments, R⁵ is hydrogen, —OMe, or —OCD₃. In some embodiments, R⁵ is hydrogen. In some embodiments, R⁵ is —OMe. In some embodiments, R⁵ is-OCD₃. In some embodiments, R⁵ is hydrogen, deuterium, halogen, —OR^{a}, or —SR^{a}, and R^{a} is C₁-C₆ alkyl, which is unsubstituted or substituted with one or more deuteriums. In some embodiments, R⁴ is —OCH₃, —OCD₃, —Br, —SCH₃, —SCH₂CH₃, or —SCH₂CH₂CH₃, and/or R⁵ is hydrogen, —OMe, or —OCD₃.

In some embodiments, R⁴ and R⁵ together with the atoms attached thereto are joined to form a heterocycloalkyl or heteroaryl, with specific mention being made to a benzo[d][1,3]oxathiole group or a benzo[d][1,3]dioxole group. In embodiments where R⁴ and R⁵ together with the atoms attached thereto are joined to form a heterocycloalkyl or heteroaryl (e.g., benzo[d][1,3]oxathiole group, a benzo[d][1,3]dioxole group, etc.), the heterocycloalkyl or heteroaryl ring (e.g., oxathiole ring, the dioxole ring, etc.) may be further substituted with substituents as defined herein, e.g., with one or more halogen (e.g., fluorine) or deuterium substituents.

R⁶ and R⁷ may be the same, or different. R⁶ and R⁷ may be, independently, hydrogen, an unsubstituted C₁-C₆ alkyl (e.g., methyl, ethyl, n-propyl, isopropyl, n-butyl, isobutyl, sec-butyl, and hexyl) or a C₁-C₆ alkyl substituted with one or more deuterium (e.g., —CDH₂, —CD₂H, —CD₃).

Each R^{a} may be, independently, hydrogen, deuterium, an unsubstituted C₁-C₆ alkyl (e.g., methyl, ethyl, n-propyl, isopropyl, n-butyl, isobutyl, see-butyl, t-butyl, n-pentyl, neopentyl, and hexyl), or a substituted C₁-C₆ alkyl, with preferred substituents including, but not limited to, deuterium, halogen (e.g., fluorine), polar substituents such as hydroxyl or polyether substituents, etc. In some embodiments, R^{a} is a substituted or unsubstituted C₁-C₆ alkyl, preferably a C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is-CD₃. In some embodiments, more than one R^{a} is present. In such cases, each R^{a} may be the same, or different. In some embodiments, each R^{a} is the same. In some embodiments, each R^{a} is different, e.g., one R^{a} is —CH₃, while another is-CD₃. In line with the above, examples of —OR^{a} or —SR^{a} may include, but are not limited to, —SMe, —SCD₃, —SCF₃, —SEt, —Sn—Pr, —SCH₂CH₂CF₃, —SCH₂CH₂CF₂H, —SCH₂CH₂CFH₂, —OMe, —OCD₃, —OCF₃, —OCH₂CH₂CF₃, —OCH₂CH₂CF₂H, and —OCH₂CH₂CFH₂.

In some embodiments, at least one of X¹, X², Y¹, Y², R², R³, R⁴, R⁵, R⁶, and R⁷ comprises deuterium.

In some embodiments, the 5-HT_{2A} receptor agonist is a compound of Formula (III-a) or a pharmaceutically acceptable salt, stereoisomer, solvate, or prodrug thereof

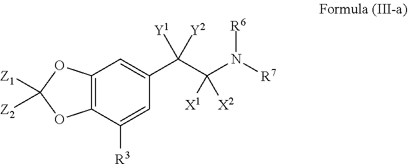

wherein:
Z¹ and Z² are independently selected form the group consisting of hydrogen, deuterium, or fluorine; and
X¹, X², Y¹, Y², R³, R⁶, R⁷, and R^{a} are as defined for Formula (III).

Z¹ and Z² may be the same, or different. In some embodiments, Z¹ and Z² are the same. In some embodiments, Z¹ and Z² are hydrogen. In some embodiments, Z¹ and Z² are deuterium. In some embodiments, Z¹ and Z² are fluorine, some embodiments, Z¹ and Z² are different. In some embodiments, one of Z¹ and Z² is deuterium while the other is hydrogen.

In some embodiments, at least one of Z¹, Z², X¹, X², Y¹, Y², R³, R⁶, and R⁷ comprises deuterium.

In some embodiments, R⁶ and R⁷ are independently hydrogen, —CH₃, or —OCD₃.

In preferred embodiments, the 5-HT_{2A} receptor agonist is at least one phenethylamine derivative selected from the group consisting of:

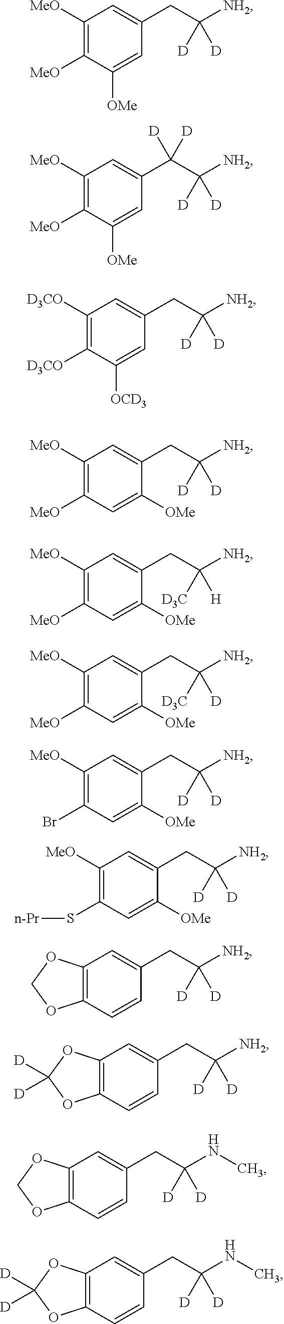

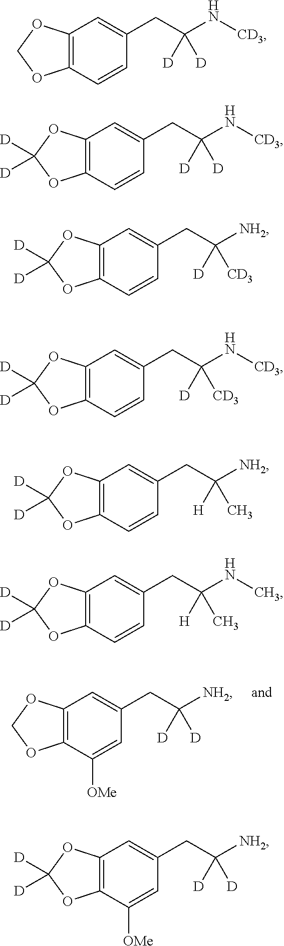

or a pharmaceutically acceptable salt, stereoisomer, solvate, or prodrug thereof.

In some embodiments, the 5-HT_{2A} receptor agonist is an N-substituted phenethylamine (NSP).

In some embodiments, the 5-HT_{2A} receptor agonist is a compound of Formula (IV) or a pharmaceutically acceptable salt, stereoisomer, solvate, or prodrug thereof

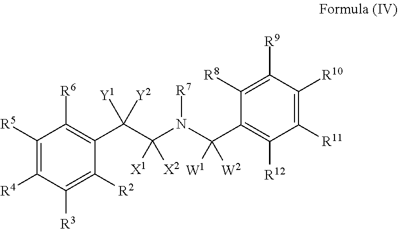

wherein:
R² and R³ are independently selected from the group consisting of hydrogen, deuterium, cyano, halogen, unsubstituted or substituted C₁-C₆ alkyl, —OR^{a}, and —SR^{a}, or R² and R³ together with the atoms to which they are attached optionally form an unsubstituted or substituted cycloalkyl, aryl, heterocycloalkyl, or heteroaryl;
R⁴ is selected from the group consisting of hydrogen, deuterium, cyano, halogen, unsubstituted or substituted C₁-C₆ alkyl, —OR^{a}, and —SR^{a};
R⁵ and R⁶ are independently selected from the group consisting of hydrogen, deuterium, cyano, halogen, unsubstituted or substituted C₁-C₆ alkyl, —OR^{a}, and —SR^{a}, or R⁵ and R⁶ together with the atoms to which they are attached optionally form an unsubstituted or substituted cycloalkyl, aryl, heterocycloalkyl, or heteroaryl;
W¹ and W² are independently selected from the group consisting of hydrogen, deuterium, and unsubstituted or substituted C₁-C₆ alkyl;
X¹ and X² are independently selected from the group consisting of hydrogen, deuterium, and unsubstituted or substituted C₁-C₆ alkyl; or X² and W¹ together with the atoms to which they are attached optionally form an unsubstituted or substituted heterocycloalkyl;
Y¹ and Y² are independently selected from the group consisting of hydrogen, deuterium, and unsubstituted or substituted C₁-C₆ alkyl;
R⁷ is selected from the group consisting of hydrogen, deuterium, and unsubstituted or substituted C₁-C₆ alkyl;
R⁸, R⁹, and R¹⁰ are independently selected from the group consisting of hydrogen, deuterium, hydroxyl, cyano, halogen, unsubstituted or substituted C₁-C₆ alkyl, —OR^{a}, and —SR^{a};
R¹¹ and R¹² are independently selected from the group consisting of hydrogen, deuterium, hydroxyl, cyano, halogen, unsubstituted or substituted C₁-C₆ alkyl, —OR^{a}, and —SR^{a}, or R¹¹ and R¹² together with the atoms to which they are attached optionally form an unsubstituted or substituted cycloalkyl, aryl, heterocycloalkyl, or heteroaryl; and
each R^{a} is independently selected from the group consisting of hydrogen, deuterium, and unsubstituted or substituted C₁-C₆ alkyl.

In some embodiments, R² is deuterium. In some embodiments, R² is hydrogen. In some embodiments, R² is halogen, for example —Br, —F, —Cl, or —I. In some embodiments, R² is cyano. In some embodiments, R² is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R² is a substituted C₁-C₆ alkyl. When R² is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R² is —OR^{a}. In some embodiments, R² is —SR^{a}.

In some embodiments, R³ is deuterium. In some embodiments, R³ is hydrogen. In some embodiments, R³ is halogen, for example —Br, —F, —Cl, or —I. In some embodiments, R³ is cyano. In some embodiments, R³ is a an unsubstituted C₁-C₆ alkyl, examples of which include, but are not limited to, methyl, ethyl, n-propyl, isopropyl, n-butyl, isobutyl, see-butyl, t-butyl, n-pentyl, neopentyl, and hexyl. In some embodiments, R³ is a substituted C₁-C₆ alkyl. When R³ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, 3 is —OR^{a}. In some embodiments, R² is —SR^{a}.

In some embodiments, R² and R³ together with the atoms to which they are attached form an unsubstituted or substituted cycloalkyl, aryl, heterocycloalkyl, or heteroaryl.

In some embodiments, R⁴ is deuterium. In some embodiments, R⁴ is hydrogen. In some embodiments, R⁴ is halogen, for example —Br, —F, —Cl, or —I. In some embodiments, R⁴ is cyano. In some embodiments, R⁴ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁴ is a substituted C₁-C₆ alkyl. When R⁴ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁴ is —OR. In some embodiments, R⁴ is —SR^{a}. In some embodiments, R⁴ is —SMe, —SCD₃, —SCF₃, —SEt, —Sn—Pr, —SCH₂CH₂CF₃, —SCH₂CH₂CF₂H, —SCH₂CH₂CFH₂, —Me, -CD₃, —CF₃, —OMe, —OCD₃, —OCF₃, —OCH₂CH₂CF₃, —OCH₂CH₂CF₂H, —OCH₂CH₂CFH₂, or —Br. In some embodiments, R⁴ is hydrogen, deuterium, halogen, —OR^{a}, or —SR^{a}, and R^{a} is C₁-C₆ alkyl, which is unsubstituted or substituted with one or more deuteriums.

In some embodiments, R⁵ is deuterium. In some embodiments, R⁵ is hydrogen. In some embodiments, R⁵ is halogen, for example —Br, —F, —Cl, or —I. In some embodiments, R⁵ is cyano. In some embodiments, R⁵ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁵ is a substituted C₁-C₆ alkyl. When R⁵ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁵ is —OR^{a}. In some embodiments, R⁵ is —SR^{a}. In some embodiments, R⁵ is hydrogen, —OMe, or —OCD₃. In some embodiments, R⁵ is hydrogen. In some embodiments, R⁵ is —OMe. In some embodiments, R⁵ is-OCD₃. In some embodiments, R⁵ is hydrogen, deuterium, halogen, —OR^{a}, or —SR^{a}, and R^{a} is C₁-C₆ alkyl, which is unsubstituted or substituted with one or more deuteriums.

In some embodiments, R⁶ is deuterium. In some embodiments, R⁶ is hydrogen. In some embodiments, R⁶ is halogen, for example —Br, —F, —Cl, or —I. In some embodiments, R⁶ is cyano. In some embodiments, R⁶ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁶ is a substituted C₁-C₆ alkyl. When R⁶ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁶ is —OR^{a}. In some embodiments, R⁶ is —SR^{a}. In some embodiments, R⁶ is hydrogen, —OMe, or —OCD₃. In some embodiments, R⁶ is hydrogen. In some embodiments, R⁶ is —OMe. In some embodiments, R⁶ is-OCD₃. In some embodiments, R⁶ is hydrogen, deuterium, halogen, —OR^{a}, or —SR, and R^{a} is C₁-C₆ alkyl, which is unsubstituted or substituted with one or more deuteriums.

In some embodiments, R⁵ and R⁶ together with the atoms to which they are attached optionally form an unsubstituted or substituted cycloalkyl, aryl, heterocycloalkyl, or heteroaryl.

W¹ and W² may be the same, or different. In some embodiments, W¹ and W² are the same. In some embodiments, W¹ and W² are hydrogen. In some embodiments, W¹ and W² are deuterium. In some embodiments, W¹ and W² are different. In some embodiments, W¹ is hydrogen or deuterium, and W² is a substituted or unsubstituted C₁-C₆ alkyl. In some embodiments, W² is an unsubstituted C₁-C₆ alkyl, examples of which include, but are not limited to, methyl, ethyl, and n-propyl, preferably methyl. In some embodiments, W² is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of W¹ and W² is deuterium while the other is hydrogen.

X¹ and X² may be the same, or different. In some embodiments, X¹ and X² are the same. In some embodiments, X¹ and X² are hydrogen. In some embodiments, X¹ and X² are deuterium. In some embodiments, X¹ and X² are different. In some embodiments, X¹ is hydrogen or deuterium, and X² is a substituted or unsubstituted C₁-C₆ alkyl. In some embodiments, X² is an unsubstituted C₁-C₆ alkyl, examples of which include, but are not limited to, methyl, ethyl, and n-propyl, preferably methyl. In some embodiments, X² is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X¹ and X² is deuterium while the other is hydrogen.

In some embodiments, X² and W¹ together with the atoms to which they are attached form an unsubstituted or substituted heterocycloalkyl, e.g., a piperidine or pyrrolidine, which may be substituted or unsubstituted.

Y¹ and Y² may be the same, or different. In some embodiments, Y¹ and Y² are the same. In some embodiments, Y¹ and Y² are hydrogen. In some embodiments, Y¹ and Y² are deuterium. In some embodiments, Y¹ and Y² are different. In some embodiments, Y¹ is hydrogen or deuterium, and Y² is a substituted or unsubstituted C₁-C₆ alkyl. In some embodiments, Y² is an unsubstituted C₁-C₆ alkyl, examples of which include, but are not limited to, methyl, ethyl, and n-propyl, preferably methyl. In some embodiments, Y² is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of Y¹ and Y² is deuterium while the other is hydrogen.

In some embodiments R⁷ is hydrogen. In some embodiments R⁷ is deuterium. In some embodiments R⁷ is an unsubstituted C₁-C₆ alkyl (e.g., methyl, ethyl, n-propyl, isopropyl, n-butyl, isobutyl, sec-butyl, and hexyl) or a C₁-C₆ alkyl substituted with one or more substituents, such as one or more deuterium (e.g., —CDH₂, —CD₂H, —CD₃).

R⁸, R⁹, and R¹⁰ may be the same, or different. In some embodiments, R⁸, R⁹, and R¹⁰ are the same. In some embodiments, R⁸, R⁹, and R¹⁰ are each different. In some embodiments, two of R⁸, R⁹, and R¹⁰ are the same.

In some embodiments, R⁸ is deuterium. In some embodiments, R⁸ is hydrogen. In some embodiments, R⁸ is halogen, for example —Br, —F, —Cl, or —I. In some embodiments, R⁸ is hydroxyl. In some embodiments, R⁸ is cyano. In some embodiments, R⁸ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁸ is a substituted C₁-C₆ alkyl. When R⁸ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁸ is —OR. In some embodiments, R⁸ is —SR^{a}. In some embodiments, R⁸ is hydrogen, —OMe, or —OCD₃. In some embodiments, R⁸ is hydrogen. In some embodiments, R⁵ is —OMe. In some embodiments, R⁸ is —OCD₃. In some embodiments, R⁸ is hydrogen, deuterium, halogen, —OR^{a}, or —SR^{a}, and R^{a} is C₁-C₆ alkyl, which is unsubstituted or substituted with one or more deuteriums.

In some embodiments, R⁹ is deuterium. In some embodiments, R⁹ is hydrogen. In some embodiments, R⁹ is halogen, for example —Br, —F, —Cl, or —I. In some embodiments, R⁹ is hydroxyl. In some embodiments, R⁹ is cyano. In some embodiments, 9 is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁹ is a substituted C₁-C₆ alkyl. When 9 is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, ⁹ is —OR. In some embodiments, 9 is —SR^{a}. In some embodiments, R⁹ is hydrogen, —OMe, or —OCD₃. In some embodiments, 9 is hydrogen. In some embodiments, R⁹ is —OMe. In some embodiments, R⁹ is-OCD₃. In some embodiments, R⁹ is hydrogen, deuterium, halogen, —OR^{a}, or —SR^{a}, and R^{a} is C₁-C₆alkyl, which is unsubstituted or substituted with one or more deuteriums.

In some embodiments, R¹⁰ is deuterium. In some embodiments, R¹⁰ is hydrogen. In some embodiments, R¹⁰ is halogen, for example —Br, —F, —Cl, or —I. In some embodiments, R¹⁰ is hydroxyl. In some embodiments, R¹⁰ is cyano. In some embodiments, R¹⁰ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹⁰ is a substituted C₁-C₆ alkyl. When R¹⁰ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R¹ is —OR^{a}. In some embodiments, R¹⁰ is —SR^{a}. In some embodiments, R¹⁰ is hydrogen, —OMe, or —OCD₃. In some embodiments, R¹⁰ is hydrogen. In some embodiments, R¹⁰ is —OMe. In some embodiments, R¹⁰ is-OCD₃. In some embodiments, R¹⁰ is hydrogen, deuterium, halogen, —OR^{a}, or —SR, and R^{a} is C₁-C₆ alkyl, which is unsubstituted or substituted with one or more deuteriums.

R¹¹ and R¹² may be the same or different. In some embodiments, R¹¹ is deuterium. In some embodiments, R¹¹ is hydrogen. In some embodiments, R¹¹ is halogen, for example —Br, —F, —Cl, or —I. In some embodiments, R¹ is hydroxyl. In some embodiments, R¹¹ is cyano. In some embodiments, R¹¹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹¹ is a substituted C₁-C₆ alkyl. When R¹¹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R¹¹ is —OR^{a}. In some embodiments, R¹¹ is —SR^{a}. In some embodiments, R¹ is hydrogen, —OMe, or —OCD₃. In some embodiments, R¹ is hydrogen. In some embodiments, R¹¹ is —OMe. In some embodiments, R¹¹ is-OCD₃. In some embodiments, Ru is hydrogen, deuterium, halogen, —OR^{a}, or —SR^{a}, and R^{a} is C₁-C₆ alkyl, which is unsubstituted or substituted with one or more deuteriums.

In some embodiments, R² is deuterium. In some embodiments, R¹² is hydrogen. In some embodiments, R² is halogen, for example —Br, —F, —Cl, or —I. In some embodiments, R¹² is hydroxyl. In some embodiments, R¹² is cyano. In some embodiments, R¹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R^{t} is a substituted C₁-C₆ alkyl. When R¹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R¹ is —OR^{a}. In some embodiments, R¹ is —SR^{a}. In some embodiments, R¹² is hydrogen, —OMe, or —OCD₃. In some embodiments, R¹² is hydrogen. In some embodiments, R¹² is —OMe. In some embodiments, R¹² is-OCD₃. In some embodiments, R¹² is hydrogen, deuterium, halogen, —OR^{a}, or —SR, and R^{a} is C₁-C₆ alkyl, which is unsubstituted or substituted with one or more deuteriums.

In some embodiments, R¹ and R² together with the atoms to which they are attached form an unsubstituted or substituted cycloalkyl, aryl, heterocycloalkyl, or heteroaryl.

Each R^{a} may be, independently, hydrogen, deuterium, an unsubstituted C₁-C₆ alkyl (e.g., methyl, ethyl, n-propyl, isopropyl, n-butyl, isobutyl, sec-butyl, t-butyl, n-pentyl, neopentyl, and hexyl), or a substituted C₁-C₆ alkyl, with preferred substituents including, but not limited to, deuterium, halogen (e.g., fluorine), polar substituents such as hydroxyl or polyether substituents, etc. In some embodiments, R^{a} is a substituted or unsubstituted C₁-C₆ alkyl, preferably a C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is-CD₃. In some embodiments, more than one R^{a} is present. In such cases, each R^{a} may be the same, or different. In some embodiments, each R^{a} is the same. In some embodiments, each R^{a} is different, e.g., one R^{a} is —CH₃, while another is-CD₃. In line with the above, examples of —OR^{a} or —SR^{a} may include, but are not limited to, —SMe, —SCD₃, —SCF₃, —SEt, —Sn—Pr, —SCH₂CH₂CF₃, —SCH₂CH₂CF₂H, —SCH₂CH₂CFH₂, —OMe, —OCD₃, —OCF₃, —OCH₂CH₂CF₃, —OCH₂CH₂CF₂H, and —OCH₂CH₂CFH₂.

In some embodiments, at least one of W¹, W², X¹, X², Y¹, Y², R², R³, R⁵, R⁶, R⁷, R⁸, R⁹, R¹⁰, R¹¹, R¹² comprises deuterium.

In some embodiments, the 5-HT_{2A} receptor agonist is a compound of Formula (IV-a) or a pharmaceutically acceptable salt, stereoisomer, solvate, or prodrug thereof

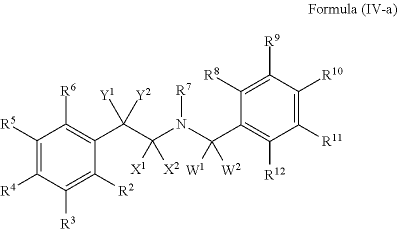

wherein:
X¹ and X² are deuterium; and
W¹, W², Y¹, Y², R², R³, R⁴, R⁵, R⁶, R⁷, R⁸, R⁹, R¹⁰, R¹¹, R¹², and R^{a} are as defined above for Formula (IV).

In some embodiments, at least one of W¹, W², Y¹, Y², R², R³, R⁴, R⁵, R⁶, R⁷, R⁸, R⁹, R¹⁰, R¹¹, and R² comprises deuterium.

In some embodiments, the 5-HT_{2A} receptor agonist is a compound of Formula (IV-b) or a pharmaceutically acceptable salt, stereoisomer, solvate, or prodrug thereof

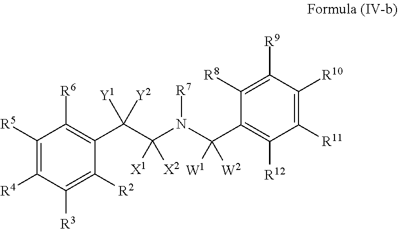

wherein:
W¹ and W² are deuterium; and
X¹, X², Y¹, Y², R², R³, R⁴, R⁵, R⁶, R⁷, R⁸, R⁹, R¹⁰, R¹¹, R¹², and R^{a} are as defined above for Formula (IV).

In some embodiments, at least one of X¹, X², Y¹, Y², R², R³, R⁵, R⁶, R⁷, R⁸, R⁹, R¹⁰, R¹¹, R¹² comprises deuterium.

In some embodiments, the 5-HT_{2A} receptor agonist is a compound of Formula (V) or a pharmaceutically acceptable salt, stereoisomer, solvate, or prodrug thereof

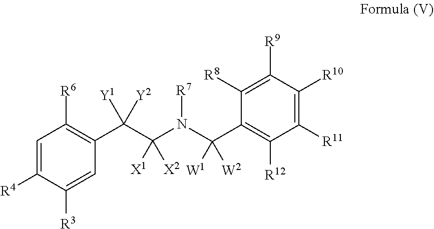

wherein:
R³ and R⁶ are —OR^{a};
R⁴ is selected from the group consisting of hydrogen, deuterium, cyano, halogen, unsubstituted or substituted C₁-C₆ alkyl, —OR^{a}, and -Sa.
W¹ and W² are independently selected from the group consisting of hydrogen, deuterium, and unsubstituted or substituted C₁-C₆ alkyl;
X¹ and X² are independently selected from the group consisting of hydrogen, deuterium, and unsubstituted or substituted C₁-C₆ alkyl;
Y¹ and Y² are independently selected from the group consisting of hydrogen, deuterium, and unsubstituted or substituted C₁-C₆ alkyl;
R⁷ is selected from the group consisting of hydrogen, deuterium, and unsubstituted or substituted C₁-C₆ alkyl;
R⁸, R⁹, and R¹⁰ are independently selected from the group consisting of hydrogen, deuterium, hydroxyl, cyano, halogen, unsubstituted or substituted C₁-C₆ alkyl, —OR^{a}, and —SR^{a};
R¹¹ and R¹² are independently selected from the group consisting of hydrogen, deuterium, hydroxyl, cyano, halogen, unsubstituted or substituted C₁-C₆ alkyl, —OR^{a}, and —SR^{a}, or R¹¹ and R¹² together with the atoms to which they are attached optionally form an unsubstituted or substituted cycloalkyl, aryl, heterocycloalkyl, or heteroaryl; and
each R^{a} is independently selected from the group consisting of hydrogen, deuterium, and unsubstituted or substituted C₁-C₆ alkyl.

In some embodiments, R⁴ is deuterium. In some embodiments, R⁴ is hydrogen. In some embodiments, R⁴ is halogen, for example —Br, —F, —Cl, or —I. In some embodiments, R⁴ is cyano. In some embodiments, R⁴ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁴ is a substituted C₁-C₆ alkyl. When R⁴ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁴ is —OR^{a}. In some embodiments, R⁴ is —SR. In some embodiments, R⁴ is —SMe, —SCD₃, —SCF₃, —SEt, —Sn—Pr, —SCH₂CH₂CF₃, —SCH₂CH₂CF₂H, —SCH₂CH₂CFH₂, —Me, —CD₃, —CF₃, —OMe, —OCD₃, —OCF₃, —OCH₂CH₂CF₃, —OCH₂CH₂CF₂H, —OCH₂CH₂CFH₂, or —Br. In some embodiments, R⁴ is hydrogen, deuterium, halogen, —OR^{a}, or —SR, and R^{a} is C₁-C₆ alkyl, which is unsubstituted or substituted with one or more deuteriums.

W¹ and W² may be the same, or different. In some embodiments, W¹ and W² are the same. In some embodiments, W¹ and W² are hydrogen. In some embodiments, W¹ and W² are deuterium. In some embodiments, W¹ and W² are different. In some embodiments, W¹ is hydrogen or deuterium, and W² is a substituted or unsubstituted C₁-C₆ alkyl. In some embodiments, W² is an unsubstituted C₁-C₆ alkyl, examples of which include, but are not limited to, methyl, ethyl, and n-propyl, preferably methyl. In some embodiments, W² is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of W¹ and W² is deuterium while the other is hydrogen.

X¹ and X² may be the same, or different. In some embodiments, X¹ and X² are the same. In some embodiments, X¹ and X² are hydrogen. In some embodiments, X¹ and X² are deuterium. In some embodiments, X¹ and X² are different. In some embodiments, X¹ is hydrogen or deuterium, and X² is a substituted or unsubstituted C₁-C₆ alkyl. In some embodiments, X² is an unsubstituted C₁-C₆ alkyl, examples of which include, but are not limited to, methyl, ethyl, and n-propyl, preferably methyl. In some embodiments, X² is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X¹ and X² is deuterium while the other is hydrogen.

Y¹ and Y² may be the same, or different. In some embodiments, Y¹ and Y² are the same. In some embodiments, Y¹ and Y² are hydrogen. In some embodiments, Y¹ and Y² are deuterium. In some embodiments, Y¹ and Y² are different. In some embodiments, Y¹ is hydrogen or deuterium, and Y² is a substituted or unsubstituted C₁-C₆ alkyl. In some embodiments, Y² is an unsubstituted C₁-C₆ alkyl, examples of which include, but are not limited to, methyl, ethyl, and n-propyl, preferably methyl. In some embodiments, Y² is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of Y¹ and Y² is deuterium while the other is hydrogen.

In some embodiments R⁷ is hydrogen. In some embodiments R⁷ is deuterium. In some embodiments R⁷ is an unsubstituted C₁-C₆ alkyl (e.g., methyl, ethyl, n-propyl, isopropyl, n-butyl, isobutyl, sec-butyl, and hexyl) or a C₁-C₆ alkyl substituted with one or more substituents, such as one or more deuterium (e.g., —CDH₂, —CD₂H, —CD₃).

R⁸, R⁹, and R¹⁰ may be the same, or different. In some embodiments, R⁸, R⁹, and R¹⁰ are the same. In some embodiments, R⁸, R⁹, and R¹⁰ are each different. In some embodiments, two of R⁸, R⁹, and R¹⁰ are the same.

In some embodiments, R⁸ is deuterium. In some embodiments, R⁸ is hydrogen. In some embodiments, s is halogen, for example —Br, —F, —Cl, or —I. In some embodiments, R⁸ is hydroxyl. In some embodiments, R⁸ is cyano. In some embodiments, R⁸ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, Ba is a substituted C₁-C₆ alkyl. When R⁸ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁸ is —OR^{a}. In some embodiments, R⁸ is —SR^{a}. In some embodiments, R⁸ is hydrogen, —OMe, or —OCD₃. In some embodiments, R⁸ is hydrogen. In some embodiments, R⁸ is —OMe. In some embodiments, BY is-OCD₃. In some embodiments, BY is hydrogen, deuterium, halogen, —OR^{a}, or —SR^{a}, and R^{a} is C₁-C₆ alkyl, which is unsubstituted or substituted with one or more deuteriums.

In some embodiments, R⁹ is deuterium. In some embodiments, R⁹ is hydrogen. In some embodiments, R⁹ is halogen, for example —Br, —F, —Cl, or —I. In some embodiments, R⁹ is hydroxyl. In some embodiments, R⁹ is cyano. In some embodiments, 9 is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁹ is a substituted C₁-C₆ alkyl. When R⁹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁹ is —OR^{a}. In some embodiments, R⁹ is —SR^{a}. In some embodiments, R⁹ is hydrogen, —OMe, or —OCD₃. In some embodiments, R⁹ is hydrogen. In some embodiments, R⁹ is —OMe. In some embodiments, R⁰ is-OCD₃. In some embodiments, R⁹ is hydrogen, deuterium, halogen, —OR^{a}, or —SR^{a}, and R^{a} is C₁-C₆alkyl, which is unsubstituted or substituted with one or more deuteriums.

In some embodiments, R¹⁰ is deuterium. In some embodiments, R¹⁰ is hydrogen. In some embodiments, R¹⁰ is halogen, for example —Br, —F, —Cl, or —I. In some embodiments, R¹⁰ is hydroxyl. In some embodiments, R¹⁰ is cyano. In some embodiments, R¹⁰ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹⁰ is a substituted C₁-C₆ alkyl. When R¹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R¹ is —OR. In some embodiments, R¹⁰ is —SR^{a}. In some embodiments, R¹⁰ is hydrogen, —OMe, or —OCD₃. In some embodiments, R¹⁰ is hydrogen. In some embodiments, R¹⁰ is —OMe. In some embodiments, R¹⁰ is-OCD₃. In some embodiments, R¹⁰ is hydrogen, deuterium, halogen, —OR^{a}, or —SR^{a}, and R^{a} is C₁-C₆ alkyl, which is unsubstituted or substituted with one or more deuteriums.

R¹¹ and R¹² may be the same or different. In some embodiments, R¹¹ is deuterium. In some embodiments, R¹¹ is hydrogen. In some embodiments, R¹¹ is halogen, for example —Br, —F, —Cl, or —I. In some embodiments, R¹¹ is hydroxyl. In some embodiments, R¹¹ is cyano. In some embodiments, R¹¹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹¹ is a substituted C₁-C₆ alkyl. When R¹¹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R¹¹ is —OR^{a}. In some embodiments, R¹ is —SR^{a}. In some embodiments, R¹¹ is hydrogen, —OMe, or —OCD₃. In some embodiments, R¹¹ is hydrogen. In some embodiments, R¹¹ is —OMe. In some embodiments, R¹¹ is-OCD₃. In some embodiments, R¹¹ is hydrogen, deuterium, halogen, —OR^{a}, or —SR^{a}, and R^{a} is C₁-C₆ alkyl, which is unsubstituted or substituted with one or more deuteriums.

In some embodiments, R¹² is deuterium. In some embodiments, R¹² is hydrogen. In some embodiments, R¹² is halogen, for example —Br, —F, —Cl, or —I. In some embodiments, R¹² is hydroxyl. In some embodiments, R¹² is cyano. In some embodiments, R¹² is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹ is a substituted C₁-C₆ alkyl. When R¹² is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R¹² is —OR^{a}. In some embodiments, R¹² is —SR^{a}. In some embodiments, R¹² is hydrogen, —OMe, or —OCD₃. In some embodiments, R¹² is hydrogen. In some embodiments, R¹² is —OMe. In some embodiments, R¹² is-OCD₃. In some embodiments, R¹² is hydrogen, deuterium, halogen, —OR^{a}, or —SR^{a}, and R^{a} is C₁-C₆ alkyl, which is unsubstituted or substituted with one or more deuteriums.

In some embodiments, R¹¹ and R¹² together with the atoms to which they are attached form an unsubstituted or substituted cycloalkyl, aryl, heterocycloalkyl, or heteroaryl.

Each R^{a} may be, independently, hydrogen, deuterium, an unsubstituted C₁-C₆ alkyl (e.g., methyl, ethyl, n-propyl, isopropyl, n-butyl, isobutyl, see-butyl, t-butyl, n-pentyl, neopentyl, and hexyl), or a substituted C₁-C₆ alkyl, with preferred substituents including, but not limited to, deuterium, halogen (e.g., fluorine), polar substituents such as hydroxyl or polyether substituents, etc. In some embodiments, R^{a} is a substituted or unsubstituted C₁-C₆ alkyl, preferably a C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is-CD₃. In some embodiments, more than one R^{a} is present. In such cases, each R^{a} may be the same, or different. In some embodiments, each R^{a} is the same. In some embodiments, each R^{a} is different, e.g., one R^{a} is —CH₃, while another is-CD₃. In line with the above, examples of —OR^{a} or —SR^{a} may include, but are not limited to, —SMe, —SCD₃, —SCF₃, —SEt, —Sn—Pr, —SCH₂CH₂CF₃, —SCH₂H₂CF₂H, —SCH₂CH₂CFH₂, —OMe, —OCD₃, —OCF₃, —OCH₂CH₂CF₃, —OCH₂CH₂CF₂H, and —OCH₂CH₂CFH₂.

In some embodiments, at least one of W¹, W², X₁, X², Y¹, Y², R³, R⁴, R⁶, R⁷, R⁸, R⁹, R¹⁰, R¹¹, and R¹² comprises deuterium.

In some embodiments, the 5-HT_{2A} receptor agonist is a compound of Formula (V-a) or a pharmaceutically acceptable salt, stereoisomer, solvate, or prodrug thereof

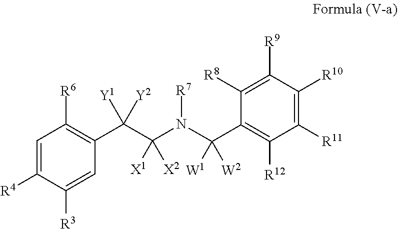

wherein:
R⁸, R⁹, R¹⁰, and R¹¹, are independently selected from the group consisting of hydrogen and deuterium;
R¹² is selected from the group consisting of hydrogen, deuterium, hydroxyl, cyano, halogen, unsubstituted or substituted C₁-C₆ alkyl, —OR^{a}, and —SR^{a}; and
W¹, W², X¹, X², Y¹, Y², R³, R⁴, R², R⁷, and R^{a} are as defined above for Formula (V).

In some embodiments, at least one of W¹, W², X¹, X², Y¹, Y², R³, R⁴, R⁶, R⁷, R⁸, R⁹, R¹⁰, R¹¹, and R¹² comprises deuterium.

In some embodiments, the 5-HT_{2A} receptor agonist is a compound of Formula (V-b) or a pharmaceutically acceptable salt, stereoisomer, solvate, or prodrug thereof

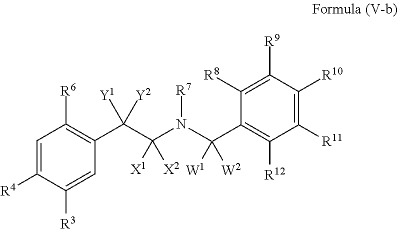

wherein:
R⁸, R⁹, and R¹⁴ are independently selected from the group consisting of hydrogen and deuterium;
R¹¹ and R¹² together with the atoms to which they are attached form an unsubstituted or substituted cycloalkyl, aryl, heterocycloalkyl, or heteroaryl; and
W¹, W², X¹, X², Y¹, Y², R³, R⁴, R⁵, R⁶, and R^{a} are as defined above for Formula (V).

In some embodiments, at least one of W¹, W², X¹, X², Y₁, Y², R³, R⁴, R⁶, R⁷, R⁸, R⁹, R¹⁰, R¹¹, and R¹² comprises deuterium.

In some embodiments, the 5-HT_{2A} receptor agonist is a compound of Formula (VI) or a pharmaceutically acceptable salt, stereoisomer, solvate, or prodrug thereof

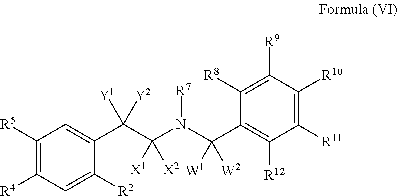

wherein:
R² and R¹ are —OR^{a}
R⁴ is selected from the group consisting of hydrogen, deuterium, cyano, halogen, unsubstituted or substituted C₁-C₆ alkyl, —OR^{a}, and —SR^{a};
W¹ and W² are independently selected from the group consisting of hydrogen, deuterium, and unsubstituted or substituted C₁-C₆ alkyl;
X¹ and X² are independently selected from the group consisting of hydrogen, deuterium, and unsubstituted or substituted C₁-C₆ alkyl;
Y¹ and Y² are independently selected from the group consisting of hydrogen, deuterium, and unsubstituted or substituted C₁-C₆ alkyl;
R⁷ is selected from the group consisting of hydrogen, deuterium, and unsubstituted or substituted C₁-C₆ alkyl;
R⁸, R⁹, and R¹⁰ are independently selected from the group consisting of hydrogen, deuterium, hydroxyl, cyano, halogen, unsubstituted or substituted C₁-C₆ alkyl, —OR^{a}, and —SR^{a};
R¹¹ and R¹² are independently selected from the group consisting of hydrogen, deuterium, hydroxyl, cyano, halogen, unsubstituted or substituted C₁-C₆ alkyl, —OR^{a}, and —SR^{a}, or R¹¹ and R¹² together with the atoms to which they are attached optionally form an unsubstituted or substituted cycloalkyl, aryl, heterocycloalkyl, or heteroaryl; and
each R^{a} is independently selected from the group consisting of hydrogen, deuterium, and unsubstituted or substituted C₁-C₆ alkyl.

In some embodiments, R⁴ is deuterium. In some embodiments, R⁴ is hydrogen. In some embodiments, R⁴ is halogen, for example —Br, —F, —Cl, or —I. In some embodiments, R⁴ is cyano. In some embodiments, R⁴ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁴ is a substituted C₁-C₆ alkyl. When R⁴ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁴ is —OR^{a}. In some embodiments, R⁴ is —SR^{a}. In some embodiments, R⁴ is —SMe, —SCD₃, —SCF₃, —SEt, —Sn—Pr, —SCH₂CH₂CF₃, —SCH₂CH₂CF₂H, —SCH₂CH₂CFH₂, —Me, —CD₃, —CF₃, —OMe, —OCD₃, —OCF₃, —OCH₂CH₂CF₃, —OCH₂CH₂CF₂H, —OCH₂CH₂CFH₂, or —Br. In some embodiments, R⁴ is hydrogen, deuterium, halogen, —OR^{a}, or —SR^{a}, and R^{a} is C₁-C₆ alkyl, which is unsubstituted or substituted with one or more deuteriums.

W¹ and W² may be the same, or different. In some embodiments, W¹ and W² are the same. In some embodiments, W¹ and W² are hydrogen. In some embodiments, W¹ and W² are deuterium. In some embodiments, W¹ and W² are different. In some embodiments, W¹ is hydrogen or deuterium, and W² is a substituted or unsubstituted C₁-C₆ alkyl. In some embodiments, W² is an unsubstituted C₁-C₆ alkyl, examples of which include, but are not limited to, methyl, ethyl, and n-propyl, preferably methyl. In some embodiments, W² is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of W and W² is deuterium while the other is hydrogen.

X¹ and X² may be the same, or different. In some embodiments, X¹ and X² are the same. In some embodiments, X¹ and X² are hydrogen. In some embodiments, X¹ and X² are deuterium. In some embodiments, X¹ and X² are different. In some embodiments, X¹ is hydrogen or deuterium, and X² is a substituted or unsubstituted C₁-C₆ alkyl. In some embodiments, X² is an unsubstituted C₁-C₆ alkyl, examples of which include, but are not limited to, methyl, ethyl, and n-propyl, preferably methyl. In some embodiments, X² is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X¹ and X² is deuterium while the other is hydrogen.

Y¹ and Y² may be the same, or different. In some embodiments, Y¹ and Y² are the same. In some embodiments, Y¹ and Y² are hydrogen. In some embodiments, Y¹ and Y² are deuterium. In some embodiments, Y¹ and Y² are different. In some embodiments, Y¹ is hydrogen or deuterium, and Y² is a substituted or unsubstituted C₁-C₆ alkyl. In some embodiments, Y² is an unsubstituted C₁-C₆ alkyl, examples of which include, but are not limited to, methyl, ethyl, and n-propyl, preferably methyl. In some embodiments, Y₂ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of Y¹ and Y² is deuterium while the other is hydrogen.

In some embodiments R⁷ is hydrogen. In some embodiments R⁷ is deuterium. In some embodiments R⁷ is an unsubstituted C₁-C₆ alkyl (e.g., methyl, ethyl, n-propyl, isopropyl, n-butyl, isobutyl, sec-butyl, and hexyl) or a C₁-C₆ alkyl substituted with one or more substituents, such as one or more deuterium (e.g., —CDH₂, —CD₂H, —CD₃).

R⁸, R⁹, and R¹⁰ may be the same, or different. In some embodiments, R⁸, R⁹, and R¹⁰ are the same. In some embodiments, R⁸, R⁹, and R¹⁰ are each different. In some embodiments, two of R⁸, R⁹, and R¹⁰ are the same.

In some embodiments, R⁸ is deuterium. In some embodiments, R⁸ is hydrogen. In some embodiments, R⁸ is halogen, for example —Br, —F, —Cl, or —I. In some embodiments, R⁸ is hydroxyl. In some embodiments, R⁸ is cyano. In some embodiments, R⁸ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁸ is a substituted C₁-C₆ alkyl. When R⁸ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁸ is —OR^{a}. In some embodiments, R⁸ is —SR^{a}. In some embodiments, R⁸ is hydrogen, —OMe, or —OCD₃. In some embodiments, R⁸ is hydrogen. In some embodiments, R⁸ is —OMe. In some embodiments, R⁸ is-OCD₃. In some embodiments, R⁸ is hydrogen, deuterium, halogen, —OR^{a}, or —SR^{a}, and R^{a} is C₁-C₆ alkyl, which is unsubstituted or substituted with one or more deuteriums.

In some embodiments, R⁹ is deuterium. In some embodiments, R⁹ is hydrogen. In some embodiments, R⁹ is halogen, for example —Br, —F, —Cl, or —I. In some embodiments, R⁹ is hydroxyl. In some embodiments, R⁹ is cyano. In some embodiments, R⁹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁹ is a substituted C₁-C₆ alkyl. When R⁹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁹ is —OR^{a}. In some embodiments, R⁹ is —SR^{a}. In some embodiments, R⁹ is hydrogen, —OMe, or —OCD₃. In some embodiments, R⁹ is hydrogen. In some embodiments, R⁹ is —OMe. In some embodiments, R⁹ is-OCD₃. In some embodiments, R⁹ is hydrogen, deuterium, halogen, —OR^{a}, or —SR^{a} and R^{a} is C₁-C₆ alkyl, which is unsubstituted or substituted with one or more deuteriums.

In some embodiments, R¹⁰ is deuterium. In some embodiments, R¹⁰ is hydrogen. In some embodiments, R¹⁰ is halogen, for example —Br, —F, —Cl, or —I. In some embodiments, R⁰ is hydroxyl. In some embodiments, R¹⁰ is cyano. In some embodiments, R¹⁰ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁷⁰ is a substituted C₁-C₆ alkyl. When R¹⁰ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R¹⁰ is —OR^{a}. In some embodiments, R¹⁰ is —SR^{a}. In some embodiments, R¹⁰ is hydrogen, —OMe, or —OCD₃. In some embodiments, R¹⁰ is hydrogen. In some embodiments, R¹⁰ is —OMe. In some embodiments, R¹⁰ is-OCD₃. In some embodiments, R¹⁰ is hydrogen, deuterium, halogen, —OR^{a}, or —SR, and R^{a} is C₁-C₆ alkyl, which is unsubstituted or substituted with one or more deuteriums.

R¹¹ and R¹² may be the same or different. In some embodiments, R¹¹ is deuterium. In some embodiments, R¹¹ is hydrogen. In some embodiments, R¹¹ is halogen, for example —Br, —F, —Cl, or —I. In some embodiments, R¹¹ is hydroxyl. In some embodiments, R¹¹ is cyano. In some embodiments, R¹¹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹¹ is a substituted C₁-C₆ alkyl. When R¹¹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e. methyl group), the substituted C₁ alkyl group may be —CDH₂, —CD₂H, —CD₃, —CFH₂, —CF₂H, —CF₃, etc. In some embodiments, R¹¹ is —OR^{a}. In some embodiments, R¹¹ is —SR^{a}. In some embodiments, R¹¹ is hydrogen, —OMe, or —OCD₃. In some embodiments, R¹¹ is hydrogen. In some embodiments, R¹¹ is —OMe. In some embodiments, R¹¹ is-OCD₃. In some embodiments, R¹¹ is hydrogen, deuterium, halogen, —OR^{a}, or —SR^{a}, and R^{a} is C₁-C₆ alkyl, which is unsubstituted or substituted with one or more deuteriums.

In some embodiments, R¹² is deuterium. In some embodiments, R¹² is hydrogen. In some embodiments, R¹² is halogen, for example —Br, —F, —Cl, or —I. In some embodiments, R¹² is hydroxyl. In some embodiments, R¹² is cyano. In some embodiments, R¹² is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹² is a substituted C₁-C₆ alkyl. When R¹² is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R¹² is —OR. In some embodiments, R¹² is —SR. In some embodiments, R¹² is hydrogen, —OMe, or —OCD₃. In some embodiments, R¹² is hydrogen. In some embodiments, R¹² is —OMe. In some embodiments, R¹² is-OCD₃. In some embodiments, R¹² is hydrogen, deuterium, halogen, —OR^{a}, or —SR^{a}, and R^{a} is C₁-C₆ alkyl, which is unsubstituted or substituted with one or more deuteriums.

In some embodiments, R¹¹ and R¹² together with the atoms to which they are attached form an unsubstituted or substituted cycloalkyl, aryl, heterocycloalkyl, or heteroaryl.

Each R^{a} may be, independently, hydrogen, deuterium, an unsubstituted C₁-C₆ alkyl (e.g., methyl, ethyl, n-propyl, isopropyl, n-butyl, isobutyl, sec-butyl, t-butyl, n-pentyl, neopentyl, and hexyl), or a substituted C₁-C₆ alkyl, with preferred substituents including, but not limited to, deuterium, halogen (e.g., fluorine), polar substituents such as hydroxyl or polyether substituents, etc. In some embodiments, R^{a} is a substituted or unsubstituted C₁-C₆ alkyl, preferably a C₁-C₃ alkyl, preferably a substituted or substituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is-CD₃. In some embodiments, more than one R^{a} is present. In such cases, each R^{a} may be the same, or different. In some embodiments, each R^{a} is the same. In some embodiments, each R^{a} is different, e.g., one R¹¹ is —CH₃, while another is-CD₃. In line with the above, examples of —OR^{a} or —SR^{a} may include, but are not limited to, —SMe, —SCD₃, —SCF₃, —SEt, —Sn—Pr, —SCH₂CH₂CF₃, —SCH₂CH₂CF₂H, —SCH₂CH₂CFH₂, —OMe, —OCD₃, —OCF₃, —OCH₂CH₂CF₃, —OCH₂CH₂CF₂H, and —OCH₂CH₂CFH₂.

In some embodiments, at least one of W¹, W², X¹, X², Y¹, Y², R², R⁴, R⁵, R⁷, R⁸, R⁹, R¹⁰, R¹¹, and R¹² comprises deuterium.

In some embodiments, the 5-HT_{2A} receptor agonist is a compound of Formula (VI-a) or a pharmaceutically acceptable salt, stereoisomer, solvate, or prodrug thereof

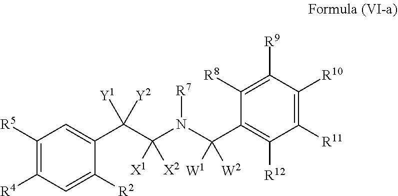

wherein:
R⁸, R⁹, R¹⁰, and R¹¹ are independently selected from the group consisting of hydrogen and deuterium;
R¹² is selected from the group consisting of hydrogen, deuterium, hydroxyl, cyano, halogen, unsubstituted or substituted C₁-C₆ alkyl, —OR^{a}, and —SR^{a}; and
W¹, W², X¹, X², Y₁, Y², R², R⁴, R⁵, R⁷, and R^{a} are as defined above for Formula (VI).

In some embodiments, at least one of W¹, W², X¹, X², Y¹, Y², R², R⁴, R⁵, R⁷, R⁸, R⁹, R₁₀, R¹¹, and R¹² comprises deuterium.

In some embodiments, the 5-HT_{2A} receptor agonist is a compound of Formula (VI-b) or a pharmaceutically acceptable salt, stereoisomer, solvate, or prodrug thereof

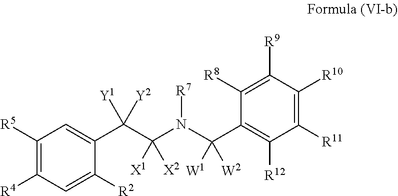

wherein:
R⁸, R⁹, and R¹⁰ are independently selected from the group consisting of hydrogen and deuterium;
R¹¹ and R¹² together with the atoms to which they are attached form an unsubstituted or substituted cycloalkyl, aryl, heterocycloalkyl, or heteroaryl; and
W¹, W², X¹, X², Y¹, Y², R², R⁴, R⁵, R⁷, and R^{a} are as defined above for Formula (VI).

In some embodiments, at least one of W, W², X¹, X², Y¹, Y², R², R⁴, R⁵, R⁷, R⁸, R⁹, R¹⁰, R¹¹, and R¹² comprises deuterium.

In preferred embodiments, the 5-HT_{2A} receptor agonist is at least one N-substituted phenethylamine (NSP) having at least one deuterium atom, which is at least one selected from the group consisting of:

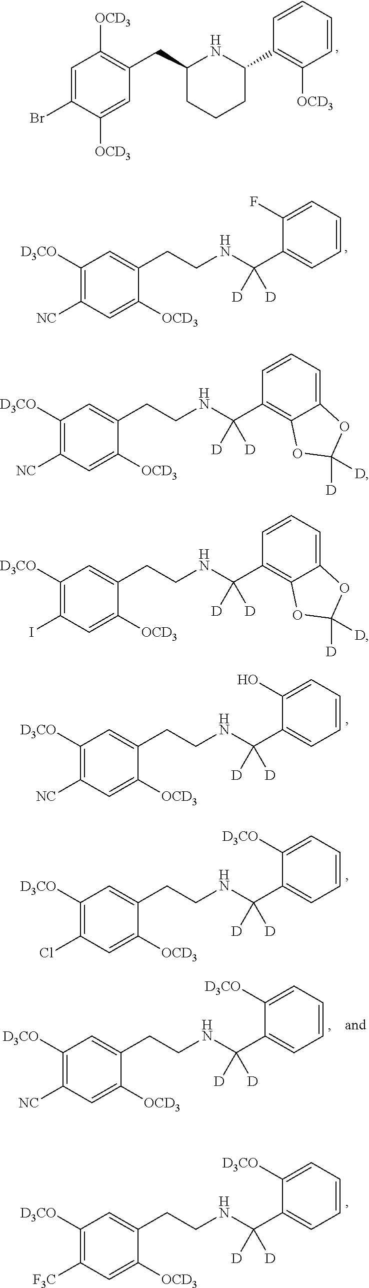

or a pharmaceutically acceptable salt, stereoisomer, solvate, or prodrug thereof.

Also disclosed herein is a pharmaceutically acceptable salt form of the compounds disclosed herein as the 5-HT_{2A} receptor agonist. The acid used to form the pharmaceutically acceptable salt form may be a monoacid, a diacid, a triacid, a tetraacid, or may contain a higher number of acid groups. The acid groups may be, e.g., a carboxylic acid, a sulfonic acid, a phosphonic acid, or other acidic moieties containing at least one replaceable hydrogen atom. Examples of acids for use in the preparation of the pharmaceutically acceptable (acid addition) salts disclosed herein include, but are not limited to, acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (+)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (−)-D-lactic acid, (+)-DL-lactic acid, lactobionic acid, maleic acid, malic acid, (−)-L-malic acid, (+)-D-malic acid, hydroxymaleic acid, malonic acid, (+)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, and fatty acids (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.).

In some embodiments, the salt is formed with N,N-dimethyltryptamine (DMT), 5-hydroxy-N,N-dimethyltryptamine (5-OH-DMT), 5-methoxy-N,N-dimethyltryptamine (5-MeO-DMT), DMT-d₁₀(2-(1H-indol-3-yl)-N,N-bis(methyl-d₃)ethan-1-amine-1,1,2,2-d₄) or 5-MeO-DMT-d₁₀(2-(5-methoxy-1H-indol-3-yl)-N,N-bis(methyl-d₃)ethan-1-amine-1,1,2,2-d₄).

In some embodiments, the pharmaceutically acceptable salt is a fumarate, a benzoate, a salicylate, a succinate, an oxalate, a glycolate, a hemi-oxalate, or a hemi-fumarate salt. In terms of providing desirable physical and pharmaceutical characteristics, such as those described above, preferred pharmaceutically acceptable salts are fumarate salts, benzoate salts, salicylates, and succinate salts of the compounds disclosed herein, e.g., the 5-HT_{2A} receptor agonist, with fumarate, benzoate, and salicylate salts being particularly preferred.

In some embodiments, the pharmaceutically acceptable salt is a fumarate, a benzoate, a salicylate, a succinate, an oxalate, a glycolate, a hemi-oxalate, or a hemi-fumarate salt of N,N-dimethyltryptamine (DMT). In some embodiments, the pharmaceutically acceptable salt is a fumarate, a benzoate, a salicylate, a succinate, an oxalate, a glycolate, a hemi-oxalate, or a hemi-fumarate salt of 5-hydroxy-N,N-dimethyltryptamine (5-OH-DMT). In some embodiments, the pharmaceutically acceptable salt is a fumarate, a benzoate, a salicylate, a succinate, an oxalate, a glycolate, a hemi-oxalate, or a hemi-fumarate salt of 5-methoxy-N,N-dimethyltryptamine (5-MeO-DMT). In some embodiments, the pharmaceutically acceptable salt is a fumarate, a benzoate, a salicylate, a succinate, an oxalate, a glycolate, a hemi-oxalate, or a hemi-fumarate salt of 2-(1H-indol-3-yl)-N,N-bis(methyl-d₃)ethan-1-amine-1,1,2,2-d₄ (DMT-d₁₀). In some embodiments, the pharmaceutically acceptable salt is a fumarate, a benzoate, a salicylate, a succinate, an oxalate, a glycolate, a hemi-oxalate, or a hemi-fumarate salt of 5-MeO-DMT-d₁₀ (2-(5-methoxy-1H-indol-3-yl)-N,N-bis(methyl-d₃)ethan-1-amine-1,1,2,2-d₄).

In some embodiments, the pharmaceutically acceptable salt is a fumarate salt of 2-(1H-indol-3-yl)-N,N-dimethylethan-1-amine (DMT). In some embodiments, the salt is in a crystalline solid form characterized by an X-ray powder diffraction pattern containing at least three characteristic peaks at diffraction angles (2θ±0.2°) selected from the group consisting of 7.8°, 10.3°, 10.9°, 13.6°, 15.8°, 16.1°, 17.0°, 18.4°, 19.7°, 19.9°, 20.6°, 21.3°, 21.7°, 22.5°, 23.9°, 24.1°, 25.1°, 26.2°, 33.6°, and 34.9°, as determined by XRPD using a CuKα radiation source.

In some embodiments, the pharmaceutically acceptable salt is a benzoate salt of 2-(1H-indol-3-yl)-N,N-dimethylethan-1-amine (DMT). In some embodiments, the salt is in a crystalline solid form characterized by an X-ray powder diffraction pattern containing at least three characteristic peaks at diffraction angles (2θ±0.2°) selected from the group consisting of 9.6°, 11.1°, 12.6°, 13.5°, 15.8°, 16.1°, 17.1°, 17.9°, 19.8°, 20.1°, 20.8°, 21.2°, 22.7°, 23.8°, 24.6°, 26.9°, 29.2°, 32.3°, 35.1°, and 36.1°, as determined by XRPD using a CuKα radiation source.

In some embodiments, the pharmaceutically acceptable salt is a salicylate salt of 2-(1H-indol-3-yl)-N,N-dimethylethan-1-amine (DMT). In some embodiments, the salt is in a crystalline solid form characterized by an X-ray powder diffraction pattern containing at least three characteristic peaks at diffraction angles (2θ±0.2°) selected from the group consisting of 9.6°, 10.5°, 14.9°, 17.1°, 18.1°, 19.1°, 20.1°, 20.7°, 21.0°, 21.3°, 24.6°, 25.6°, 28.5°, 28.8°, 29.4°, 30.3°, 31.3°, 32.1°, 33.5°, and 34.40, as determined by XRPD using a CuKα radiation source.

In some embodiments, the pharmaceutically acceptable salt is a fumarate salt of 2-(1H-indol-3-yl)-N,N-bis(methyl-d₃)ethan-1-amine-1,1,2,2-d₄ (DMT-d₁₀). In some embodiments, the salt is in a crystalline solid form characterized by an X-ray powder diffraction pattern containing at least three characteristic peaks at diffraction angles (2θ±0.2°) selected from the group consisting of 7.8°, 10.3°, 10.9°, 13.6°, 15.8°, 16.1°, 17.0°, 18.4°, 19.7°, 19.9°, 20.6°, 21.3°, 21.7°, 22.5°, 23.9°, 24.1°, 25.1°, 26.2°, 33.6°, and 34.9°, as determined by XRPD using a CuKα radiation source.

In some embodiments, the pharmaceutically acceptable salt is a benzoate salt of 2-(1H-indol-3-yl)-N,N-bis(methyl-d₃)ethan-1-amine-1,1,2,2-d₄ (DMT-d₁₀). In some embodiments, the salt is in a crystalline solid form characterized by an X-ray powder diffraction pattern containing at least three characteristic peaks at diffraction angles (2θ±0.2°) selected from the group consisting of 9.6°, 11.1°, 12.6°, 13.5°, 15.8°, 16.1°, 17.1°, 17.9°, 19.8°, 20.1°, 20.8°, 21.2°, 22.7°, 23.8°, 24.6°, 26.9°, 29.2°, 32.3°, 35.1°, and 36.1°, as determined by XRPD using a CuKα radiation source.

In some embodiments, the pharmaceutically acceptable salt is a salicylate salt of 2-(1H-indol-3-yl)-N,N-bis(methyl-d₃)ethan-1-amine-1,1,2,2-d₄ (DMT-d₁₀). In some embodiments, the salt is in a crystalline solid form characterized by an X-ray powder diffraction pattern containing at least three characteristic peaks at diffraction angles (2θ±0.2°) selected from the group consisting of 9.6°, 10.5°, 14.9°, 17.1°, 18.1°, 19.1°, 20.1°, 20.7°, 21.0°, 21.3°, 24.6°, 25.6°, 28.5°, 2.8°, 29.4°, 30.3°, 31.3°, 32.1°, 33.5° and 34.4°, as determined by XRPD using a CuKα radiation source.

In some embodiments, the 5-HT_{2A} receptor agonist of the present disclosure, or any pharmaceutically acceptable salts, stereoisomers, or prodrugs thereof, is in the form of a solvate. Examples of solvate forms include, but are not limited to, hydrates, methanolates, ethanolates, isopropanolates, etc., with hydrates and ethanolates being preferred. The solvate may be formed from stoichiometric or nonstoichiometric quantities of solvent molecules. In one non-limiting example, as a hydrate, the 5-HT_{2A} receptor agonist may be a monohydrate, a dihydrate, etc. Solvates of the compounds herein also include solution-phase forms. Thus, in some embodiments, the present disclosure provides solution-phase compositions of the 5-HT_{2A} receptor agonist of the present disclosure, or any pharmaceutically acceptable salts, stereoisomers, or prodrugs thereof, which are in solvated form, preferably fully solvated form. For example, pharmaceutically acceptable salt forms of the 5-HT_{2A} receptor agonist can be prepared in solution-phase, whereby the salt is pre-formed as a solid and then dissolved in solvent (e.g., water). Alternatively, pharmaceutically acceptable salt forms of the 5-HT_{2A} receptor agonist can be prepared in solution-phase, by mixing the 5-HT_{2A} receptor agonist (free base) with an appropriate acid in solvent (e.g., water) thereby forming the solvated salt form in-situ. If desired, these preparations can be stored as a solution, such as in the form of an aqueous solution, an organic solvent solution, or a mixed aqueous-organic solvent solution, for prolonged periods of time without appreciable degradation or physical changes, such as oiling out of solution. Solvents which can be used to form the solution-phase compositions can be any one or more solvents set forth herein, e.g., water, ethanol, etc. In some embodiments, the solution-phase composition is an aqueous solution-phase composition comprising the 5-HT_{2A} receptor agonist, or a pharmaceutically acceptable salt, stereoisomer, or prodrug thereof, solvated with water.

The 5-HT_{2A} receptor agonist may contain a stereogenic center. In such cases, the compounds may exist as different stereoisomeric forms, even though the chemical Formulae/name are drawn/written without reference to stereochemistry. Accordingly, the present disclosure includes all possible stereoisomers and includes not only racemic compounds but the individual enantiorers (enantiomerically pure compounds), individual diastereomers (diastereomerically pure compounds), and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

In some embodiments, the compounds described herein, e.g., the 5-HT_{2A} receptor agonist, is non-stereogenic. In some embodiments, the compounds described herein, e.g., the 5-HT_{2A} receptor agonist, is racemic. In some embodiments, the compounds described herein, e.g., the 5-HT_{2A} receptor agonist, is enantiomerically enriched (one enantiomer is present in a higher percentage), including enantiomerically pure. In some embodiments, the compounds described herein, e.g., the 5-HT_{2A} receptor agonist, is provided as a single diastereomer. In some embodiments, the compounds described herein, e.g., 5-HT_{2A} receptor agonist, is provided as a mixture of diastereomers. When provided as a mixture of diastereomers, the mixtures may include equal mixtures, or mixtures which are enriched with a particular diastereomer (one diastereomer is present in a higher percentage than another).

### NMDA Receptor Antagonists

As used herein, a “NMDA receptor antagonist” refers to a compound that decreases or inhibits the action of an N-methyl-D-aspartate (NMDA) receptor. Non-limiting examples of NMDA receptor antagonists suitable for use in the present disclosure include, but are not limited to, ketamine, nitrous oxide, memantine, amantadine, dextromethorphan (DXM), phencyclidine (PCP), methoxetamine (MXE), dizocilpine (MK-801), or a combination thereof, including pharmaceutically acceptable salts, stereoisomers, solvates, or prodrugs thereof. In some embodiments, the NMDA receptor antagonist of the combined drug therapy is at least one selected from the group consisting of ketamine, nitrous oxide, memantine, and dextromethorphan, or a pharmaceutically acceptable salt, stereoisomer, solvate, or prodrug thereof.

In some embodiments, the NMDA receptor antagonist is ketamine or a pharmaceutically acceptable salt, stereoisomer, solvate, or prodrug thereof (e.g., (S)-ketamine).

Pharmaceutically acceptable salts of the NMDA receptor antagonist are contemplated herein. The acid used to form the pharmaceutically acceptable salt are those set forth herein.

In some embodiments, the NMDA receptor antagonist of the present disclosure, or any pharmaceutically acceptable salts, stereoisomers, or prodrugs thereof, is in the form of a solvate. Examples of solvate forms include, but are not limited to, hydrates, methanolates, ethanolates, isopropanolates, etc., with hydrates and ethanolates being preferred. The solvate may be formed from stoichiometric or nonstoichiometric quantities of solvent molecules. In one non-limiting example, as a hydrate, the NMDA receptor antagonist may be a monohydrate, a dihydrate, etc. Solvates of the compounds herein also include solution-phase forms. Thus, in some embodiments, the present disclosure provides solution-phase compositions of the NMDA receptor antagonist of the present disclosure, or any pharmaceutically acceptable salts, stereoisomers, or prodrugs thereof, which are in solvated form, preferably fully solvated form. For example, the NMDA receptor antagonist can be prepared in solution-phase through dissolution in solvent (e.g., water). Solvents which can be used to form the solution-phase compositions can be any one or more solvents set forth herein, e.g., water, ethanol, etc. In some embodiments, the solution-phase composition is an aqueous solution-phase composition comprising the NMDA receptor antagonist or any salt, stereoisomer, or prodrug thereof, solvated with water.

The NMDA receptor antagonist may contain a stereogenic center, as is the case with ketamine, for example. In such cases, the compounds may exist as different stereoisomeric forms, even though the chemical Formulae/name are drawn/written without reference to stereochemistry. Accordingly, the present disclosure includes all possible stereoisomers and includes not only racemic compounds but the individual enantiomers (enantiomerically pure compounds), individual diastereomers (diastereomerically pure compounds), and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

In some embodiments, the NMDA receptor antagonist is non-stereogenic. In some embodiments, the NMDA receptor antagonist is racemic. In some embodiments, the NMDA receptor antagonist is enantiomerically enriched (one enantiomer is present in a higher percentage), including enantiomerically pure. In some embodiments, the NMDA receptor antagonist is provided as a single diastereomer. In some embodiments, NMDA receptor antagonist is provided as a mixture of diastereomers. When provided as a mixture of diastereomers, the mixtures may include equal mixtures, or mixtures which are enriched with a particular diastereomer (one diastereomer is present in a higher percentage than another).

In some embodiments, the NMDA receptor antagonist is nitrous oxide and/or memantine, preferably nitrous oxide. In preferred embodiments, the NMDA receptor antagonist is nitrous oxide.

Nitrous oxide, commonly known as laughing gas, is an NMDA receptor antagonist used in a number of medical and dental applications, mostly for pain reduction during surgical procedures. Nitrous oxide is used as a rapid and effective analgesic gas that has a fast onset. Nitrous oxide is also a dissociative inhalant known to cause increased feelings of euphoria, a heightened pain threshold, and involuntary laughing. Furthermore, unlike ketamine, nitrous oxide is not addictive. For these reasons, the use of nitrous oxide as the NMDA receptor antagonist is preferred.

In some embodiments, the combination drug therapy involves providing the 5-HT_{2A} receptor agonist and the NMDA receptor antagonist as a single dosage form for administration to a patient (e.g., each is combined to provide a single aerosol that is inhaled by the patient; or each is combined into a single transdermal patch and delivered transdermally or subcutaneously to the patient). For example, when the NMDA receptor antagonist is nitrous oxide, the 5-HT_{2A} receptor agonist may be present in the liquid phase of the aerosol, while the nitrous oxide may be present in the gas phase of the aerosol. The nitrous oxide (or therapeutic gas mixture comprising nitrous oxide) may be used in the generation of the aerosol or as a carrier gas used to deliver a generated aerosol to the patient. When a generated aerosol is combined with a carrier gas, the carrier gas becomes a part of the gas phase of the aerosol, i.e., the liquid phase of the aerosol becomes entrained in/diluted by the carrier gas. In some embodiments, the combination drug therapy involves providing the 5-HT_{2A} receptor agonist and the NMDA receptor antagonist as separate dosage forms. For example, the 5-HT_{2A} receptor agonist may be provided as an aerosol, preferably a mist, while the NMDA receptor antagonist is provided separately as a therapeutic gas mixture. Alternatively, the 5-HT_{2A} receptor agonist may be provided as an injectable (e.g., intravenous), bolus, infusion, perfusion, etc., while the NMDA receptor antagonist is provided as a therapeutic gas mixture for inhalation delivery.

The co-action of the 5-HT_{2A} receptor agonist and a NMDA receptor antagonist (e.g., nitrous oxide, ketamine, etc.) may provide multiple benefits. For example, the NMDA receptor antagonist may control and/or reduce the activating effects of the 5-HT₂Rs, thereby reducing the risk of overstimulation and occurrences of psychiatric adverse effects such as acute psychedelic crisis. Additionally, administration of the NMDA receptor antagonist may enable the use of a reduced therapeutic dose of the 5-HT_{2A} receptor agonist, thereby decreasing the likelihood of a negative patient experience or dose-dependent side effects. Similarly, administration of the 5-HT_{2A} receptor agonist may reduce the amount of NMDA receptor antagonist necessary for a therapeutic effect, which in the case of NMDA receptor antagonists such as nitrous oxide may alleviate certain side effects such as induced involuntary laughter and the general feelings of anxiety associated therewith. Thus, it is believed that co-administration would reduce the likelihood of a negative experience from the psychedelic administration, either because less psychedelic would be administered or the NMDA receptor antagonist (e.g., nitrous oxide, ketamine, etc.) would enable more efficient functioning of the psychedelic. Similarly, such co-administration would reduce the time or amount of NMDA receptor antagonist (e.g., nitrous oxide, ketamine, etc.) necessary for a therapeutic effect.

NMDA receptor antagonists (e.g., nitrous oxide) and 5-HT_{2A} receptor agonists function via different pharmacological pathways. However, both pathways appear to ultimately converge in a cascade at mTOR (mammalian target of rapamycin, or mechanistic target of rapamycin). Thus, a shared mechanism of action appears to exist between NMDA receptor antagonists and 5-HT_{2A} receptor agonists. Specifically, mTOR's signaling pathway may be modulated by 5-HT_{2A} receptor activation and NMDA antagonism. Without being bound by theory, such modulation of the mTOR pathway may underpin the immediate and long-lasting therapeutic and synergistic benefits of combined administration of both agents. As such, in some embodiments, administration of both agents at psychedelic or sub-psychedelic doses enables therapeutic efficacy without or minimizing psychiatric adverse effects.

In addition, it has been found that atrophy of neurons in the prefrontal cortex (PFC) plays a key role in the pathophysiology of depression and related disorders. The ability to promote both structural and functional plasticity in the PFC has been hypothesized to underlie the fast-acting antidepressant properties of the dissociative anesthetic ketamine but also the long-lasting effect after a single administration. Without being bound by theory, it is believed that the combination drug therapy disclosed herein may function by synergistically increasing neuritogenesis and spinogenesis, including increased density of dendritic spines, thereby providing or contributing to long-lasting therapeutic benefits.

A ratio of the 5-HT_{2A} receptor agonist and the NMDA receptor antagonist administered in the combination drug therapy may vary depending on the patient (i.e., subject), the identity of the active ingredient(s) selections of the combination, the dosage form(s), and the specific disease or condition being treated. It should be understood that a specific ratio of the combination for any particular patient will depend upon a variety of factors, such as the activity of the specific compounds employed for the 5-HT_{2A} receptor agonist and the NMDA receptor antagonist, the age, sex, general health of the patient, time of administration, rate of excretion, and the severity of the particular disease or condition being treated. In some embodiments, a weight ratio of the 5-HT_{2A} receptor agonist and the NMDA receptor antagonist administered to the patient may range from about 1:100 to about 100:1, or any range therebetween, e.g., from about 1:75, from about 1:50, from about 1:40, from about 1:30, from about 1:20, from about 1:10, from about 1:8, from about 1:6, from about 1:5, from about 1:4, from about 1:3, from about 1:2, from about 2:3, from about 1:1, and up to about 100:1, up to about 75:1, up to about 50:1, up to about 40:1, up to about 30:1, up to about 20:1, up to about 10:1, up to about 8:1, up to about 6:1, up to about 5:1, up to about 4:1, up to about 3:1, up to about 2:1. Ratios outside of this range may also be employed, in certain circumstances.

The combination drug therapy is intended to embrace administration of the 5-HT_{2A} receptor agonist and the NMDA receptor antagonist (e.g., nitrous oxide) in a sequential manner, that is, wherein each active ingredient is administered at a different time, as well as administration of these active ingredients, or at least two of the active ingredients, in a concurrent manner. Concurrent administration can be accomplished, for example, by administering to the subject a single dosage form having a fixed ratio of each active ingredient or in multiple, single dosage forms for each of the active ingredients. Administration of the 5-HT_{2A} receptor agonist and a NMDA receptor antagonist (e.g., nitrous oxide), whether in a single dosage form or separate dosage forms, can be carried out by any administration route set forth herein. In some embodiments, both the 5-HT_{2A} receptor agonist and the NMDA receptor antagonist are administered via inhalation, preferably in aerosol (e.g., mist) form. In some embodiments, the 5-HT_{2A} receptor agonist is administered intravenously (IV), and the NMDA receptor antagonist is administered via inhalation. In some embodiments, both the 5-HT_{2A} receptor agonist and the NMDA receptor antagonist are administered transdermally or subcutaneously. The compositions for inhalation such as pharmaceutically acceptable excipients, etc. for the single or separate dosage forms are set forth herein.

The present disclosure provides a combination drug therapy utilizing any one or more of the 5-HT_{2A} receptor agonists disclosed herein in combination with any one or more of the NMDA receptor antagonists disclosed herein. Examples of the combination drug therapy may include, but are not limited to, a compound of Formula (I) and nitrous oxide, a compound of Formula (II) and nitrous oxide, a compound of Formula (II-a) and nitrous oxide, a compound of Formula (II-b) and nitrous oxide, a compound of Formula (II-c) and nitrous oxide, a compound of Formula (II-d) and nitrous oxide, a compound of Formula (III) and nitrous oxide, a compound of Formula (III-a) and nitrous oxide, a compound of Formula (IV) and nitrous oxide, a compound of Formula (IV-a) and nitrous oxide, a compound of Formula (IV-b) and nitrous oxide, a compound of Formula (V) and nitrous oxide, a compound of Formula (V-a) and nitrous oxide, a compound of Formula (V-b) and nitrous oxide, a compound of Formula (VI) and nitrous oxide, a compound of Formula (VI-a) and nitrous oxide, a compound of Formula (VI-b) and nitrous oxide, a compound of Formula (I) and ketamine, a compound of Formula (II) and ketamine, a compound of Formula (II-a) and ketamine, a compound of Formula (II-b) and ketamine, a compound of Formula (I-c) and ketamine, a compound of Formula (II-d) and ketamine, a compound of Formula (III) and ketamine, a compound of Formula (III-a) and ketamine, a compound of Formula (IV) and ketamine, a compound of Formula (IV-a) and ketamine, a compound of Formula (IV-b) and ketamine, a compound of Formula (V) and ketamine, a compound of Formula (V-a) and ketamine, a compound of Formula (V-b) and ketamine, a compound of Formula (VI) and ketamine, a compound of Formula (VI-a) and ketamine, a compound of Formula (VI-b) and ketamine, including pharmaceutically acceptable salts, stereoisomers, or solvates of any compound in the combination.

Specific examples of the combination drug therapy may include, but are not limited to, psilocybin and nitrous oxide, psilocin and nitrous oxide, N,N-dimethyltryptamine (DMT) and nitrous oxide, 5-methoxy-N,N-dimethyltryptamine (5-MeO-DMT) and nitrous oxide, 2-(1H-indol-3-yl)-N,N-bis(methyl-d₃)ethan-1-amine-1,1,2,2-d₄ (DMT-d₁₀) and nitrous oxide, 2-(5-methoxy-1H-indol-3-yl)-N,N-bis(methyl-d₃)ethan-1-amine-1,1,2,2-d₄ (5-MeO-DMT-d₁₀) and nitrous oxide, psilocybin and ketamine, psilocin and ketamine, N,N-dimethyltryptamine (DMT) and ketamine, 5-methoxy-N,N-dimethyltryptamine (5-MeO-DMT) and ketamine, 2-(1H-indol-3-yl)-N,N-bis(methyl-d₃)ethan-1-amine-1,1,2,2-d₄ (DMT-d₁₀) and ketamine, and 2-(5-methoxy-1H-indol-3-yl)-N,N-bis(methyl-d₃)ethan-1-amine-1,1,2,2-d₄ (5-MeO-DMT-d₁₀) and ketamine, including pharmaceutically acceptable salts, stereoisomers, or solvates of any compound in the combination.

In the combination drug therapy disclosed herein, the 5-HT_{2A} receptor agonist and the NMDA receptor antagonist may be combined within a single molecule. Preferably, the 5-HT_{2A} receptor agonist and the NMDA receptor antagonist are combined via at least one linking agent. During treatment using such a single molecule, either the 5-HT_{2A} receptor agonist portion of the molecule binds to a 5-HT_{2A} receptor, the NMDA receptor antagonist portion of the molecule binds to an NMDA receptor, or both, to effect treatment.

In some embodiments, the 5-HT_{2A} receptor agonist and the NMDA receptor antagonist are combined as a pharmaceutically acceptable prodrug. As used herein, a “pharmaceutically acceptable prodrug” refers to a compound that is metabolized, for example hydrolyzed or oxidized, in the host to form the combination drug therapy of the present disclosure. Typical examples of prodrugs include compounds that have biologically labile protecting groups on a functional moiety of the active compound(s) (e.g., the 5-HT_{2A} receptor agonist and the NMDA receptor antagonist). Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound(s). An example, without limitation, of a prodrug would be a compound or a formulation containing the 5-HT_{2A} receptor agonist and the NMDA receptor antagonist combined via a chemical bond such as an ester, phosphate, amide, carbamate, or urea.

### Pharmaceutical Compositions

Also disclosed herein is a pharmaceutical composition for use in the combination drug therapy. The pharmaceutical composition may contain both the 5-HT_{2A} receptor agonist and the NMDA receptor antagonist in a single dosage form, or the 5-HT_{2A} receptor agonist and the NMDA receptor antagonist may be provided in separate pharmaceutical compositions. Typically, the pharmaceutical composition is also formulated with a pharmaceutically acceptable excipient.

A “pharmaceutical composition” refers to a mixture of the active ingredient(s) with other chemical components, such as pharmaceutically acceptable excipients. One purpose of a composition is to facilitate administration of the active ingredient(s) disclosed herein in any of its embodiments to a subject in need of combination drug therapy. In some embodiments, the 5-HT_{2A} receptor agonist and/or the NMDA receptor antagonist is/are the only active ingredient(s) present in the pharmaceutical composition.

The term “active ingredient”, as used herein, refers to an ingredient in the pharmaceutical composition that is biologically active, for example, one or more of the compounds described above as the 5-HT_{2A} receptor agonist, one or more of the compounds described above as the NMDA receptor antagonist, and any mixtures thereof. The 5-HT_{2A} receptor agonist and the NMDA receptor antagonist can be given per se or as a pharmaceutical composition containing the active ingredient(s) in combination with a pharmaceutically acceptable excipient. The pharmaceutical composition may contain at least 0.0001 wt. %, at least 0.001 wt. %, at least 0.01 wt. %, at least 0.05 wt. %, at least 0.1 wt. %, at least 0.5 wt. %, at least 5 wt. %, at least 10 wt. %, at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, at least 60 wt. %, at least 65 wt. %, at least 70 wt. %, at least 75 wt. %, at least 80 wt. %, at least 85 wt. %, at least 90 wt. %, at least 95 wt. %, at least 99 wt. %, or at least 99.9 wt. % of the 5-HT_{2A} receptor agonist and/or the NMDA receptor antagonist disclosed herein, relative to a total weight of the pharmaceutical composition.

The quantity of the 5-HT_{2A} receptor agonist and/or the NMDA receptor antagonist in a unit dose preparation may be varied or adjusted to provide (on active basis) e.g., from 0.001 mg, from 0.01 mg, from 0.1 mg, from 1 mg, from 3 mg, from 5 mg, from 10 mg, from 15 mg, from 20 mg, from 25 mg, and up to 100 mg, to 95 mg, to 90 mg, to 85 mg, to 80 mg, to 75 mg, to 70 mg, to 65 mg, to 60 mg, to 55 mg, to 50 mg, to 45 mg, to 40 mg, to 35 mg, to 30 mg of the 5-HT_{2A} receptor agonist and/or the NMDA receptor antagonist, or otherwise as deemed appropriate using sound medical judgment, according to the particular application, administration route, dosage form, potency of the active ingredient(s), etc. The composition can, if desired, also contain other compatible active ingredients.

In embodiments where the pharmaceutical composition is formulated with a deuterated 5-HT_{2A} receptor agonist, such as a compound of Formula (I), Formula (II), Formula (II-a), Formula (II-b), Formula (II-c), Formula (II-d), Formula (III), Formula (III-a), Formula (IV), Formula (IV-a), Formula (IV-b), Formula (V), Formula (V-a), Formula (V-b), Formula (VI), Formula (VI-a), or Formula (VI-b) comprising at least one deuterium atom, the pharmaceutical composition may comprise a single isotopologue or an isotopologue mixture of compounds, or pharmaceutically acceptable salts, solvates, or stereoisomers thereof. In some embodiments, a subject compound of Formula (I), Formula (II), Formula (II-a), Formula (II-b), Formula (II-c), Formula (II-d), Formula (III), Formula (III-a), Formula (IV), Formula (IV-a), Formula (IV-b), Formula (V), Formula (V-a), Formula (V-b), Formula (VI), Formula (VI-a), or Formula (VI-b) may be present in the pharmaceutical composition at a purity of at least 50% by weight, at least 60% by weight, at least 70% by weight, at least 80% by weight, at least 90% by weight, at least 95% by weight, at least 99% by weight, based on a total weight of isotopologues of the compound of Formula (I), Formula (II), Formula (II-a), Formula (II-b), Formula (II-c), Formula (II-d), Formula (III), Formula (III-a), Formula (IV), Formula (IV-a), Formula (IV-b), Formula (V), Formula (V-a), Formula (V-b), Formula (VI), Formula (VI-a), or Formula (VI-b) present in the pharmaceutical composition. For example, a pharmaceutical composition formulated with DMT-d₁₀, as the subject compound, may additionally contain isotopologues of the subject compound, e.g., DMT-d₉, a DMT-d₈, etc., as free-base or salt forms, stereoisomers, solvates, or mixtures thereof. In some embodiments, the composition is substantially free of other isotopologues of the compound, in either free base or salt form, e.g., the composition has less than 20, 15, 10, 9, 8, 7, 6, 5, 4, 3, 2 or 1 or 0.5 mole percent of other isotopologues of the compound.

In some embodiments, any position in the compound having deuterium has a minimum deuterium incorporation of at least 10 atom %, at least 20 atom %, at least 25 atom %, at least 30 atom %, at least 40 atom %, at least 45 atom %, at least 50 atom %, at least 60 atom %, at least 70 atom %, at least 80 atom %, at least 90 atom %, at least 95 atom %, at least 99 atom % at the site of deuteration.

The 5-HT_{2A} receptor agonist, and likewise, the NMDA receptor antagonist, may be present in the pharmaceutical composition in enantiomerically pure form, or as a racemic mixture. As described herein, a racemic active ingredient may contain about 50% of the R- and S-stereoisomers based on a molar ratio (about 48 to about 52 mol %, or about a 1:1 ratio)) of one of the isomers. In some embodiments, the pharmaceutical composition may be provided by combining separately produced compounds of the R- and S-stereoisomers in an approximately equal molar ratio (e.g., about 48 to 52%). In some embodiments, the pharmaceutical composition may contain a mixture of separate compounds of the R- and S-stereoisomers in different ratios. In some embodiments, the pharmaceutical composition contains an excess (greater than 50%) of the R-enantiomer. Suitable molar ratios of R/S may be from about 1.5:1, 2:1, 3:1, 4:1, 5:1, 10:1, or higher. In some embodiments, the pharmaceutical composition may contain an excess of the S-enantiomer, with the ratios provided for R/S reversed. Other suitable amounts of R/S may be selected. For example, the R-enantiomer may be enriched, e.g., may be present in amounts of at least about 55% to 100%, or at least 65%, at least 75%, at least 80%, at least 85%, at least 90%, about 95%, about 98%, or 100%. In some embodiments, the S-enantiomer may be enriched, e.g., in amounts of at least about 55% to 100%, or at least 65%, at least 75%, at least 80%, at least 85%, at least 90%, about 95%, about 98%, or 100%. Ratios between all these exemplary embodiments as well as greater than and less than them while still within the disclosure, all are included.

The 5-HT_{2A} receptor agonist and the NMDA receptor antagonist may be combined in a single pharmaceutical composition. In some embodiments, both the 5-HT_{2A} receptor agonist and the NMDA receptor antagonist (e.g., nitrous oxide) are administered together in a single pharmaceutical composition adapted for inhalation, preferably in aerosol (e.g., mist) form. In some embodiments, both the 5-HT_{2A} receptor agonist and the NMDA receptor antagonist (e.g., ketamine) are administered together in a single pharmaceutical composition adapted for transdermal or subcutaneous administration, for example, in a transdermal patch.

In some embodiments, the 5-HT_{2A} receptor agonist and the NMDA receptor antagonist are administered as separate pharmaceutical compositions. The 5-HT_{2A} receptor agonist may be formulated with a first pharmaceutically acceptable excipient to form a first pharmaceutical composition, and the NMDA receptor antagonist may be formulated with a second pharmaceutically acceptable excipient to form a second pharmaceutical composition. The first composition comprising the 5-HT_{2A} receptor agonist and the second composition comprising the NMDA receptor antagonist may be administered concurrently or sequentially. In some embodiments, the first pharmaceutical composition containing the 5-HT_{2A} receptor agonist (e.g., DMT, 5-MeO-DMT, DMT-d₁₀, 5-MeO-DMT-d₁₀, etc.) is adapted for parenteral delivery such as intravenous administration, and the second pharmaceutical composition containing the NMDA receptor antagonist (e.g., nitrous oxide) is adapted for inhalation administration such as a therapeutic gas mixture.

In some embodiments, the 5-HT_{2A} receptor agonist and the NMDA receptor antagonist are formulated separately but are combined into a single pharmaceutical composition just prior to administration. In one non-limiting example, the 5-HT_{2A} receptor agonist may be formulated as a solution, while the NMDA receptor antagonist (e.g., nitrous oxide) may formulated in a therapeutic gas mixture. An aerosol, preferably a mist, may then be generated containing liquid droplets of the 5-HT_{2A} receptor agonist dissolved in solution, the liquid droplets being dispersed in a gas phase of the therapeutic gas mixture containing the NMDA receptor antagonist. The aerosol, combining both the 5-HT_{2A} receptor agonist and the NMDA receptor antagonist, may then be administered to the patient via inhalation. In another non-limiting example, the 5-HT_{2A} receptor agonist may be formulated as a solution, while the NMDA receptor antagonist (e.g., nitrous oxide) may formulated in a therapeutic gas mixture. An aerosol, preferably a mist, may then be generated containing liquid droplets of the 5-HT_{2A} receptor agonist dissolved in solution, the liquid droplets being dispersed in a gas phase of e.g., a heated heliox mixture. The aerosol containing the 5-HT_{2A} receptor agonist dispersed in the gas phase of the heated heliox mixture may then be combined with the therapeutic gas mixture containing the NMDA receptor antagonist, for administration to the patient via inhalation.

“Pharmaceutically acceptable excipients” may be excipients approved by a regulatory agency of the Federal or a state government or listed in the U.S. Pharmacopeia or other generally recognized pharmacopeia for use in mammals, such as humans. The term “excipient” herein refers to a vehicle, diluent, adjuvant, carrier, or any other auxiliary or supporting ingredient with which the 5-HT_{2A} receptor agonist and/or the NMDA receptor antagonist of the present disclosure is formulated for administration to a mammal. Such pharmaceutical excipients can be liquids, such as water and oils, including those of petroleum, animal, vegetable or synthetic origin, such as peanut oil, soybean oil, mineral oil, sesame oil and the like. The pharmaceutical excipients can be water, saline, gum acacia, gelatin, starch paste, talc, keratin, colloidal silica, urea, and the like. The pharmaceutical excipients can include one or more gases, e.g., to act as a carrier for administration via inhalation. In addition, auxiliary, stabilizing, thickening, lubricating, taste masking, coloring agents, and other pharmaceutical additives may be included in the disclosed compositions, for example those set forth hereinafter. In some embodiments, the pharmaceutical acceptable excipient is a carrier useful for administration via inhalation. In some embodiments, the pharmaceutically acceptable excipient is an aerosol carrier, which will be described in more detail further below. In some embodiments, the pharmaceutically acceptable excipient is useful for parenteral administration, such as via intravenous administration. In some embodiments, the pharmaceutically acceptable excipient is useful for transdermal or subcutaneous administration.

In some embodiments, the pharmaceutical composition contains 0.1 to 99.9999 wt. %, preferably 1 to 99.999 wt. %, preferably 5 to 99.99 wt. %, preferably 10 to 99.9 wt. %, preferably 15 to 99 wt. %, preferably 20 to 90 wt. %, preferably 30 to 85 wt. %, preferably 40 to 80 wt. %, preferably 50 to 75 wt. %, preferably 60 to 70 wt. % of the pharmaceutically acceptable excipient relative to a total weight of the pharmaceutical composition.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical excipients and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference. The choice of excipient will be determined in part by the particular active ingredient(s), as well as by the particular method used to administer the composition. Accordingly, there is a wide variety of suitable formulations of the pharmaceutical compositions. Liquid form preparations include solutions and emulsions, for example, water, water/propylene glycol solutions, or organic solvents. When administered to a mammal, the compounds and compositions of the present disclosure and pharmaceutically acceptable excipients may be sterile. In some instances, an aqueous medium is employed as a vehicle e.g., when the subject compound is administered intravenously or via inhalation, such as water, saline solutions, and aqueous dextrose and glycerol solutions.

As described below, the pharmaceutical compositions of the present disclosure may be specially formulated for administration in solid, semi-solid, or liquid form, including those adapted for the following:
A. Oral administration, for example, drenches (aqueous or non-aqueous solutions or suspensions), tablets, films, or capsules, e.g., those targeted for buccal, sublingual, and systemic absorption, boluses, powders, granules, syrups, pastes for application to the tongue;
B . Parenteral administration, for example, by subcutaneous, intramuscular, intravenous or epidural injection as, for example, a sterile solution or suspension, or sustained release formulation;
C. Topical application/transdermal administration, for example, as a cream, ointment, or a controlled release patch or spray applied to the skin, or orifices and/or mucosal surfaces such as intravaginally or intrarectally, for example, as a pessary, cream or foam;
D. Modified release dosage forms, including delayed-, extended-, prolonged-, sustained-, pulsatile-, controlled-, accelerated-, fast-, targeted-, programmed-release, and gastric retention dosage forms, such modified release dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126); and
E. Inhalation administration, for example as an aerosol, preferably a mist.

Tamper resistant dosage forms/packaging of any of the disclosed pharmaceutical compositions are contemplated.

### A. Oral Administration

The pharmaceutical compositions disclosed herein may be provided in solid, semisolid, or liquid dosage forms for oral administration, including both enteric/gastric delivery routes as well as intraoral routes such as buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remains intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof.

Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions disclosed herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain e.g., from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; tale; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions disclosed herein may contain e.g., about 0.1 to about 5% by weight of a lubricant.

Suitable glidants include colloidal silicon dioxide, CAB-O-SIL® (Cabot Co. of Boston, Mass.), and asbestos-free tale. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many excipients may serve several functions, even within the same formulation.

The pharmaceutical compositions disclosed herein may be formulated as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredient(s) from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient(s) in powdered, crystalline, or granular forms, alone or in combination with one or more excipients described herein, including binders, disintegrants, controlled-release polymers, lubricants, diluents, and/or colorants. Flavoring and sweetening agents are especially useful in the formation of chewable tablets and lozenges.

The pharmaceutical compositions disclosed herein may be formulated as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient(s). The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl— and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient(s).

In some embodiments, pharmaceutical compositions of the present disclosure may be in orodispersible dosage forms (ODxs), including orally disintegrating tablets (ODTs) (also sometimes referred to as fast disintegrating tablets, orodispersible tablets, or fast dispersible tablets) or orodispersible films (ODFs) (or wafers). Such dosage forms allow for pre-gastric absorption of the active ingredient(s), e.g., when administered intraorally/transmucosally through the mucosal linings of the oral cavity, e.g., buccal, lingual, and sublingual administration, for increased bioavailability and faster onset compared to oral administration through the gastrointestinal tract.

Orally disintegrating tablets can be prepared by different techniques, such as freeze drying (lyophilization), molding, spray drying, mass extrusion or compressing. Preferably, the orally disintegrating tablets are prepared by lyophilization. In some embodiments, orally disintegrating tablet refers to forms which disintegrate in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, orally disintegrating tablet refers to forms which dissolve in less than about 90 seconds, in less than about 60 seconds, or in less than about 30 seconds after being received in the oral cavity. In some embodiments, orally disintegrating tablet refers to forms which disperse in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, the pharmaceutical compositions are in the form of orodispersible dosage forms, such as oral disintegrating tablets (ODTs), having a disintegration time according to the United States Phamacopeia (USP) disintegration test <701> of not more than about 30 seconds, not more than about 20, not more than about 10 seconds, not more than about 5 seconds, not more than about 2 seconds. Orodispersible dosage forms having longer disintegration times according to the United States Phamacopeia (USP) disintegration test <701>, such as when adapted for extended release, for example on the order of 30 minutes or less, 20 minutes or less, 10 minutes or less, 5 minutes or less, 4 minutes or less, 3 minutes or less, 2 minutes or less, are also contemplated.

In some embodiments, the pharmaceutical compositions are in the form of lyophilized orodispersible dosage forms, such as lyopholized ODTs. In some embodiments, the lyophilized orodispersible dosage forms (e.g., lyophilized ODTs) are created by creating a porous matrix by subliming the water from pre-frozen aqueous formulation of the drug containing matrix-forming agents and other excipients such as those set forth herein, e.g., one or more lyoprotectants, preservatives, antioxidants, stabilizing agents, solubilizing agents, flavoring agents, etc. In some embodiments, the orodispersible dosage forms comprise two component frameworks of a lyophilized matrix system that work together to ensure the development of a successful formulation. In some embodiments, the first component is a water-soluble polymer such as gelatin, dextran, alginate, and maltodextrin. This component maintains the shape and provides mechanical strength to the dosage form (binder). In some embodiments, the second constituent is a matrix-supporting/disintegration-enhancing agent such as sucrose, lactose, mannitol, xylitol, microcrystalline cellulose, calcium diphosphate, and/or starch, which acts by cementing the porous framework, provided by the water-soluble polymer and accelerates the disintegration of the orodispersible dosage forms. In some embodiments, the lyophilized orodispersible dosage form (e.g., lyophilized ODT) includes gelatin and mannitol. In some embodiments, the lyophilized orodispersible dosage form (e.g., lyophilized ODT) includes gelatin, mannitol, and one or more of a lyoprotectant, a preservative, an antioxidant, a stabilizing agent, a solubilizing agent, a flavoring agent, etc., with particular mention being made to citric acid. A non-limiting example of an ODT formulation is Zydis® orally dispersible tablets (available from Catalent). In some embodiments, the ODT formulation (e.g., Zydis® orally dispersible tablets) includes one or more water-soluble polymers, such as gelatin, one or more matrix materials, fillers, or diluents, such as mannitol, an active ingredient(s), and optionally a lyoprotectant, a preservative, an antioxidant, a stabilizing agent, a solubilizing agent, and/or a flavoring agent. In some embodiments, the ODT formulation (e.g., Zydis® orally dispersible tablets) includes gelatin, mannitol, an active ingredient(s), and citric acid and/or tartaric acid.

In some embodiments, the pharmaceutical compositions are in the form of lyophilized orodispersible films (ODFs) (or wafers). In some embodiments, the pharmaceutical compositions are in the form of lyophilized ODFs protected for the long-term storage by a specialty packaging excluding moisture, oxygen, and light. In some embodiments, the lyophilized ODFs are created by creating a porous matrix by subliming the water from pre-frozen aqueous formulation of the drug containing matrix-forming agents and other vehicles such as those set forth herein, e.g., one or more of a lyoprotectant, a preservative, an antioxidant, a stabilizing agent, a solubilizing agent, a flavoring agent, etc. In some embodiments, the lyophilized ODF includes a thin water-soluble film matrix. In some embodiments, the ODFs comprise two component frameworks of a lyophilized matrix system that work together to ensure the development of a successful formulation. In some embodiments, the first component is water-soluble polymers such as gelatin, dextran, alginate, and maltodextrin. This component maintains the shape and provides mechanical strength to the film/wafer (binder). In some embodiments, the second constituent is matrix-supporting/disintegration-enhancing agents such as sucrose and mannitol, which acts by cementing the porous framework, provided by the water-soluble polymer and accelerates the disintegration of the wafer. In some embodiments, the lyophilized ODFs include gelatin and mannitol. In some embodiments, the lyophilized ODFs include gelatin, mannitol, and one or more of a lyoprotectant, a preservative, an antioxidant, a stabilizing agent, a solubilizing agent, a flavoring agent, etc., with particular mention being made to citric acid.

In some embodiments, the ODF (or wafer) can comprise a monolayer, bilayer, or trilayer. In some embodiments, the monolayer ODF contains an active ingredient(s) and one or more pharmaceutically acceptable excipients. In some embodiments, the bilayer ODE contains one or more excipients, such as a solubilizing agent, in a first layer and an active ingredient(s) in the second layer. This configuration allows the active ingredient(s) to be stored separately from the excipients and can increase the stability of the active ingredient(s) and optionally increase the shelf life of the composition compared to the case where the excipients and the active ingredient(s) were contained in a single layer. For tri-layer ODFs, each of the layers may be different or two of the layers, such as the upper and lower layers, may have substantially the same composition. In some embodiments, the lower and upper layers surround a core layer containing the active ingredient(s). In some embodiments, the lower and upper layers may contain one or more excipients, such as a solubilizing agent. In some embodiments, the lower and upper layers have the same composition. Alternatively, the lower and upper layers may contain different excipients or different amounts of the same excipient. The core layer typically contains the active ingredient(s), optionally with one or more excipients.

In some embodiments, in addition to the active ingredient(s), the pharmaceutical compositions in orodispersible dosage forms (ODxs) may contain one or more pharmaceutically acceptable excipients. For example, in some embodiments, pharmaceutical compositions in orodispersible dosage forms include one or more of pharmaceutically acceptable a lyoprotectant, a preservative, an antioxidant, a stabilizing agent, a solubilizing agent, a flavoring agent, etc.

Examples of pharmaceutically acceptable lyoprotectants include, but are not limited to, disaccharides such as sucrose and trehalose, anionic polymers such as sulfobutylether-β-cyclodextrin (SBECD) and hyaluronic acid, and hydroxylated cyclodextrins.

Examples of pharmaceutically acceptable preservatives include, but are not limited to, glycerin, methyl and propylparaben, benzoic acid, sodium benzoate and alcohol.

Examples of pharmaceutically acceptable antioxidants, which may act to further enhance stability of the composition, include: (1) water soluble antioxidants, such as ascorbic acid, cysteine or salts thereof (cysteine hydrochloride), sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of pharmaceutically acceptable stabilizing agents include, but are not limited to, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, glycerol, methionine, monothioglycerol, ascorbic acid, citric acid, polysorbate, arginine, cyclodextrins, microcrystalline cellulose, modified celluloses (e.g., carboxymethylcellulose, sodium salt), sorbitol, and cellulose gel.

Examples of pharmaceutically acceptable solubilizing agents (or dissolution aids) include, but are not limited to, citric acid, hydroxypropylcellulose, hydroxypropylmethylcellulose, sodium stearyl fumarate, methacrylic acid copolymer LD, methylcellulose, sodium lauryl sulfate, polyoxyl 40 stearate, purified shellac, sodium dehydroacetate, fumaric acid, DL-malic acid, L-ascorbyl stearate, L-asparagine acid, adipic acid, aminoalkyl methacrylate copolymer E, propylene glycol alginate, casein, casein sodium, a carboxyvinyl polymer, carboxymethylethylcellulose, powdered agar, guar gum, succinic acid, copolyvidone, cellulose acetate phthalate, tartaric acid, dioctylsodium sulfosuccinate, zein, powdered skim milk, sorbitan trioleate, lactic acid, aluminum lactate, ascorbyl palmitate, hydroxyethylmethylcellulose, hydroxypropylmethylcelluloseacetate succinate, polyoxyethylene (105) polyoxypropylene (5) glycol, polyoxyethylene hydrogenated castor oil 60, polyoxyl 35 castor oil, poly(sodium 4-styrenesulfonate), polyvinylacetaldiethylamino acetate, polyvinyl alcohol, maleic acid, methacrylic acid copolymer S, lauromacrogol, sulfuric acid, aluminum sulfate, phosphoric acid, calcium dihydrogen phosphate, sodium dodecylbenzenesulfonate, a vinyl pyrrolidone-vinyl acetate copolymer, sodium lauroyl sarcosinate, acetyl tryptophan, sodium methyl sulfate, sodium ethyl sulfate, sodium butyl sulfate, sodium octyl sulfate, sodium decyl sulfate, sodium tetradecyl sulfate, sodium hexadecyl sulfate, and sodium octadecyl sulfate. Of these, in some embodiments, such as in ODT formulation, citric acid is preferred.

Flavoring agents include natural flavors extracted from plants, such as fruits, and synthetic blends of compounds which produce a pleasant taste sensation or taste masking effect. Examples of flavoring agents include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, glucose, wild orange peel, citric acid, tartaric acid, oil of wintergreen, oil of peppermint, methyl salicylate, oil of spearmint, oil of sassafras, oil of clove, cinnamon, anethole, menthol, thymol, eugenol, eucalyptol, lemon, lime, and lemon-lime.

Cyclodextrins such as α-cyclodextrin, β-cyclodextrin, γ-cyclodextrin, methyl-β-cyclodextrin, hydroxyethyl β-cyclodextrin, hydroxypropyl-β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

Disclosed herein are pharmaceutical compositions in modified release dosage forms, which comprise an active ingredient(s) as disclosed herein and one or more release controlling excipients or carriers as described herein. Suitable modified release dosage excipients include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients or carriers.

Further disclosed herein are pharmaceutical compositions in enteric coated dosage forms, which comprise a compound as disclosed herein and one or more release controlling excipients or carriers for use in an enteric coated dosage form. The pharmaceutical compositions may also comprise non-release controlling excipients or carriers.

Further disclosed herein are pharmaceutical compositions in effervescent dosage forms, which comprise an active ingredient(s) as disclosed herein and one or more release controlling excipients or carriers for use in an effervescent dosage form. The pharmaceutical compositions may also comprise non-release controlling excipients or carriers.

Additionally, disclosed are pharmaceutical compositions in a dosage form that has an instant releasing component and at least one delayed releasing component, and is capable of giving a discontinuous release of the active ingredient(s) in the fora of at least two consecutive pulses separated in time from about 0.1 up to about 24 hours (e.g., about 0.1, 0.5, 1, 2, 4, 6, 8, 10, 12, 14, 16, 18, 10, 22, or 24 hours). The pharmaceutical compositions comprise the 5-HT_{2A} receptor agonist and/or the NMDA receptor antagonist as disclosed herein and one or more release controlling and non-release controlling excipients or carriers, such as those excipients or carriers suitable for a disruptable semipermeable membrane and as swellable substances.

Disclosed herein also are pharmaceutical compositions in a dosage form for oral administration to a subject, which comprise the 5-HT_{2A} receptor agonist and/or the NMDA receptor antagonist as disclosed herein and one or more pharmaceutically acceptable excipients, enclosed in an intermediate reactive layer comprising a gastric juice-resistant polymeric layered material partially neutralized with alkali and having cation exchange capacity and a gastric juice-resistant outer layer.

In some embodiments, the pharmaceutical compositions are in the form of immediate-release capsules for oral administration, and may further comprise cellulose, iron oxides, lactose, magnesium stearate, and sodium starch glycolate.

In some embodiments, the pharmaceutical compositions are in the form of delayed-release capsules for oral administration, and may further comprise cellulose, ethylcellulose, gelatin, hypromellose, iron oxide, and titanium dioxide.

In some embodiments, the pharmaceutical compositions are in the form of enteric coated delayed-release tablets for oral administration, and may further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In some embodiments, the pharmaceutical compositions are in the form of enteric coated delayed-release tablets for oral administration, and may further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The pharmaceutical compositions disclosed herein may be formulated as liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and optional preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates. In some embodiments, examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl pahmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Cyclodextrins such as α-cyclodextrin, β-cyclodextrin, γ-cyclodextrin, hydroxyethyl β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

The pharmaceutical compositions disclosed herein for oral administration may be also disclosed in the forms of liposomes, micelles, microspheres, or nanosystems.

The pharmaceutical compositions disclosed herein may be disclosed as non-effervescent or effervescent, granules and powders, to be reconstituted into a liquid dosage form. Pharmaceutically acceptable excipients used in the non-effervescent granules or powders may include diluents, sweeteners, and wetting agents. Pharmaceutically acceptable excipients used in the effervescent granules or powders may include organic acids and a source of carbon dioxide.

Coloring and flavoring agents can be used in all of the above dosage forms.

The pharmaceutical compositions disclosed herein may be co-formulated with other active ingredients which do not impair the desired therapeutic action, or with substances that supplement the desired action.

### B. Parenteral Administration

The pharmaceutical compositions disclosed herein may be administered parenterally by injection, infusion, perfusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including ca-cyclodextrin, β-cyclodextrin, hydroxypropyl-3-cyclodextrin, sulfobutylether-β-cyclodextrin, and sulfobutylether 7-O-cyclodextrin (CAPTISOL®, CyDex, Lenexa, Kans.).

The pharmaceutical compositions disclosed herein may be formulated for single or multiple dosage administration. The single dosage formulations are packaged in an ampule, a vial, or a syringe. The multiple dosage parenteral formulations contain an antimicrobial agent at bacteriostatic or fungistatic concentrations. All parenteral formulations must be sterile, as known and practiced in the art.

In some embodiments, the pharmaceutical compositions are disclosed as ready-to-use sterile solutions. In some embodiments, the pharmaceutical compositions are disclosed as sterile dry soluble products, including lyophilized powders and hypodermic tablets, to be reconstituted with a vehicle prior to use. In some embodiments, the pharmaceutical compositions are disclosed as ready-to-use sterile suspensions. In some embodiments, the pharmaceutical compositions are disclosed as sterile dry insoluble products to be reconstituted with a vehicle prior to use. In some embodiments, the pharmaceutical compositions are disclosed as ready-to-use sterile emulsions.

The pharmaceutical compositions may be formulated as a suspension, solid, semi-solid, or thixotropic liquid, for administration as an implanted depot. In some embodiments, the pharmaceutical compositions disclosed herein are dispersed in a solid inner matrix, which is surrounded by an outer polymeric membrane that is insoluble in body fluids but allows the active ingredient(s) in the pharmaceutical compositions to diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate, and the like.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, and the like.

### C. Topical Administration

The pharmaceutical compositions disclosed herein may be administered topically to the skin, orifices, or mucosa. Topical administration, as described herein, includes (intra)dermal, conjuctival, intracoreal, intraocular, ophthalmic, auricular, transdermal, nasal, vaginal, uretheral, respiratory, and rectal administration.

The pharmaceutical compositions disclosed herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may contain the active ingredient(s) which may be mixed under sterile conditions with a pharmaceutically acceptable excipient, and with any preservatives, buffers, absorption enhancers, propellants which may be required. Liposomes, micelles, microspheres, nanosystems, and mixtures thereof, may also be used.

Pharmaceutically acceptable excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The ointments, pastes, creams and gels may contain, in addition to an active ingredient(s), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal delivery devices (e.g., patches) have the added advantage of providing controlled delivery of active ingredient(s) to the body. That is, the 5-HT_{2A} receptor agonist and/or the NMDA receptor antagonist of the present disclosure can be administered via a transdermal patch at a steady state concentration, whereby the active ingredient(s) is gradually administered over time, thus avoiding drug spiking and adverse events/toxicity associated therewith.

Transdermal patch dosage forms herein may be formulated with various amounts of the active ingredient(s), depending on the disease/condition being treated, the active ingredient(s) employed, the permeation and size of the transdermal delivery device, the release time period, etc. For example, when formulated with a 5-HT_{2A} receptor agonist, a unit dose preparation may be varied or adjusted e.g., from 5 mg, 10 mg, 15 mg, 20 mg, 25 mg, 30 mg, 35 mg, 40 mg, 45 mg, 50 mg, to 100 mg, 95 mg, 90 mg, 85 mg, 80 mg, 75 mg, 70 mg, 65 mg, 60 mg, 55 mg, or otherwise as deemed appropriate using sound medical judgment, according to the particular application and the potency of the 5-HT_{2A} receptor agonist. In another example, when formulated with a NMDA receptor antagonist (e.g., ketamine), a unit dose preparation may be varied or adjusted e.g., from 5 mg, 10 mg, 20 mg, 30 mg, 40 mg, 50 mg, 60 mg, 70 mg, 80 mg, 90 mg, 100 mg, to 5,000 mg, 4,000 mg, 3,000 mg, 2,000 mg, 1,000 mg, 900 mg, 800 mg, 700 mg, 600 mg, 500 mg, 400 mg, 300 mg, 200 mg, or otherwise as deemed appropriate using sound medical judgment, according to the particular application and the potency of the NMDA receptor antagonist.

Transdermal patches formulated with the disclosed 5-HT_{2A} receptor agonist and/or the NMDA receptor antagonist may be suitable for microdosing to achieve durable therapeutic benefits, with decreased toxicity. In some embodiments, the 5-HT_{2A} receptor agonist and/or the NMDA receptor antagonist of the present disclosure may be administered via a transdermal patch at serotonergic, but sub-psychoactive concentrations, for example, over an extended period such as over a 8, 24, 48, 72, 84, 96, or 168 hour time period.

In addition to the active ingredient(s) (i.e., 5-HT_{2A} receptor agonist and/or the NMDA receptor antagonist) and any optional pharmaceutically acceptable excipient(s), the transdermal patch may also include one or more of a pressure sensitive adhesive layer, a backing, and a release liner, as is known to those of ordinary skill in the art.

Transdermal patch dosage forms can be made by dissolving or dispersing the 5-HT_{2A} receptor agonist and/or the NMDA receptor antagonist in the proper medium. In some embodiments, the 5-HT_{2A} receptor agonist and/or the NMDA receptor antagonist of the present disclosure may be dissolved/dispersed directly into a polymer matrix forming the pressure sensitive adhesive layer. Such transdermal patches are called drug-in-adhesive (DIA) patches. Preferred DIA patch forms are those in which the active ingredient(s) is distributed uniformly throughout the pressure sensitive adhesive polymer matrix. In some embodiments, the active ingredient(s) may be provided in a layer containing the active ingredient(s) plus a polymer matrix which is separate from the pressure sensitive adhesive layer. In any case, the 5-HT_{2A} receptor agonist and/or the NMDA receptor antagonist of the present disclosure may optionally be formulated with suitable excipient(s) such as carriers, permeation agents/absorption enhancers, humectants, etc. to increase the flux across the skin.

Examples of carrier agents may include, but are not limited to, C₈-C₂₂ fatty acids, such as oleic acid, undecanoic acid, valeric acid, heptanoic acid, pelargonic acid, capric acid, lauric acid, and eicosapentaenoic acid; C₈-C₂₂ fatty alcohols such as octanol, nonanol, oleyl alcohol, decyl alcohol and lauryl alcohol; lower alkyl esters of C₈-C₂₂ fatty acids such as ethyl oleate, isopropyl myristate, butyl stearate, and methyl laurate; di(lower)alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; glycerol; ethyl acetate; acetoacetic ester; N-alkylpyrrolidone; cyclodextrins, such as α-cyclodextrin, β-cyclodextrin, γ-cyclodextrin, or derivatives such as 2-hydroxypropyl-β-cyclodextrin; and terpenes/terpenoids, such as limonene, linalool, myrcene, pinene such as α-pinene, caryophyllene, citral, eucolyptol, and the like; including mixtures thereof.

Examples of permeation agents/absorption enhancers include, but are not limited to, sulfoxides, such as dodecylmethylsulfoxide, octyl methyl sulfoxide, nonyl methyl sulfoxide, decyl methyl sulfoxide, undecyl methyl sulfoxide, 2-hydroxydecyl methyl sulfoxide, 2-hydroxy-undecyl methyl sulfoxide, 2-hydroxydodecyl methyl sulfoxide, and the like; surfactant-lecithin organogel (PLO), such as those formed from an aqueous phase with one or more of poloxamers, CARBOPOL and PEMULEN, a lipid phase formed from one or more of isopropyl palmitate and PPG-2 myristyl ether propionate, and lecithin; fatty acids, esters, and alcohols, such as oleyloleate and oleyl alcohol; keto acids such as levulinic acid; glycols and glycol ethers, such as diethylene glycol monoethyl ether; including mixtures thereof.

Examples of humectants/crystallization inhibitors include, but are not limited to, polyvinyl pyrrolidone-co-vinyl acetate, polymethacrylate, and mixtures thereof.

The pressure sensitive adhesive layer may be formed from polymers including, but not limited to, acrylics (polyacrylates including alkyl acrylics), polyvinyl acetates, natural and synthetic rubbers (e.g., polyisobutylene), ethylenevinylacetate copolymers, polysiloxanes, polyurethanes, plasticized polyether block amide copolymers, plasticized styrene-butadiene rubber block copolymers, and mixtures thereof. The pressure-sensitive adhesive layer used in the transdermal patch of the present disclosure may be formed from an acrylic polymer pressure-sensitive adhesive, preferably an acrylic copolymer pressure sensitive adhesive. The acrylic copolymer pressure sensitive adhesive may be obtained by copolymerization of one or more alkyl (meth)acrylates (e.g., 2-ethylhexyl acrylate); aryl (meth)acrylates; arylalkyl (meth)acrylate; and (meth)acrylates with functional groups such as hydroxyalkyl (meth)acrylates (e.g., hydroxyethyl acrylate, 2-hydroxypropyl acrylate, 3-hydroxypropyl acrylate, 4-hydroxybutyl acrylate, 2-hydroxyethyl methacrylate, 2-hydroxypropyl methacrylate, 3-hydroxypropyl methacrylate, and 4-hydroxybutyl methacrylate), carboxylic acid containing (meth)acrylates (e.g., acrylic acid), and alkoxy (meth)acrylates (e.g., methoxyethyl acrylate); optionally with one or more copolymerizable monomers (e.g., vinylpyrrolidone, vinyl acetate, etc.). Specific examples of acrylic pressure-sensitive adhesives may include, but are not limited to, DURO-TAK products (Henkel) such as DURO-TAK 87-900A, DURO-TAK 87-9301, DURO-TAK 87-4098, DURO-TAK 87-2074, DURO-TAK 87-235A, DURO-TAK 87-2510, DURO-TAK 87-2287, DURO-TAK 87-4287, DURO-TAK 87-2516, DURO-TAK 387-2052, and DURO-TAK 87-2677.

The backing used in the transdermal patch of the present disclosure may include flexible backings such as films, nonwoven fabrics, Japanese papers, cotton fabrics, knitted fabrics, woven fabrics, and laminated composite bodies of a nonwoven fabric and a film. Such a backing is preferably composed of a soft material that can be in close contact with a skin and can follow skin movement and of a material that can suppress skin rash and other discomforts following prolonged use of the patch. Examples of the backing materials include, but are not limited to, polyethylene, polypropylene, polyethylene terephthalate, polybutylene terephthalate, polyethylene naphthalate, polystyrene, nylon, cotton, acetate rayon, rayon, a rayon/polyethylene terephthalate composite body, polyacrylonitrile, polyvinyl alcohol, acrylic polyurethane, ester polyurethane, ether polyurethane, a styrene-isoprene-styrene copolymer, a styrene-butadiene-styrene copolymer, a styrene-ethylene-propylene-styrene copolymer, styrene-butadiene rubber, an ethylene-vinyl acetate copolymer, or cellophane, for example. Preferred backings do not adsorb or release the active ingredient(s). In order to suppress the adsorption and release of the active ingredient(s), to improve transdermal absorbability of the active ingredient(s), and to suppress skin rash and other discomforts, the backing preferably includes one or more layers composed of the material above and has a water vapor permeability. Specific examples of backings may include, but are not limited to, 3M COTRAN products such as 3M COTRAN ethylene vinyl acetate membrane film 9702, 3M COTRAN ethylene vinyl acetate membrane film 9716, 3M COTRAN polyethylene membrane film 9720, 3M COTRAN ethylene vinyl acetate membrane film 9728, and the like.

The release liner used in the transdermal patch of the present disclosure may include, but is not limited to, a polyester film having one side or both sides treated with a release coating, a polyethylene laminated high-quality paper treated with a release coating, and a glassine paper treated with a release coating. The release coating may be a fluoropolymer, a silicone, a fluorosilicone, or any other release coating known to those of ordinary skill in the art. The release liner may have an uneven surface in order to easily take out the transdermal patch from a package.

Examples of release liners may include, but are not limited to SCOTCHPAK products from 3M such as 3M SCOTCHPAK 9744, 3M SCOTCHPAK 9755, 3M SCOTCHPAK 9709, and 3M SCOTCHPAK 1022.

Other layers such as abuse deterrent layers formulated with one or more irritants (e.g., sodium lauryl sulfate, poloxamer, sorbitan monoesters, glyceryl monooleates, spices, etc.), may also be employed.

Methods disclosed herein using a transdermal patch dosage form provide for systemic delivery of small doses of active ingredient(s), preferably over extended periods of time such as up to 168 hour time periods, for example from 2 to 96 hours, or 4 to 72 hours, or 8 to 24 hours, or 10 to 18 hours, or 12 to 14 hours. In particular, the 5-HT_{2A} receptor agonist and/or the NMDA receptor antagonist of the present disclosure can be delivered in small, steady, and consistent doses such that deleterious or undesirable side-effects can be avoided. In some embodiments, the 5-HT_{2A} receptor agonist and/or the NMDA receptor antagonist of the present disclosure are administered transdermally at serotonergic, but sub-psychoactive concentrations.

Therefore, provided herein are methods of treating a disease or disorder associated with a serotonin 5-HT₂ receptor, such as a central nervous system (CNS) disorder, a psychological disorder, or an autonomic nervous system (ANS), or a disease or disorder modulated by N-methyl-D-aspartic acid (NMDA) activity, comprising administering the 5-HT_{2A} receptor agonist and/or the NMDA receptor antagonist via a transde al patch. Here, the 5-HT_{2A} receptor agonist and/or the NMDA receptor antagonist is capable of diffusing from the matrix of the transdermal patch (e.g., from the pressure sensitive adhesive layer) across the skin of the subject and into the bloodstream of the subject.

An exemplary drug-in-adhesive (DIA) patch formulation may comprise 5 to 30 wt. % NMDA receptor antagonist (e.g., ketamine), 5 to 30 wt. % 5-HT_{2A} receptor agonist (DMT, DMT-d₁₀ etc.), 30 to 70 wt. % pressure sensitive adhesive (e.g., DURO-TAK 387-2052, DURO-TAK 87-2677, and DURO-TAK 87-4098), 1 to 10 wt. % permeation agents/absorption enhancers (e.g., oleyloleate, oleyl alcohol, levulinic acid, diethylene glycol monoethyl ether, etc.), and 5 to 25 wt. % crystallization inhibitor (e.g., polyvinyl pyrrolidone-co-vinyl acetate, polymethacrylate, etc.), each based on a total weight of the DIA patch formulation, though it should be understood that many variations are possible in light of the teachings herein.

Automatic injection devices offer a method for delivery of the compositions disclosed herein to patients. The compositions disclosed herein may be administered to a patient using automatic injection devices through a number of known devices, a non-limiting list of which includes transdermal, subcutaneous, and intramuscular delivery.

In some transdermal, subcutaneous, or intramuscular applications, a composition disclosed herein is absorbed through the skin. Passive transdermal patch devices often include an absorbent layer or membrane that is placed on the outer layer of the skin. The membrane typically contains a dose of a substance that is allowed to be absorbed through the skin to deliver the composition to the patient. Typically, only substances that are readily absorbed through the outer layer of the skin may be delivered with such transdermal patch devices.

Other automatic injection devices disclosed herein are configured to provide for increased skin permeability to improve delivery of the disclosed compositions. Non-limiting examples of structures used to increase permeability to improve transfer of a composition into the skin, across the skin, or intramuscularly include the use of one or more microneedles, which in some embodiments may be coated with a composition disclosed herein. Alternatively, hollow microneedles may be used to provide a fluid channel for delivery of the disclosed compositions below the outer layer of the skin. Other devices disclosed herein include transdermal delivery by iontophoresis, sonophoresis, reverse iontophoresis, or combinations thereof, and other technologies known in the art to increase skin permeability to facilitate drug delivery.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions disclosed herein may be disclosed in the forms of ointments, creams, and gels. Suitable ointment excipients include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream excipients may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions disclosed herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable excipients utilized in rectal and vaginal suppositories include bases such as stiffening agents, which produce a melting point in the proximity of body temperature, when formulated with the pharmaceutical compositions disclosed herein; and antioxidants as described herein, including bisulfite and sodium metabisulfite. Suitable excipients include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various excipients may be used. Rectal and vaginal suppositories may be prepared by the compressed method or molding. The typical weight of a rectal and vaginal suppository is about 2 to about 3 g.

The pharmaceutical compositions disclosed herein may be administered ophthalmically in the forms of solutions, suspensions, ointments, emulsions, gel-forming solutions, powders for solutions, gels, ocular inserts, and implants.

The pharmaceutical compositions disclosed herein may be administered intranasally. The pharmaceutical compositions may be in the form of an aerosol or solution for delivery using a pressurized container, pump, spray, atomizer, such as an atomizer using electrohydrodynamics to produce a fine mist, or nebulizer, alone or in combination with a suitable propellant, such as dichlorodifluoromethane, trichlorofluoromethane, dichlorotetrafluoroethane, a hydrofluoroalkane such as 1,1,1,2-tetrafluoroethane (HFA 134A) and 1,1,1,2,3,3,3-heptafluoropropane (HFA 227), carbon dioxide, perfluorinated hydrocarbons such as perflubron, and other suitable gases. The pharmaceutical compositions may also be disclosed as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient(s) disclosed herein, a propellant as solvent; and/or a surfactant, such as sorbitan trioleate, oleic acid, or an oligolactic acid.

The pharmaceutical compositions disclosed herein may be micronized to a size suitable for delivery, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in inhaler or insufflator may be formulated to contain a powder mix of the pharmaceutical compositions disclosed herein; a suitable powder base, such as lactose or starch; and a performance modifier, such as 1-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions disclosed herein for inhaled/intranasal administration may further comprise a suitable flavoring agent, such as menthol and levomenthol, or sweeteners, such as saccharin or saccharin sodium.

The pharmaceutical compositions disclosed herein for topical administration may be formulated to be immediate release or modified release, including delayed-, sustained-, pulsed-, controlled-, targeted, and programmed release.

### D. Modified Release Dosage Forms

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. The pharmaceutical compositions in modified release dosage forms can be prepared using a variety of modified release devices and methods known to those skilled in the art, including, but not limited to, matrix-controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

As used herein, immediate release refers to the release of an active ingredient(s) substantially immediately upon administration. In some embodiments, immediate release occurs when there is dissolution of an active ingredient(s) within 1-20 minutes after administration. Dissolution can be of all or less than all (e.g., about 70%, about 75%, about 80%, about 85%, about 90%, about 91%, about 92%, about 93%, about 94%, about 95%, about 96%, about 97%, about 98%, about 99%, about 99.5%, 99.9%, or 99.99%) of the active ingredient(s). In some embodiments, immediate release results in complete or less than complete dissolution within about 1 hour following administration. Dissolution can be in a subject's stomach and/or intestine. In some embodiments, immediate release results in dissolution of an active ingredient(s) within 1-20 minutes after entering the stomach. For example, dissolution of 100% of active ingredient(s) can occur in the prescribed time. In some embodiments, immediate release is through inhalation, such that dissolution occurs in a subject's lungs.

In some embodiments, the pharmaceutical composition has an onset of therapeutic action of 60, 50, 40, 30, 20, 10, 5 minutes or less. In some embodiments, the pharmaceutical composition has an acute effects duration of 120, 110, 100, 90, 80, 70, 60, 50, 40, 30, 20, 10, 5 minutes or less.

In some embodiments, the pharmaceutical composition described herein is a controlled-release composition. In some embodiments, controlled-release results in dissolution of an active ingredient(s) within 20-180 minutes after entering the stomach. In some embodiments, controlled-release occurs when there is dissolution of an active ingredient(s) within 20-180 minutes after being swallowed. In some embodiments, controlled-release occurs when there is dissolution of an active ingredient(s) within 20-180 minutes after entering the intestine. In some embodiments, controlled-release results in substantially complete dissolution 1 hour or longer following administration, for example the release period can be greater than about 4 hours, 8 hours, 12 hours, 16 hours, or 20 hours. In some embodiments, controlled-release results in substantially complete dissolution 1 hour or longer following oral administration.

### 1. Matrix-Controlled Release Devices

The pharmaceutical compositions disclosed herein in a modified release dosage form may be fabricated using a matrix-controlled release device known to those skilled in the art (see, Takada et al in “Encyclopedia of Controlled Drug Delivery,” Vol. 2, Mathiowitz ed., Wiley, 1999).

In some embodiments, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In some embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate, and fatty compounds, such as carnauba wax, microcrystalline wax, and triglycerides.

In a matrix-controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions disclosed herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions disclosed herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents are water-swellable hydrophilic polymers, which are also referred to as “osmopolymers” and “hydrogels,” include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient(s) metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dime yl inoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

The semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Ve a et al., Drug Development and Industrial Pharmacy 2000, 26, 695-708; Ve a et al., J. Controlled Release 2002, 79, 7-27).

In some embodiments, the pharmaceutical compositions disclosed herein are formulated as AMT controlled-release dosage form, which comprises an asymmetric osmotic membrane that coats a core comprising the active ingredient(s) and other pharmaceutically acceptable excipients or carriers. The AMT controlled-release dosage forms can be prepared according to conventional methods and techniques known to those skilled in the art, including direct compression, dry granulation, wet granulation, and a dip-coating method.

In some embodiments, the pharmaceutical compositions disclosed herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions disclosed herein in a modified release dosage form may be fabricated a multiparticulate controlled release device, which comprises a multiplicity of particles, granules, or pellets, ranging from about 10 m to about 3 mm, about 50 m to about 2.5 mm, or from about 100 m to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions disclosed herein may also be formulated to be targeted to a particular tissue, receptor, or other area of the body of the subject to be treated, including liposome-, resealed erythrocyte-, and antibody-based delivery systems.

### E. Inhalation Administration

The pharmaceutical compositions disclosed herein may be formulated for inhalation administration, e.g., for pulmonary absorption. Suitable preparations may include liquid form preparations such as those described above, e.g., solutions and emulsions, wherein the solvent or carrier is, for example, water, water/water-miscible vehicles such as water/propylene glycol solutions, or organic solvents, with optional buffering agents, which can be delivered as an aerosol, preferably a mist, with or without a carrier gas, such as air, oxygen, a mixture of helium and oxygen, or other gases and gas mixtures including therapeutic gas mixtures. The pharmaceutical compositions may also be formulated as a dry powder for insufflation, alone or in combination with an inert carrier such as lactose or phospholipids.

The pharmaceutical compositions may be in the form of an aerosol or solution for delivery using a pressurized container, pump, spray, atomizer, such as an atomizer using electrohydrodynamics to produce a fine mist, or nebulizer, alone or in combination with a suitable propellant, such as dichlorodifluoromethane, trichlorofluoromethane, dichlorotetrafluoroethane, a hydrofluoroalkane such as 1,1,1,2-tetrafluoroethane (HFA 134A) and 1,1,1,2,3,3,3-heptafluoropropane (HFA 227), carbon dioxide, perfluorinated hydrocarbons such as perflubron, and other suitable gases. Such propellants may be used alone or in addition to nitrous oxide (which when used may serve a dual role as active ingredient and propellant/driving gas). A weight ratio of the 5-HT_{2A} receptor agonist to the propellant present in the aerosol typically ranges from 0.01:100 to 0.1:100, from 0.025:75 to 0.1:75, or for example, 0.05:75, although other ratios may also be used.

Aqueous solutions suitable for inhalation use can be prepared by dissolving the active ingredient(s) in water optionally with other aqueous compatible excipients/co-solvents. Suitable stabilizers and thickening agents can also be added. Emulsions suitable for inhalation use can be made by solubilizing the active ingredient(s) in an aqueous medium and dispersing the solubilized form in a hydrophobic medium, optionally with viscous material, such as natural or synthetic gums, resins, methylcellulose, sodium carboxymethylcellulose, and other suspending agents.

Solutions or suspensions for use in a pressurized container, pump, spray, atomizer, or nebulizer may be formulated to contain a surfactant or other appropriate co-solvent, or a suitable alternative agent for dispersing, solubilizing, or extending release of the active ingredient(s) disclosed herein, and optionally a propellant. Such surfactants or co-solvents may include, but are not limited to, Polysorbate 20, 60, and 80; Pluronic F-68, F-84, and P-103; cyclodextrin; polyoxyl 35 castor oil; sorbitan trioleate, oleic acid, or an oligolactic acid. Surfactants and co-solvents are typically employed at a level between about 0.01% and about 2% by weight of the pharmaceutical composition. Viscosity greater than that of simple aqueous solutions may be desirable in some cases to decrease variability in dispensing the formulations, to decrease physical separation of components of an emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents, when desirable, are typically employed at a level between about 0.01% and about 2% by weight of the pharmaceutical composition.

The active ingredient(s) can also be dissolved in organic solvents or aqueous mixtures of organic solvents. Organic solvents can be, for example, acetonitrile, chlorobenzene, chloroform, cyclohexane, 1,2-dichloromethane, dichloromethane, 1,2-dimethoxyethane, N,N-dimethylacetamide, N,N-dimethylformamide, 1,4-dioxane, 2-ethoxyethanol, ethylene glycol, formamide, hexane, methanol, ethanol, 2-methoxyethanol, methybutylketone, methylcyclohexane, N-methylpyrrolidone, nitromethane, pyridine, sulfolane, tetralin, toluene, 1,1,2-trichloroethylene, or xylene, and like, including combinations thereof. Organic solvents can belong to functional group categories such as ester solvents, ketone solvents, alcohol solvents, amide solvents, ether solvents, hydrocarbon solvents, etc. each of which can be used.

The pharmaceutical composition may also be formulated as a dry powder for inhalation administration, for example, via a dry powder inhalator (DPI). Here, the active ingredient(s) itself can form the powder or the powder can be formed from a pharmaceutically acceptable excipient or carrier and the active ingredient(s) is releasably bound to a surface of the carrier powder such that upon inhalation, the moisture in the lungs releases the active ingredient(s) from the surface to make the drug available for systemic absorption. Examples of carrier particles include, but are not limited to, those made of lactose or other sugars, with mention being made to α-lactose monohydrate.

Further description is provided below relating to pharmaceutical compositions adapted for inhalation and methods for inhalation administration.

### Therapeutic Applications and Methods

The present disclosure is also directed to combination drug therapies and methods for treating a subject with a disease or disorder comprising administering to the subject a therapeutically effective amount of a 5-HT_{2A} receptor agonist and an NMDA receptor antagonist. The disease or disorder may be associated with a 5-HT_{2A} receptor, an NMDA receptor, or both, e.g., a neuropsychiatric disease or disorder, a central nervous system (CNS) disorder and/or a psychological disorder. The combination drug therapy may show enhanced activity and improved patient experience when treating such diseases or disorders, for example, by providing therapeutic efficacy with a slight euphoria, thereby reducing or eliminating psychiatric adverse effects such as acute psychedelic crisis (bad trip) as well as dissociative effects from hallucinogens (out of body experience).

The subjects treated herein may have a disease or disorder associated with a serotonin 5-HT₂ receptor (e.g., 5-HT_{2A} receptor) and/or an NMDA receptor.

In some embodiments, the disease or disorder is a neuropsychiatric disease or disorder.

In some embodiments, the disease or disorder is an inflammatory disease or disorder.

In some embodiments, the disease or disorder is a central nervous system (CNS) disorder and/or a psychiatric disease/psychological disorder, including, but not limited to, post-traumatic stress disorder (PTSD), major depressive disorder (MDD), treatment-resistant depression (TRD), suicidal ideation, suicidal behavior, major depressive disorder with suicidal ideation or suicidal behavior, melancholic depression, atypical depression, dysthymia, non-suicidal self-injury disorder (NSSID), bipolar and related disorders (including, but not limited to, bipolar I disorder, bipolar II disorder, cyclothymic disorder), obsessive-compulsive disorder (OCD), compulsive behavior and other related symptoms, generalized anxiety disorder (GAD), acute psychedelic crisis, social anxiety disorder, substance use disorders (including, but not limited to, alcohol use disorder, opioid use disorder, amphetamine use disorder, nicotine use disorder, cocaine use disorder, and other addictive disorders), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, chronic fatigue syndrome, Lyme disease, gambling disorder, eating disorders (including, but not limited to, anorexia nervosa, bulimia nervosa, binge-eating disorder, etc.), paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido), peripheral neuropathy, and obesity.

In some embodiments, the disease or disorder is major depressive disorder (MDD).

In some embodiments, the disease or disorder is treatment-resistant depression (TRD). TRD is defined herein as MDD with inadequate response to at least two different conventional antidepressants.

In some embodiments, the disease or disorder is anxiety, e.g., generalized anxiety disorder (GAD).

In some embodiments, the disease or disorder is social anxiety disorder.

In some embodiments, the disease or disorder is obsessive-compulsive disorder (OCD).

In some embodiments, the disease or disorder is cancer related depression and anxiety.

In some embodiments, the disease or disorder is headaches (e.g., cluster headache, migraine, etc.).

In some embodiments, the disease or disorder is alcohol use disorder. In some embodiments, the disease or disorder is opioid use disorder. In some embodiments, the disease or disorder is amphetamine use disorder. In some embodiments, the disease or disorder is cocaine use disorder. In some embodiments, the disease or disorder is nicotine use (e.g., smoking) disorder and the therapy is used for smoking cessation.

In some embodiments, the disease or disorder is depression. Types of depression that may be treated with the combination drug therapy of the present disclosure include, but are not limited to, major depression disorder (MDD), melancholic depression, atypical depression, and dysthymia.

In some embodiments, the disease or disorder may include conditions of the autonomic nervous system (ANS).

In some embodiments, the disease or disorder may include pulmonary disorders (e.g., asthma and chronic obstructive pulmonary disorder (COPD).

In some embodiments, the disease or disorder may include cardiovascular disorders (e.g., atherosclerosis).

In some embodiments, the disclosure provides for the management of different kinds of pain, including but not limited to cancer pain, e.g., refractory cancer pain; neuropathic pain; postoperative pain; opioid-induced hyperalgesia and opioid-related tolerance; neurologic pain; postoperative/post-surgical pain; complex regional pain syndrome (CRPS); shock; limb amputation; severe chemical or thermal burn injury; sprains, ligament tears, fractures, wounds and other tissue injuries; dental surgery, procedures and maladies; labor and delivery; during physical therapy; radiation poisoning; acquired immunodeficiency syndrome (AIDS); epidural (or peridural) fibrosis; orthopedic pain; back pain; failed back surgery and failed laminectomy; sciatica; painful sickle cell crisis; arthritis; autoimmune disease; intractable bladder pain; pain associated with certain viruses, e.g., shingles pain or herpes pain; acute nausea, e.g., pain that may be causing the nausea or the abdominal pain that frequently accompanies sever nausea; migraine, e.g., with aura; and other conditions including depression (e.g., acute depression or chronic depression), depression along with pain, alcohol dependence, acute agitation, refractory asthma, acute asthma (e.g., unrelated pain conditions can induce asthma), epilepsy, acute brain injury and stroke, Alzheimer's disease and other disorders. The pain may be persistent or chronic pain that lasts for weeks to years, in some cases even though the injury or illness that caused the pain has healed or gone away, and in some cases despite previous medication and/or treatment. In addition, the disclosure includes the treatment/management of any combination of these types of pain or conditions.

In some embodiments, the pain treated/managed is acute breakthrough pain or pain related to wind-up that can occur in a chronic pain condition. In some embodiments, the pain treated/managed is cancer pain, e.g., refractory cancer pain. In some embodiments, the pain treated/managed is post-surgical pain. In some embodiments, the pain treated/managed is orthopedic pain. In some embodiments, the pain treated/managed is back pain. In some embodiments, the pain treated/managed is neuropathic pain. In some embodiments, the pain treated/managed is dental pain. In some embodiments, the condition treated/managed is depression. In some embodiments, the pain treated/managed is chronic pain in opioid-tolerant patients.

In some embodiments, the disclosure provides for the management of sexual dysfunction, which may include, but is not limited to, sexual desire disorders, for example, decreased libido; sexual arousal disorders, for example, those causing lack of desire, lack of arousal, pain during intercourse, and orgasm disorders such as anorgasmia; and erectile dysfunction; particularly sexual dysfunction disorders stemming from psychological factors.

In some embodiments, the disease or disorder is associated with an NMDA receptor. Diseases or disorders which can be treated through modulation of N-methyl-D-aspartic acid (NMDA) activity, and thus can be treated with the disclosed methods include, but are not limited to, levodopa-induced dyskinesia; dementia (e.g., Alzheimer's dementia), tinnitus, treatment resistant depression (TRD), major depressive disorder, melancholic depression, atypical depression, dysthymia, neuropathic pain, agitation resulting from or associated with Alzheimer's disease, pseudobulbar effect, autism, Bulbar function, generalized anxiety disorder, Alzheimer's disease, schizophrenia, diabetic neuropathy, acute pain, depression, bipolar depression, suicidality, neuropathic pain, and post-traumatic stress disorder (PTSD).

In some embodiments, the disease or disorder is a psychiatric or mental disorder (e.g., schizophrenia, mood disorder, substance induced psychosis, major depressive disorder (MDD), bipolar disorder, bipolar depression (BDep), post-traumatic stress disorder (PTSD), suicidal ideation, anxiety, obsessive compulsive disorder (OCD), and treatment-resistant depression (TRD)).

In some embodiments, the disease or disorder is a neurological disorder (e.g., Huntington's disease (HD), Alzheimer's disease (AD), or systemic lupus erythematosus (SLE)).

The dosage and frequency (single or multiple doses) of the 5-HT_{2A} receptor agonist and the NMDA receptor antagonist can vary depending upon a variety of factors, including, but not limited to, the type and activity of the active ingredient(s) to be administered; the disease/condition being treated; route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans may be determined from animal models. For example, a dose for humans can be formulated to achieve a concentration that has been found to be effective in animals. The dosage in humans can be adjusted by monitoring response to the treatment and adjusting the dosage upwards or downwards.

Dosages may be varied depending upon the requirements of the subject and the active ingredient(s) being employed. The dose administered to a subject, in the context of the pharmaceutical compositions presented herein, should be sufficient to affect a beneficial therapeutic response in the subject over time. The size of the dose also will be determined by the existence, nature, and extent of any adverse side effects. Generally, treatment is initiated with smaller dosages, which are less than the optimum dose. Thereafter, the dosage is increased by small increments until the optimum effect under circumstances is reached.

Dosage amounts and intervals can be adjusted individually to provide levels of the administered active ingredients effective for the particular clinical indication being treated. This will provide a therapeutic regimen that is commensurate with the severity of the individual's disease state.

Administration of the combination drug therapy may be systemic or local. In some embodiments, administration to a mammal will result in systemic release of the 5-HT_{2A} receptor agonist, the NMDA receptor antagonist, or both (for example, into the bloodstream). Routes of administration may include oral routes (e.g., enteral/gastric delivery, intraoral administration such buccal, lingual, and sublingual routes), parenteral routes (e.g., intravenous, intraarterial, intraperitoneal, intrathecal, intraventricular, intraurethral, intrasternal, intracranial, intramuscular, intrasynovial, and subcutaneous administration), topical routes (e.g., (intra)dermal, conjuctival, intracorneal, intraocular, ophthalmic, auricular, transdermal, nasal, vaginal, uretheral, respiratory, and rectal administration), and inhalation routes, or other routes sufficient to affect a beneficial therapeutic response.

The combination drug therapy is intended to embrace administration of the 5-HT_{2A} receptor agonist and the NMDA receptor antagonist (e.g., nitrous oxide, ketamine, etc.) in a sequential manner, that is, wherein each active ingredient is administered at a different time, as well as administration of these active ingredients, or at least two of the active ingredients, in a concurrent manner. Concurrent administration can be accomplished, for example, by administering to the subject a single dosage form having a fixed ratio of each active ingredient or in multiple, single dosage forms for each of the active ingredients. Whether through sequential administration or concurrent administration with separate pharmaceutical compositions, the active ingredients can be administered by the same route or by different routes. The combination drug therapy may involve administration of the NMDA receptor antagonist (e.g., nitrous oxide) at a time preceding the administration of the 5-HT_{2A} receptor agonist, with the 5-HT_{2A} receptor agonist, during the period of therapeutic relevance of the 5-HT_{2A} receptor agonist, during the period immediately after the therapeutically relevant period of the 5-HT_{2A} receptor agonist, or any combination thereof. As a non-limiting example, the NMDA receptor antagonist (e.g., nitrous oxide) may be administered prior to administration commencement of the 5-HT_{2A} receptor agonist and may continue throughout the 5-HT_{2A} receptor agonist administration duration. In some embodiments, the 5-HT_{2A} receptor agonist and the NMDA receptor antagonist are administered sequentially. In some embodiments, the 5-HT_{2A} receptor agonist and the NMDA receptor antagonist are administered concurrently but separately (e.g., separate compositions, dosage forms, or routes of administration). In some embodiments, the 5-HT_{2A} receptor agonist and the NMDA receptor antagonist are administered concurrently in the same dosage form.

In some embodiments, the 5-HT_{2A} receptor agonist and the NMDA receptor antagonist are each administered via inhalation, in the same dosage form or separate dosage forms. In preferred embodiments, the NMDA receptor antagonist is nitrous oxide, which is concurrently administered with the 5-HT_{2A} receptor agonist in aerosolized form. For example, nitrous oxide may be administered concurrently (e.g., simultaneously) with the 5-HT_{2A} receptor agonist via an aerosol, whereby nitrous oxide may dually act as a propellant or carrier gas for the aerosol generation and as an active ingredient of the aerosol composition. The inhalation administration may be performed on a continual basis, for example, over any desired duration, e.g., 5 minutes, 10 minutes 15 minutes, 20 minutes, 30 minutes, 40 minutes, 45 minutes, 50 minutes, 60 minutes, 90 minutes, 120 minutes, 150 minutes, 180 minutes, or any range therebetween. In some embodiments, the 5-HT_{2A} receptor agonist and the NMDA receptor antagonist are each administered via inhalation, in separate dosage forms. In some embodiments, the NMDA receptor antagonist is nitrous oxide, which is administered as a therapeutic gas mixture, and the 5-HT_{2A} receptor agonist is administered as an aerosol, preferably a mist.

In some embodiments, the 5-HT_{2A} receptor agonist and the NMDA receptor antagonist (e.g., ketamine) are each administered transdermally or subcutaneously, preferably from the same dosage form, e.g., the same transdermal patch.

In some embodiments, the 5-HT_{2A} receptor agonist is administered via parenteral injection (e.g., intravenous) and the NMDA receptor antagonist (e.g., nitrous oxide) is administered via inhalation, such as in a therapeutic gas mixture. When administered parenterally, the 5-HT_{2A} receptor agonist may be given in bolus form, as a perfusion, or as both a bolus and perfusion.

In some embodiments, the 5-HT_{2A} receptor agonist is administered orally while the NMDA receptor antagonist (e.g., nitrous oxide) is administered via inhalation, such as in a therapeutic gas mixture.

In some embodiments, all active ingredients are administered orally or intranasally.

When the 5-HT_{2A} receptor agonist and the NMDA receptor antagonist are administered concurrently, 5-HT_{2A} receptor agonist and the NMDA receptor antagonist (e.g., nitrous oxide) may be administered at the same time (e.g., when administered within the same dosage form, such as within the same aerosol or within the same transdermal patch), at overlapping times (e.g., where the 5-HT_{2A} receptor agonist is administered at some point during administration of the NMDA receptor antagonist such as during an inhalation session with nitrous oxide), or at non-overlapping times but separated by no more than 30 seconds, i.e., where the start of administration of a first active ingredient (e.g., the 5-HT_{2A} receptor agonist) is separated from the end time of administration of a second active ingredient (e.g., the NMDA receptor antagonist), or vice versa, by no more than 30 seconds. The interval between non-overlapping administration may be no more than 30 seconds, no more than 20 seconds, no more than 15 seconds, no more than 10 seconds, no more than 5 seconds, no more than 4 seconds, no more than 3 seconds, no more than 2 seconds, no more than 1 second.

When the 5-HT_{2A} receptor agonist and the NMDA receptor antagonist (e.g., nitrous oxide) are administered sequentially (i.e., separately), the interval of time between their non-overlapping administration, i.e., their administration start/end points, may range from greater than 30 seconds, 1 minute, 2 minutes, 3 minutes, 4 minutes, 5 minutes, 10 minutes, 20 minutes, 30 minutes, 40 minutes, 50 minutes, 1 hour, 2 hours, 3 hours, 4 hours, 5 hours, 8 hours, 10 hours, 12 hours, 18 hours, 1 day, 2 days, 3 days, 4 days, 5 days, 6 days, 1 week, or longer (e.g., 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months, 5 months, 6 months, 7 months, 8 months, 9 months, 10 months, 11 months, 12 months, 1 year, 2 years) or any period of time in between. For sequential administration, the 5-HT_{2A} agonist and the NMDA receptor antagonist are preferably administered from greater than 30 seconds to less than 1 minute, less than 2 minutes, less than 2 minutes, less than 3 minutes, less than 4 minutes, less than 5 minutes, less than 10 minutes, less than 15 minutes, less than 30 minutes, less than 45 minutes, less than 1 hour, less than 2 hours, or less than 4 hours apart.

Administration may follow a continuous administration schedule, or an intermittent administration schedule. The administration schedule may be varied depending on the active ingredients employed, the condition being treated, the administration route, etc. For example, administration of one or both of the 5-HT_{2A} receptor agonist and the NMDA receptor antagonist may be performed once a day (QD), or in divided dosages throughout the day, such as 2-times a day (BID), 3-times a day (TID), 4-times a day (QID), or more. In some embodiments administration may be performed nightly (QHS). In some embodiments, administration is performed as needed (PRN). Administration may also be performed on a weekly basis, e.g., once a week, twice a week, three times a week, four times a week, every other week, every two weeks, etc. The administration schedule may also designate a defined number of treatments per treatment course, for example, the 5-HT_{2A} receptor agonist and the NMDA receptor antagonist may be co-administered, together or separately, 1 time, 2 times, 3 times, 4 times, 5 times, 6 times, 7 times, or 8 times per treatment course. Other administration schedules may also be deemed appropriate using sound medical judgement

The dosing can be continuous (7 days of administration in a week) or intermittent, for example, depending on the pharmacokinetics and a particular subject's clearance/accumulation of the active ingredient(s). If intermittently, the schedule may be, for example, 4 days of administration and 3 days off (rest days) in a week or any other intermittent dosing schedule deemed appropriate using sound medical judgement. The dosing whether continuous or intermittent is continued for a particular treatment course, typically at least a 28-day cycle (1 month), which can be repeated with or without a drug holiday. Longer or shorter courses can also be used such as 14 days, 18 days, 21 days, 24 days, 35 days, 42 days, 48 days, or longer, or any range therebetween. The course may be repeated without a drug holiday or with a drug holiday depending upon the subject. Other schedules are possible depending upon the presence or absence of adverse events, response to the treatment, patient convenience, and the like.

In some embodiments, the combination drug therapy of the present disclosure may be used as a standalone therapy. In some embodiments, the combination drug therapy may be used as an adjuvant/combination therapy with other treatment modalities and/or agents. For example, treatment with the 5-HT_{2A} receptor agonist and the NMDA receptor antagonist may be performed in conjunction with psychotherapy, psycho-social therapy (e.g., cognitive behavioral therapy), and/or treatment with other agents such as an anxiolytic or antidepressant (conventional). Examples of anxiolytics/antidepressants include, but are not limited to, barbiturates; benzodiazepines such as alprazolam, bromazepam, chlordiazepoxide, clonazepam, diazepam, lorazepam, oxazepam, temazepam, and triazolam; selective serotonin reuptake inhibitors (SSRIs) such as citalopram, escitalopram, fluoxetine, fluvoxamine, paroxetine, and sertraline; serotonin-norepinephrine reuptake inhibitors (SNRIs) such as venlafaxine, duloxetine, atomoxetine, desvenlafaxine, levomilnacipran, milnacipran, sibutramine, and tramadol; serotonin modulator and stimulators (SMSs) such as vortioxetine and vilazodone; serotonin antagonist and reuptake inhibitors (SARIs) such as trazodone and nefazodone; norepinephrine reuptake inhibitors (NRIs or NERIs) such as atomoxetine, reboxetine, and viloxazine; norepinephrine-dopamine reuptake inhibitors such as bupropion; tricyclic antidepressants (TCAs) such as imipramine, doxepin, amitriptyline, nortriptyline and desipramine; tetracyclic antidepressants such as mirtazapine; monoamine oxidase inhibitors (MAOIs) such as phenelzine, isocarboxazid, tranylcypromine and pyrazidol; sympatholytics such as propranolol, oxprenolol, metoprolol, prazosin, clonidine, and guanfacine; and others such as buspirone, pregabalin, and hydroxyzine.

The administering physician can provide a method of treatment that is prophylactic or therapeutic by adjusting the amount and timing of any of the active ingredients described herein on the basis of observations of one or more symptoms of the disorder or condition being treated. Utilizing the teachings provided herein, an effective prophylactic or therapeutic treatment regimen can be planned that does not cause substantial toxicity or adverse side effects (e.g., caused by sedative or psychotomimetic toxic spikes in plasma concentration of any of the active ingredient(s)), and yet is entirely effective to treat the clinical symptoms demonstrated by the particular patient. This planning should involve the careful choice of active ingredients by considering factors such as compound potency, relative bioavailability, patient body weight, presence and severity of adverse side effects, preferred mode of administration, and the toxicity profile of the selected active ingredients. In some embodiments, the subject is a mammal. In some embodiments, the mammal is a human.

A therapeutically or prophylactically effective dose herein may vary depending on the variety of factors described above, but is typically that which provides the 5-HT_{2A} receptor agonist and/or the NMDA receptor antagonist in an amount of about 0.00001 mg to about 10 mg per kilogram body weight of the recipient, or any range in between, e.g., about 0.00001 mg/kg, about 0.00005 mg/kg, about 0.0001 mg/kg, about 0.0005 mg/kg, about 0.001 mg/kg, about 0.005 mg/kg, about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg.

In some embodiments, the 5-HT_{2A} receptor agonist may be administered at a psychedelic dose, for example, at a dose of from greater than about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, and up to about 1 mg/kg, about 0.95 mg/kg, about 0.9 mg/kg, about 0.85 mg/kg, about 0.8 mg/kg, about 0.75 mg/kg, about 0.7 mg/kg, about 0.65 mg/kg, about 0.6 mg/kg, about 0.55 mg/kg, in conjunction with an appropriate dosage of the NMDA receptor antagonist.

In some embodiments, the 5-HT_{2A} receptor agonist (e.g., DMT, DMT-d₁₀, etc.) is administered to the subject intravenously as a single bolus per treatment session within the dosage range described above, e.g., about 0.1 mg/kg to about 0.8 mg/kg, or about 0.2 mg/kg to about 0.5 mg/kg, or about 0.3 mg/kg. In some embodiments, the 5-HT_{2A} receptor agonist (e.g., DMT, DMT-d₁₀, etc.) is administered to the subject as a perfusion during a treatment session within the dosage range described above, e.g., about 0.1 mg/kg to about 0.8 mg/kg, or about 0.2 mg/kg to about 0.5 mg/kg, or about 0.45 mg/kg. The perfusion may be administered over a duration of about 5 minutes, about 10 minutes, about 20 minutes, about 30 minutes, about 40 minutes, about 50 minutes, about 1 hour, about 2 hours, about 3 hours, about 4 hours, about 5 hours, for example. The 5-HT_{2A} receptor agonist may be administered via perfusion at a rate of about 0.1 mg/min, 0.2 mg/min, 0.3 mg/min, 0.4 mg/min, 0.5 mg/min, 0.6 mg/min, 0.7 mg/min, 0.8 mg/min, 0.9 mg/min, 1 mg/min, 1.5 mg/min, 2 mg/min, 2.5 mg/min, 3 mg/min, 3.5 mg/min, 4 mg/min, 4.5 mg/min, 5 mg/min, or otherwise as deemed appropriate by a medical professional. In some embodiments, the 5-HT_{2A} receptor agonist (e.g., DMT, DMT-d₁₀, etc.) is administered to the subject intravenously as a bolus within the dosage range described above, e.g., about 0.1 mg/kg to about 0.8 mg/kg, or about 0.2 mg/kg to about 0.5 mg/kg, or about 0.3 mg/kg, followed by a perfusion within the dosage range described above, e.g., about 0.1 mg/kg to about 0.8 mg/kg, or about 0.2 mg/kg to about 0.5 mg/kg, or about 0.45 mg/kg. The NMDA receptor antagonist may be administered concurrently or sequentially with administration of the 5-HT_{2A} receptor agonist, for example through inhalation of a therapeutic gas mixture containing nitrous oxide. When the combination drug therapy is not administered simultaneously (not in unison), it is preferred that administration of the NMDA receptor antagonist is commenced prior to commencement of administration of the 5-HT_{2A} receptor agonist.

The aforementioned psychedelic doses are typically administered 1 time, 2 times, 3 times, 4 times, 5 times, 6 times, 7 times, or 8 times in any one course of treatment. Courses can be repeated as necessary, with or without a drug holiday. Such treatment regimens may be accompanied by psychotherapy, before, during, and/or after the psychedelic dose. These treatments may be appropriate for a variety of mental health disorders disclosed herein, examples of which include, but are not limited to, major depressive disorder (MDD), therapy resistant depression (TRD), anxiety disorders, and substance use disorders (e.g., alcohol use disorder, opioid use disorder, amphetamine use disorder, nicotine use disorder, smoking, and cocaine use disorder).

The 5-HT_{2A} receptor agonist and/or the NMDA receptor antagonist may be administered at serotonergic, but sub-psychedelic concentrations to achieve durable therapeutic benefits, with decreased toxicity, and may thus be suitable for microdosing. The dose range for sub-psychedelic dosing may range from about 0.00001 mg/kg, about 0.00005 mg/kg, about 0.0001 mg/kg, about 0.0005 mg/kg, about 0.001 mg/kg, about 0.005 mg/kg, about 0.006 mg/kg, about 0.008 mg/kg, about 0.009 mg/kg, about 0.01 mg/kg, and up to about 0.1 mg/kg, about 0.09 mg/kg, about 0.083 mg/kg, about 0.08 mg/kg, about 0.075 mg/kg, about 0.07 mg/kg, about 0.06 mg/kg, about 0.05 mg/kg, about 0.04 mg/kg, about 0.03 mg/kg, about 0.02 mg/kg of the active ingredient(s). Typically, sub-psychedelic doses are administered every day, for a treatment course (e.g., 1 month). However, there is no limitation on the number of doses at sub-psychedelic doses-dosing can be less frequent or more frequent as deemed appropriate. Courses can be repeated as necessary, with or without a drug holiday.

Sub-psychedelic dosing can also be carried out, for example, by transdermal delivery, subcutaneous administration, etc., via modified, controlled, slow, or extended release dosage forms, including, but not limited to, depot dosage forms, implants, patches, and pumps, which can be optionally remotely controlled. Here, doses would be adapted to provide sub-psychedelic blood levels of one or both of the 5-HT_{2A} receptor agonist and the NMDA receptor antagonist. In some embodiments, the 5-HT_{2A} receptor agonist (e.g., DMT, DMT-d₁₀, etc.) and the NMDA receptor antagonist (e.g., (S)-ketamine) are administered transdermally via a patch, such as a drug-in-adhesive (DIA) transdermal patch.

The selection of a 5-HT_{2A} receptor agonist containing deuteration (e.g., DMT-d₁₀, 5-MeO-DMT-d₁₀, etc.) may be particularly advantageous for sub-psychedelic dosing, as these compounds possess desirable metabolic degradation profiles which prevent high drug concentrations observed acutely after administration, while also enhancing brain levels of the active compound, which enables the therapeutic doses to be reduced. Accordingly, these 5-HT_{2A} receptor agonists may be administered chronically at serotonergic, but sub-psychoactive concentrations with decreased toxicity, e.g., toxicity associated with activation of 5-HT_{2B} receptors associated with valvular heart disease (Rothman, R. B., and Baumann, M. H., 2009, Serotonergic drugs and valvular heart disease, Expert Opin Drug Saf 8, 317-329).

Sub-psychedelic doses can be used, e.g., for the chronic treatment a variety of diseases or disorders disclosed herein, examples of which include, but are not limited to, inflammation, pain and neuroinflammation.

In some embodiments, the co-administration of the 5-HT_{2A} receptor agonist and the NMDA receptor antagonist (e.g., nitrous oxide in the form of a therapeutic gas mixture comprising nitrous oxide in concentrations disclosed herein) can reduce the effective amount of 5-HT_{2A} receptor agonist to be delivered by about 2, 5, 10, 20, 30, 40, 50, 60, 70 percent or more, as compared to a dose not delivered with the NMDA receptor antagonist as described herein. The lower amount of the 5-HT_{2A} receptor agonist can result in fewer or less severe side effects such as psychological disorders such as acute psychedelic crisis (a bad trip), dysphoric physiological and psychological side effects, nausea, headache, anxiety, emotional discomfort, confusion, dizziness, and sedation. For example, the amount and/or severity of nausea, headache, anxiety, emotional discomfort, confusion, dizziness, and sedation can be reduced when low levels of nitrous oxide (e.g., a level of about 5-25%) is used.

Efficacy of the combination drug therapy may in some cases be assessed through clinical interviews where patients answer a series of questionnaires, which allows for quantification of different aspects of psychedelic-induced subjective effects. These assessments can include, but are not limited to, Mystical Experience Questionnaire-30 Item (MEQ-30) (see Maclean, K. A., Leoutsakos, J.-M. S., Johnson, M. W. & Griffiths, R. R. Factor Analysis of the Mystical Experience Questionnaire: A Study of Experiences Occasioned by the Hallucinogen Psilocybin. J Sci Study Relig 51, 721-737 (2012)), 5-Dimensional Altered States of Consciousness Rating Scale (5D-ASC) (see Dittrich, A. The Standardized Psychometric Assessment of Altered States of Consciousness (ASCs) in Humans. Pharmacopsychiatry 31, 80-84 (1998)), and the Hallucinogen Rating Scale (HRS) (see Strassman, R. J., Qualls, C. R., Uhlenhuth, E. H. & Kellner, R. Dose-Response Study of N,N-Dimethyltryptamine in Humans: II. Subjective Effects and Preliminary Results of a New Rating Scale. Archives of General Psychiatry 51, 98-108 (1994)). In some embodiments, the combination drug therapy disclosed herein results in greater scores in the MEQ-30, 5D-ASC and/or HRS assessments compared to scores obtained from either the 5-HT_{2A} receptor agonist or the NMDA receptor antagonist administered alone.

The combination drug therapy of the present disclosure may decrease, inhibit, or eliminate occurrences of psychiatric adverse effects such as acute psychedelic crisis and/or dissociative effects experienced by the patient, compared to when the 5-HT_{2A} receptor agonist or the NMDA receptor antagonist are taken alone. The quantification of negative experiences may in some cases be assessed through assessments including, but not limited to. The Brief Psychiatric Rating Scale (BPRS), the Patient Rating Inventory of Side Effects (PRISE), Challenging Experience Questionnaire (CEQ) (see Barrett, F. S., Bradstreet, M. P., Leoutsakos, J.-M. S., Johnson, M. W. & Griffiths, R. R. The Challenging Experience Questionnaire: Characterization of challenging experiences with psilocybin mushrooms. J Psychopharmacol 30, 1279-1295 (2016)), and The Clinician-Administered Dissociative State Scale (CADSS), with CADSS being used to measure dissociative effects during the treatment. In some embodiments, the combination drug therapy disclosed herein results in lower scores in the CEQ assessment, particularly in ratings of fear and physical distress, compared to scores obtained from administration of the 5-HT_{2A} receptor agonist alone.

In the case wherein the patient's condition does not improve, upon the doctor's discretion the combination drug therapy may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the combination drug therapy may be given continuously or temporarily suspended for a certain length of time (i.e., a drug holiday).

Once improvement of the patient's conditions has occurred, a maintenance dose may be administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

In some embodiments, the NMDA receptor antagonist used in the combination drug therapy is nitrous oxide. Nitrous oxide may be administered alone, or as a therapeutic gas mixture, e.g., N₂ and O₂; N₂O and air; N₂O and medical air (medical air being 78% nitrogen, 21% oxygen, 1% other gases); N₂O and a N₂/O₂ mix; N₂O and O₂ enriched medical air; N₂O and a He/O₂ mix etc. Thus, in addition to nitrous oxide and oxygen, the therapeutic gas mixture may further include other gases such as one or more of N₂, Ar, CO₂, Ne, CH₄, He, Kr, H₂, Xe, H₂O (e.g., vapor), etc. For example, nitrous oxide may be administered using a blending system that combines N₂O, O₂ and optionally other gases from separate compressed gas cylinders into a therapeutic gas mixture which is delivered to a patient via inhalation. Alternatively, the therapeutic gas mixture containing nitrous oxide may be packaged, for example, in a pressurized tank or in small, pressurized canisters which are easy to use and/or portable. The blending system and/or pressurized tanks/canisters may be adapted to fluidly connect to an inhalation device such as a device capable of generating an aerosol of the 5-HT_{2A} receptor agonist. Nitrous oxide itself, or the therapeutic gas mixture comprising nitrous oxide may be used for the generation of the aerosol (i.e., as the gas phase component of the aerosol) or as a carrier gas to facilitate the transfer of a generated aerosol to a patient's lungs. In some embodiments, N₂O is present in the therapeutic gas mixture at a concentration ranging from 5 vol %, from 10 vol %, from 15 vol %, from 20 vol %, from 25 vol %, from 30 vol %, from 35 vol %, from 40 vol %, from 45 vol %, and up to 75 vol %, up to 70 vol %, up to 65 vol %, up to 60 vol %, up to 55 vol %, up to 50 vol %, relative to a total volume of the therapeutic gas mixture.

Previously, mixtures of nitrous oxide and oxygen have been proposed to treat MDD and TRD (see, e.g., Nagele, P. et al. Biol. Psych. 2015 and Nagele, P. et al. Science Transl, Med., 2021), showing efficacy at 50/50 mixtures and 25/75 mixtures of nitrous oxide/oxygen, with 1 hour treatment regimens. The present inventors have found, however, that lower levels of nitrous oxide, for the same time period or less, can provide similar efficacy but with a significantly reduced side effect profile. Thus, in some embodiments, N₂O is administered in a therapeutic gas mixture, concurrently with, or in some instances sequentially with (separately from), the 5-HT_{2A} receptor agonist, at a concentration ranging from 5 vol %, from 10 vol %, from 15 vol %, from 16 vol %, from 17 vol %, from 18 vol %, from 19 vol %, and up to 25 vol %, up to 24 vol %, up to 23 vol %, up to 22 vol %, up to 21 vol %, up to 20 vol %, relative to a total volume of the therapeutic gas mixture. In some embodiments, nitrous oxide is employed in concentrations which does not put the patient to sleep. The therapeutic gas mixture containing nitrous oxide can be administered over any desired duration, e.g., 5 minutes, 10 minutes 15 minutes, 20 minutes, 30 minutes, 40 minutes, 45 minutes, 50 minutes, 60 minutes, 90 minutes, 120 minutes, 150 minutes, 180 minutes, or any range therebetween.

Methods of delivering the combination drug therapy to a patient in need thereof may comprise administering the 5-HT_{2A} receptor agonist and/or the NMDA receptor antagonist in an aerosol, preferably a mist, via inhalation. Delivery of the 5-HT_{2A} receptor agonist may be useful in the treatment of a disease or disorder, such as a disease or disorder associated with a serotonin 5-HT₂ receptor, e.g., inter alia, a central nervous system (CNS) disorder and/or psychological disorder, as described herein. Preferably, the aerosol is generated without externally added heat (this does not exclude minor temperature increases caused by the formation of the aerosol itself, such as with a vibrating mesh or other nebulizer. However, such minor temperature increases can often be offset by vaporization of the drug, which results in cooling of the composition). In some embodiments, the 5-HT_{2A} receptor agonist and/or the NMDA receptor antagonist can be delivered as an aerosol, preferably a mist. The NMDA receptor antagonist (e.g., nitrous oxide) can be present in the gas phase of the aerosol, or in a carrier gas used to deliver a generated aerosol to the patient's lungs. The carrier gas can comprise air, oxygen, a mixture of helium and oxygen, or other gas mixtures including therapeutic gas mixtures. The carrier gas can in some instances be a mixture of helium and oxygen heated to about 50° C. to about 60° C. The aerosol may be generated from a pressurized container, pump, spray, atomizer, or nebulizer, with or without the use of a propellant gas. Preferably, the aerosol composition comprises a solution or suspension of the 5-HT_{2A} receptor agonist, optionally with a propellant gas, which can be atomized into an aerosol (e.g., mist) for inhalation therapy. The aerosol may, or may not, have a gas phase comprising the NMDA receptor antagonist (e.g., nitrous oxide).

Additionally, by administration via inhalation, the 5-HT_{2A} receptor agonist and/or the NMDA receptor antagonist can be delivered systemically to the patient's central nervous system. The carrier gas, e.g., air, oxygen, a mixture of helium and oxygen, medical air, a N₂/O₂ gas mix, O₂ enriched medical air, or other gases and gas mixtures, can be heated to about 50° C. to about 60° C., or to about 55° C. to about 56° C. When a mixture of helium and oxygen is used as the carrier, the helium can be present in the mixture of oxygen and helium at about 50%, 60%, 70%, 80% or 90% by volume, and the oxygen can be present in the mixture at about 50%, 40%, 30%, or 10% by volume, or any range therebetween.

The method can further comprise administering a pretreatment inhalation therapy prior to administration of the aerosol comprising the 5-HT_{2A} receptor agonist and/or the NMDA receptor antagonist. The pretreatment can comprise administering via inhalation of a mixture of helium and oxygen heated to about 90° C., to about 92° C., to about 94° C., to about 96° C., to about 98° C., to about 100° C., to about 105° C., to about 110° C., to about 115° C., to about 120° C., or any range therebetween, to the patient.

The method can comprise (i) administering via inhalation a mixture of helium and oxygen heated to about 90° C. to about 120° C. to the patient, followed by (ii) administering via inhalation a mixture of helium and oxygen heated to about 50° C. to about 60° C. and the aerosol comprising the 5-HT_{2A} receptor agonist and/or the NMDA receptor antagonist to the patient and then repeating steps (i) and (ii). Steps (i) and (ii) can be repeated 1, 2, 3, 4, 5, or more times.

In some embodiments, the present disclosure provides a method of treating a central nervous system (CNS) disorder and/or psychological disorder comprising administering, via inhalation, the 5-HT_{2A} receptor agonist and/or the NMDA receptor antagonist in the form of an aerosol, preferably a mist. The 5-HT_{2A} receptor agonist can be delivered as an aerosol along with a carrier gas e.g., air, oxygen, a mixture of helium and oxygen, or other gases and gas mixtures including therapeutic gas mixtures comprising nitrous oxide. The mixture of helium and oxygen c be heated to about 50° C. to about 60° C. prior to administering the aerosol comprising the 5-HT_{2A} receptor agonist to the patient.

The central nervous system and/or psychological disorder can be, for example, any of those disclosed herein, with specific mention being made to a substance use disorder (e.g., alcohol use disorder), generalized anxiety disorder (GAD), social anxiety disorder, and treatment-resistant depression (TRD).

In some embodiments, the 5-HT_{2A} receptor agonist is delivered by inhalation to the patient's central nervous system resulting in an improvement in drug bioavailability by at least 25% as compared to oral delivery, increased Cₘₐₓ by at least 25% as compared to oral delivery, reduced Tₘₐₓ by at least 50% as compared to oral delivery, or a combination thereof.

The combination drug therapy can be administered via inhalation, preferably as a mist, at about 1 μg to about 100 mg or more (or any range between about 1 μg to about 100 mg) of each active ingredient, e.g., about 1 μg, 2 μg, 5 μg, 6 μg, 10 μg, 13 μg, 15 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 400 μg, 500 μg, 1.0 mg, 2.0 mg, 3.0 mg, 4.0 mg, 5.0 mg, 6.0 mg, 7.0 mg, 8.0 mg, 9.0 mg, 10.0 mg, 20.0 mg, 30.0 mg, 40.0 mg, 50.0 mg, 60.0 mg, 70.0 mg, 80.0 mg, 90.0 mg, 100.0 mg, or more of each of the active ingredient, per inhalation session. In some embodiments, a subject can have 1, 2, 3, 4, 5 or more inhalation sessions a day. In some embodiments, a subject can have 1, 2, 3, 4, 5 or more inhalation sessions every other day, twice a week, or three times a week. In some embodiments, a subject can have 1, 2, 3, 4, 5 or more inhalation sessions every other month, twice a month, three times a month, or four times a month. In some embodiments, a subject can have 1, 2, 3, 4, 5, 6, 7, 8, or more inhalation sessions per treatment course, such as within a 28-day time period.

### Aerosols

In some embodiments, methods of delivering the 5-HT_{2A} receptor agonist and/or the NMDA receptor antagonist by aerosol inhalation are provided. An aerosol, preferably a mist, can be formed from, as the gas phase, air, oxygen, a mixture of helium and oxygen, medical air, a N₂/O₂ gas mix, O₂ enriched medical air, or other gases and gas mixtures including therapeutic gas mixtures. A carrier gas can also be used to facilitate delivery of the aerosol to the patient's lungs. The carrier gas can be delivered at room temperature or heated. In some embodiments, an aerosol, preferably a mist comprising the 5-HT_{2A} receptor agonist is delivered via inhalation using heated helium-oxygen (HELIOX) mixtures. Due to very low viscosity of helium the helium-oxygen mixtures generate gaseous streams characterized by laminar flow that is a highly desirable feature for reaching out into the deep lung areas and reducing deposition of the drug in the respiratory tract, one of the major obstacles in dose delivery via inhalation. A patient can inhale the 5-HT_{2A} receptor agonist and/or the NMDA receptor antagonist disclosed herein as a mist into an alveolar region of the patient's lungs. The active ingredient(s) can then be delivered to a fluid lining of the alveolar region of the lungs and can be systemically absorbed into patient blood circulation. Advantageously, these formulations can be effectively delivered to the blood stream upon inhalation to the alveolar regions of the lungs.

Devices suitable for delivery of heated or unheated gas phase or carrier gas (e.g., air, oxygen, or helium-oxygen mixtures) include, for example, continuous mode nebulizers Flo-Mist (Phillips) and Hope (B & B Medical Technologies) and the accessories such as regulators, e.g., Medipure™ Heliox-LCQ System (PraxAir) and control box, e.g., Precision Control Flow (PraxAir). In some embodiments, a full delivery setup can be a device as described in, for example, Russian patent RU199823 UL.

The term “heliox” as used herein refers to breathing gas mixtures of helium gas (He) and oxygen gas (O₂). In some embodiments, the heliox mixture can contain helium in the mixture of helium and oxygen at about 50%, 60%, 70%, 80% or 90% by volume, and contain oxygen in the mixture of helium and oxygen at about 50%, 40%, 30%, or 10% by volume, or any range therebetween. The heliox mixture can thus contain helium and oxygen in a volume ratio of 50:50, 60:40, 70:30, 80:20, 90:10, or any range therebetween. In some embodiments, heliox can generate less airway resistance through increased tendency to laminar flow and reduced resistance in turbulent flow.

The use of heat in heliox mixtures can further enhance drug delivery by increasing permeability of key physical barriers for drug absorption. Heating of mucosal surfaces can increase permeability by enhancing peripheral blood circulation and relaxing the interstitial junction, as well as other mechanisms. Helium has a thermal conductivity almost 10 times higher than oxygen and nitrogen and can facilitate heat transfer more efficiently. A dry heliox mixture can be used safely as a pretreatment step when warmed up to as high as 110° C., which can enable the dry heliox mixture to heat mucosal surfaces of the lung and respiratory tract more efficiently.

Various types of personal vaporizers are known in the art. In general, personal vaporizers are characterized by heating a solid drug or compound. Vaporizers can work by directly heating a solid drug or compound to a smoldering point. Vaporizing a solid or solid concentrate can be done by convection on conduction. Convection heating of solid concentrate involves a heating element coming into contact with water, or another liquid, which then vaporizes. The hot vapor in turn directly heats the solid or solid concentrate to a smoldering point, releasing a vapor that is inhaled by a user. Conduction heating involves direct contact between the solid or solid concentrate and the heating element, which brings the solid to a smoldering point, releasing vapor to be inhaled by a user. Though vaporizers present advantages over smoking in terms of lung damage, the active ingredient(s) that is vaporized can be substantially deteriorated by the vaporizing heat.

In some embodiments, the 5-HT_{2A} receptor agonist is delivered via a nebulizer, which generates an aqueous-droplet aerosol, preferably a mist, containing the 5-HT_{2A} receptor agonist, which is optionally combined with a heated helium-oxygen mixture. In some embodiments, the 5-HT_{2A} receptor agonist is delivered via a nebulizer, which generates an aqueous-droplet aerosol, preferably a mist, containing the 5-HT_{2A} receptor agonist, which is combined with a driving gas comprising nitrous oxide. The driving gas comprising nitrous oxide may be nitrous oxide gas itself or a therapeutic gas mixture, such as N₂O and O₂; N₂O and air; N₂O and medical air; N₂O and a N₂/O₂ mix; N₂O and O₂ enriched medical air; etc. The therapeutic gas mixture may further include other gases such as one or more of N₂, Ar, CO₂, Ne, CH₄, He, Kr, H₂, Xe, H₂O (e.g., vapor), etc. In some embodiments, the driving gas is a therapeutic gas mixture comprising N₂O, which is present at a concentration ranging from 5 vol %, from 10 vol %, from 15 vol %, from 20 vol %, from 25 vol %, from 30 vol %, from 35 vol %, from 40 vol %, from 45 vol %, and up to 75 vol %, up to 70 vol %, up to 65 vol %, up to 60 vol %, up to 55 vol %, up to 50 vol %, relative to a total volume of the therapeutic gas mixture, or any range in between. The presence of nitrous oxide (being an NMDA receptor antagonist) in (or as) the driving gas can augment the effect of the disclosed 5-HT_{2A} receptor agonists and provide the ability to use less of 5-HT_{2A} receptor agonist to obtain similar levels of effect. Thus, in preferred embodiments, the methods of treating a central nervous system (CNS) disorder or a psychiatric disease comprise administering a pharmaceutical composition containing the combination drug therapy as an aerosol (e.g., mist) via inhalation using a nebulizer. The treatment can alleviate one or more symptoms of the disorder or disease.

For example, a preparation of a 5-HT_{2A} receptor agonist can be placed into a liquid medium and put into an aerosol by a device, such as a nebulizer. In some embodiments, a nebulizer can be, for example, a pneumatic compressor nebulizer, an ultrasonic nebulizer, a vibrating mesh or horn nebulizer, or a microprocessor-controlled breath-actuated nebulizer. In some embodiments, a nebulizer device can be a device as described in, for example, Russian patent RU199823U1.

A nebulizer is a device that turns an active ingredient, such as a 5-HT_{2A} receptor agonist, in solution or suspension into a fine aerosol, such as a mist, for delivery to the lungs. A nebulizer can also be referred to as an atomizer. To atomize is to put a dissolved active ingredient(s) into an aerosol, such as a mist, form. To deliver by nebulization, the active ingredient(s) can be dispersed in a liquid medium, for example, water, ethanol, or propylene glycol. Additionally, the active ingredient(s) can be carried in an excipient such as, for example liposomes, polymers, emulsions, micelles, nanoparticles, or polyethylenimine (PEI). Liquid drug formations for nebulizers c be, for example, aqueous solutions or viscous solutions. After application of a dispersing forcer (e.g., jet of gas, ultrasonic waves, or vibration of mesh), the dissolved active ingredient(s) is contained within liquid droplets, which are then inhaled. A mist can contain liquid droplets containing the active ingredient(s) in gas phase such as air or another gaseous mixture (e.g., a mixture of helium and oxygen, a therapeutic gas mixture containing nitrous oxide, etc.).

Jet nebulizers (also known as pneumatic nebulizers or compressor nebulizers) use compressed gas to make a mist. In some embodiments, a jet nebulizer is a microprocessor-controlled breath-actuated nebulizer, also called a breath-actuated nebulizer. A breath-actuated nebulizer creates a mist only when a patient is inhaling, rather than creating a mist continuously. A mist can be generated by, for example, passing air flow through a Venturi in a nebulizer bowl or cup. A Venturi is a system for speeding the flow of a fluid by constricting fluid in a cone shape tube. In the restriction, the fluid must increase its velocity, thereby reducing its pressure and producing a partial vacuum. As the fluid exits the constriction point, its pressure increases back to the ambient or pipe level pressure. This can form a low-pressure zone that pulls updroplets through a feed tube from a solution of drug in a nebulizer bowl, and in turn this creates a stream of atomized droplets, which flow to a mouthpiece. Higher air flows lead to a decrease in particle size and an increase in output. Due to droplets and solvent that saturates the outgoing gas, jet nebulizers can cool a drug solution in the nebulizer and increase solute concentration in the residual volume. A baffle in a nebulizer bowl or cup can be impacted by larger particles, retaining them and returning them to the solution in the nebulizer bowl or cup to be reatomized. Entrainment of air through a nebulizer bowl as the subject inhales can increase mist output during inspiration. Generation of a mist can occur with a smaller particle size distribution, but using smaller particle sizes can result in an increased nebulization time.

The unit of measurement generally used for droplet size is mass median diameter (MMD), which is defined as the average droplet diameter by mass. This unit can also be referred to as the mass mean aerodynamic diameter, or MMAD. The MMD droplet size for jet nebulizers can be about 1.0, 1.5, 2.0, 2.5, 3.0, 3.5, 4.0, 5.0, 6.0, 7.0, 8.0, 9.0, 10.0 μm or more (or any range between about 1.0 and 10.0 μm), which can be smaller than that of ultrasonic nebulizers.

Ultrasonic nebulizers generate mists by using the vibration of a piezoelectric crystal, which converts alternating current to high-frequency (about 1 to about 3 MHz) acoustic energy. The solution breaks up into droplets at the surface, and the resulting mist is drawn out of the device by the patient's inhalation or pushed out by gas flow through the device generated by a small compressor. Ultrasonic nebulizers can include large-volume ultrasonic nebulizers and small-volume ultrasonic nebulizers. Droplet sizes tend to be larger with ultrasonic nebulizers than with jet nebulizers. The MMD droplet size for ultrasonic nebulizers can be about 2.0, 2.5, 3.0, 3.5, 4.0, 4.5, 5.0, 5.5, 6.0, 6.5, 7.0, 7.5, 8.0, 9.0, 10.0 ram or more (or any range between about 2.0 and 10.0 μm). Ultrasonic nebulizers can create a dense mist, with droplets at about 100, 150, 200, 250, 300 m/L or more.

Mesh nebulizer devices use the vibration of a piezoelectric crystal to indirectly generate a mist. Mesh nebulizers include, for example, active mesh nebulizers and passive mesh nebulizers. Active mesh nebulizers use a piezo element that contracts and expands on application of an electric current and vibrates a precisely drilled mesh in contact with the drug solution to generate a mist. The vibration of a piezoelectric crystal can be used to vibrate a thin metal plate perforated by several thousand holes. One side of the plate is in contact with the liquid to be atomized, and the vibration forces this liquid through the holes, generating a mist of tiny droplets. Passive mesh nebulizers use a transducer horn that induces passive vibrations in the perforated plate with tapered holes to produce a mist. Examples of active mesh nebulizers include the Aeroneb @ (Aerogen, Galway, Ireland) and the eFlow® (PARI, Starnberg, Germany), while the Microair NE-U22 @(Omron, Bannockburn, IL) is a passive mesh nebulizer. Mesh nebulizers are precise and customizable. By altering the pore size of the mesh, the device can be tailored for use with drug solutions of different viscosities, and the output rate changed. Use of this method of atomization can offer several advantages. The size of the droplets can be extremely precise because droplet size can be determined by the size of the holes in the mesh (which may be tailor-made to suit the application). Nebulizer meshes can be manufactured using methods such as electrodeposition, electroplating, and laser cutting to produce a liquid particle in gas in the respirable range. Mesh can be made of metal alloy. The metals used in mesh manufacture can include platinum, palladium, nickel, and stainless steel. The size of the droplet is about twice the size of the mesh hole. Mesh holes, therefore, can be about 0.1, 0.5, 1.0, 1.5, 2.0, 2.5, 3.0, 3.5, 4.0, 4.5, 5.0 μm or more (or any value in between about 0.1 and 5.0 μm). Mist generation in mesh nebulizers can vary based on the shape of the mesh, the material that the mesh is made of, and also the way that the mesh is created. In other words, different meshes can produce different sized liquid particles suspended in gas. Generally, MMD droplet size for mesh nebulizers can be about 1.0, 1.5, 2.0, 2.5, 3.0, 3.5, 4.0, 4.5, 5.0, 5.5., 6.0, 6.1, 6.2, 6.3, 6.4, 6.5, 6.6, 6.7, 6.8, 6.9, 7.0 μm or more (or any value in between about 1.0 and 7.0 μm).

Additionally, droplet size can be programmable. In particular, geometric changes can be made to a nebulizer to provide a specific desired droplet size. Additionally, droplet size can be controlled independently of droplet velocity. The volume of liquid atomized, and the droplet velocity can also be precisely controlled by adjusting the frequency and amplitude of the mesh vibration. Furthermore, the number of holes in the mesh and their layout on the mesh can be tailored. Mesh nebulizers can be powered either by electricity or by battery.

A mist output rate in standing cloud mL per minute (for any atomization methodology described herein) can range from, for example, 0.1, 0.2. 0.3, 0.4, 0.5, 0.6, 0.7, 0.8, 0.9 mL/minute or more (or any range between about 0.1 and 0.9 mL/minute) and the residual volume in any type of nebulizer reservoir can range from a about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mL or more (or any range between about 0.01 and 2.0 mL). Precise droplet size control can be advantageous since droplet size can correlate directly to kinetic drug release (KDR). Precise control of KDR can be achievable with precise control of droplet size. Pharmaceutically acceptable salts of the compounds herein can be delivered via a mist using any methodology with an MMD droplet size of about 0.5, 1.0, 1.5, 2.0, 2.5, 3.0, 3.5, 4.0, 5.0, 6.0, 7.0, 8.0, 9.0, 10.0 μm or more (or any range between about 0.5 and 10.0 m).

In some embodiments, the 5-HT_{2A} receptor agonist and/or the NMDA receptor antagonist can be delivered via a continuous positive airway pressure (CPAP) or other pressure-assisted breathing device. A pressure-assisted breathing device forces a continuous column of compressed air or other gas at a fixed designated pressure against the face and nose of the patient, who is wearing a mask or nasal cap. When the patient's glottis opens to inhale, the pressure is transmitted throughout the airway, helping to open it. When the patient exhales, pressure from the deflating lungs and chest wall pushes air out against the continuous pressure, until the two pressures are equal. The air pressure in the airway at the end of exhalation is equal to the external air pressure of the machine, and this helps “splint” the airway open, allowing better oxygenation and airway recruitment. A pressure-assisted breathing device can be coupled with a means for introducing mist particles into the gas flow in the respiratory circuit and/or a means for discontinuing the introduction of mist particles into the respiratory circuit when the patient exhales. See, e.g. U.S. Pat. No. 7,267,121.

In some embodiments, a mist can be delivered by a device such as a metered dose inhaler (MI) (also referred to as a pressurized metered dose inhaler or pMDI), which generates an organic solvent-droplet mist containing the active ingredient(s), which is optionally combined with a heated helium-oxygen mixture. In some embodiments, the 5-HT_{2A} receptor agonist and/or the NMDA receptor antagonist can be delivered via a metered dose inhaler, MDI. MDI devices can include a canister which contains the 5-HT_{2A} receptor agonist and a propellant, a metering valve which dispenses the medicament from the canister, an actuator body that receives the canister and which forms an opening for oral inhalation, and an actuator stem which receives the drug from the canister and directs it out the opening in the actuator body. A non-limiting example of a metering valve and actuator is Bespak's BK357 valve and actuator (orfice d=0.22 mm) by Reciphanm. Moving the drug canister relative to the actuator body and actuator stem causes the metering valve to release the predetermined amount of the drug. In some embodiments, the 5-HT_{2A} receptor agonist can be dissolved in a liquid propellant mixture (sometimes including small amounts of a volatile organic solvent) stored in a pressurized container of the MDI. The “metered dose” is the dose that is prepackaged in a single-dose inhaler, or which in a multidose inhaler is automatically measured out of a reservoir in preparation for inhalation. MDI devices can be aided with spacers. An MDI spacer is a spacer that goes between the MDI and the mouth of a user of the MI. An MDI spacer allows droplets in the atomized dose to settle out a bit and mix with air or other gas, thus allowing for more effective delivery of a metered dose into a user's lungs when inhaled. An MDI spacer assists in preventing a user from inhaling the metered dose directly from an MDI where the dose would be traveling so fast that the droplets of the atomized spray from the MDI hit and stick to the back of the user's throat rather than being inhaled into the user's lungs where the drug of the metered dose is designed to be delivered. MDI devices offer the advantage of regular dosing, which can be controlled in the manufacture of the drug.

Active ingredient(s) can also be delivered by dry powder inhalers (DPI). In such DPI devices, the active ingredient(s) itself can form the powder or the powder can be formed from a pharmaceutically acceptable excipient or carrier and the active ingredient(s) is releasably bound to a surface of the carrier powder such that upon inhalation, the moisture in the lungs releases the active ingredient(s) from the surface to make available for systemic absorption. The dry powder may contain finely divided powders of the active ingredient(s) and finely divided powders of a pharmaceutically acceptable excipient. Finely divided particles may be prepared by conventional methods known to those of ordinary skill in the art, such as micronization or grinding. In some embodiments, the 5-HT_{2A} receptor agonist is delivered by use of a dry powder inhaler (DPI). The 5-HT_{2A} receptor agonist can be formed into the necessary powder itself (in solid particulate form), or can be releasably bound to a surface of a carrier powder. Such carrier powders are known in the art (see, e.g., H. Hamishehkar, et al., “The Role of Carrier in Dry Powder Inhaler”, Recent Advances in Novel Drug Carrier Systems, 2012, pp. 39-66).

DPI is generally formulated as a powder mixture of coarse carrier particles and micronized drug particles with aerodynamic particle diameters of 1-5 m (see e.g., Iida, Kotaro, et al. “Preparation of dry powder inhalation by surface treatment of lactose carrier particles” Chemical and pharmaceutical bulletin 51.1 (2003): 1-5). Carrier particles are often used to improve particle flowability, thus improving dosing accuracy and minimizing the dose variability observed with active ingredient(s) alone while making them easier to handle during manufacturing operations. Carrier particles desirably have physico-chemical stability, biocompatibility and biodegradability, compatibility with the active ingredient(s), while also being inert, available, and economical. The choice of carrier particle (both content and size) is well within the purview of one of ordinary skill in the art. The most common carrier particles are made of lactose or other sugars, with α-lactose monohydrate being the most common lactose grade used in the inhalation field for such particulate carriers.

Any of the delivery devices above can be optionally manufactured with smart technology enabling remote activation of delivery. The remote activation can be performed via computer or mobile app. To ensure security, the remote activation device can be password encoded. This technology enables a healthcare provider to perform telehealth sessions with a patient, during which the healthcare provider can remotely activate and administer the 5-HT_{2A} receptor agonist, the NMDA receptor antagonist, or both, via the desired delivery device while supervising the patient on the televisit.

Delivery with Helium Oxygen Mixtures

The methods disclosed herein may provide for systemic delivery of the 5-HT_{2A} receptor agonist and/or the NMDA receptor antagonist to a patient's CNS. Doses can be optimized for individual patients' metabolisms and treatment needs. Larger doses with deleterious or undesirable side-effects can be avoided by using small doses of the 5-HT_{2A} receptor agonist and/or the NMDA receptor antagonist. Methods of treating various central nervous system (CNS) diseases and other conditions are described herein. The methods can comprise delivering via inhalation an aerosol, preferably a mist, comprising the 5-HT_{2A} receptor agonist. The NMDA receptor antagonist (e.g., nitrous oxide) can be present in the gas phase of the aerosol, or in a carrier gas used to deliver a generated aerosol to the patient's lungs. The gas phase of the aerosol or the carrier gas can be air, oxygen, helium, a mixture of helium and oxygen (i.e., a heliox mixture), or other gases or other gas mixtures, including therapeutic gas mixtures. In some embodiments, the carrier gas can be heated. The method can further comprise using a device containing a balloon with an oxygen-helium mixture equipped with a reducer and a mask connected to each other by a gas or air connecting tube, which contains an additional heating element capable of heating the gas mixture up to 120° C., a nebulizer with a vibrating porous plate or mesh, ensuring the passage of droplets with a size of less than 5 microns through it, and a disinfection unit.

In some embodiments, the 5-HT_{2A} receptor agonist and/or the NMDA receptor antagonist are delivered to the lower respiratory tract, for instance, to a pulmonary compartment such as alveoli, alveolar ducts and/or bronchioles. From there, the active ingredient(s) can enter the blood stream and travel to the central nervous system. Administration via inhalation, e.g., as a mist, can deliver the active ingredient(s) to the patient's CNS without passing through the liver. Administration via inhalation can allow gaseous drugs such as nitrous oxide or those dispersed in a liquid or a mist, to be rapidly delivered to the blood stream, bypassing first-pass metabolism. First-pass metabolism, also known as “first-pass effect” or “presystemic metabolism” describes drugs that enter the liver and undergo extensive biotransformation.

In some embodiments, the present disclosure provides a treatment step, in which a patient in need thereof is administered via inhalation a gas phase, e.g., a mixture of helium and oxygen, heated to about 50° C., 51° C., 52° C., 53° C., 54° C., 55° C., 56° C., 57° C., 58° C., 59° C., 60° C., or more (or any range between 50° C. to 60° C.) and the atomized 5-HT_{2A} receptor agonist. In some embodiments, an aerosol (e.g., a mist), or vapor of the 5-HT_{2A} receptor agonist can have a particle size from about 0.1 microns to about 10 microns (e.g., about 10, 5, 4, 3, 2, 1, 0.1 or less microns). In some embodiments, the 5-HT_{2A} receptor agonist can be atomized via a nebulizer creating an inhalant that is a mist. In some embodiments, the atomized 5-HT_{2A} receptor agonist is driven down the patient delivery line by the patient's inhalation. In some embodiments, the atomized 5-HT_{2A} receptor agonist is driven down the patient delivery line by the patient's inhalation using a carrier gas. The carrier gas can be air, oxygen, a mix of oxygen and helium, heated air, heated oxygen, a heated helium and oxygen mixture, among others. The carrier gas can also be a therapeutic gas mixture, for example, containing nitrous oxide as the NMDA receptor antagonist.

In some embodiments, the treatment step c be preceded by a pretreatment step. In some embodiments, the pretreatment step can comprise first administering a pretreatment inhalation therapy prior to administration of the mist of the 5-HT_{2A} receptor agonist. In some embodiments, the pretreatment inhalation step can comprise (i) administering via inhalation air, oxygen, or mixture of helium and oxygen heated to about 90° C., 911° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., or more (or any range between about 90° C. and 120° C.) and no active ingredient(s), and then (ii) administering a treatment step of inhalation air, oxygen, a mix of oxygen and helium, heated air, heated oxygen, or heated helium and oxygen mixture and the atomized 5-HT_{2A} receptor agonist. Heated air, heated oxygen, or heated helium and oxygen mixture, in combination with the atomized 5-HT_{2A} receptor agonist, can be heated to about 50° C., 51° C., 52° C., 53° C., 54° C., 55° C., 56° C., 57° C., 58° C., 59° C., 60° C., or more (or any range between about 50° C. and 60° C.). In the treatment step (ii), the NMDA receptor antagonist (e.g., nitrous oxide) can also optionally be present in the air, oxygen, a mix of oxygen and helium, heated air, heated oxygen, or heated helium and oxygen mixture gas phase of the aerosol, or can be present in a carrier gas used to entrain the aerosol and deliver to the patient.

In some embodiments, a pretreatment step (i) and a treatment step (ii) can be repeated 0, 1, 2, 3, 4, 5, or more times. In some embodiments, steps (i) and (ii) can be repeated 0, 1, 2, 3, 4, 5, or more times followed by the treatment step, which can be repeated 0, 1, 2, 3, 4, 5, or more times. In some embodiments, the treatment step can be repeated 0, 1, 2, 3, 4, 5, or more times with no pretreatment step.

Treatment, with optional pretreatment, can be administered once a week, twice a week, once a day, twice a day, three times a day or more, and other treatment regimens as set forth herein, such as 2 to 8 treatment session per treatment course. Each treatment (i.e., inhalation session) can be for about 1, 5, 10, 20, 30, 45, 60 or more minutes.

A drug delivery procedure can comprise an inhaled priming no-drug hot heliox mixture to effectively preheat the mucosal bed followed by inhaling an atomized 5-HT_{2A} receptor agonist, again driven by the heated heliox, with or without nitrous oxide, but at lower temperatures, that are now dictated by lower heat tolerance to the wet vs. dry inhaled gas stream. Consequently, this procedure can be conducted in multiple repeated cycles, wherein a target PK and drug exposure is controlled by the concentration of the active ingredient(s), temperature, flow rate of the helium oxygen mixture, composition of the mixture, number and durations of cycles, time and combinations of the above.

Methods of delivery described herein can be used to treat certain diseases and disorders, such as those set forth herein, including a central nervous system (CNS) disorder or psychological disorder, comprising administering via inhalation a heated mixture of helium and oxygen heated and an atomized 5-HT_{2A} receptor agonist, optionally together with an NMDA receptor antagonist (e.g., nitrous oxide), e.g., in a therapeutic gas mixture. The treatment can alleviate one or more symptoms of the disorder.

In some embodiments, the 5-HT_{2A} receptor agonist can be administered for treatment of CNS disease or other disorder. In some embodiments, the 5-HT_{2A} receptor agonist can be administered to treat depression including, but not limited to major depression, melancholic depression, atypical depression, or dysthymia. In some embodiments the 5-HT_{2A} receptor agonist can be administered to treat psychological disorders including anxiety disorder, obsessive compulsive disorder, addiction and substance abuse disorders (e.g., narcotic addiction, tobacco addiction, opioid addiction, alcoholism), depression and anxiety (chronic or related to diagnosis of a life-threatening or terminal illness), compulsive behavior, or a related symptom.

In some embodiments, the disease or disorder can include central nervous system (CNS) disorders and/or psychological disorders, including, for example, post-traumatic stress disorder (PTSD), major depressive disorder (MDD), treatment-resistant depression (TRD), suicidal ideation, suicidal behavior, major depressive disorder with suicidal ideation or suicidal behavior, melancholic depression, atypical depression, dysthymia, non-suicidal self-injury disorder (NSSID), bipolar and related disorders (including, but not limited to, bipolar I disorder, bipolar II disorder, cyclothymic disorder), obsessive-compulsive disorder (OCD), generalized anxiety disorder (GAD), acute psychedelic crisis, social anxiety disorder, substance use disorders (including, but not limited to, alcohol use disorder, opioid use disorder, amphetamine use disorder, nicotine use disorder, and cocaine use disorder), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, chronic fatigue syndrome, Lyme disease, gambling disorder, eating disorders (including, but not limited to, anorexia nervosa, bulimia nervosa, binge-eating disorder, etc.), and paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido), and obesity. In some embodiments, the disease or disorder may include conditions of the autonomic nervous system (ANS). In some embodiments, the disease or disorder may include pulmonary disorders (e.g., asthma and chronic obstructive pulmonary disorder (COPD). In some embodiments, the disease or disorder may include cardiovascular disorders (e.g., atherosclerosis).

The methods of administering the 5-HT_{2A} receptor agonist and the N-methyl-D-aspartate (NMDA) receptor antagonist via inhalation, such as through a nebulizer or other device as described herein (including, for example, using a heated helium-oxygen mixture), can lead to advantageous improvements in multiple PK parameters as compared to oral delivery. In particular, the 5-HT_{2A} receptor agonist delivered via inhalation can cross the blood brain barrier and be delivered to the brain. As compared to oral delivery, the method of administering the 5-HT_{2A} receptor agonist to the patient via inhalation, such as with a nebulizer or other device as described herein, optionally with a heated heliox mixture, can increase bioavailability by at least 25% as compared to oral delivery. In some embodiments, the method of administering the 5-HT_{2A} receptor agonist to the patient via inhalation, such as with a nebulizer or other device as described herein, can increase bioavailability by about 10%, 25%, 30%, 35%, 40%, 50%, 55%, 60%, 65%, 70%, 80%, 85%, 90%, 95%, 99%, 99.9%, or more. The method of administering the 5-HT_{2A} receptor agonist to the patient via nebulizer as described herein, can reduce Tₘₐₓ by at least 50% as compared to oral delivery. In some embodiments, the method of administering the 5-HT_{2A} receptor agonist to the patient via nebulizer as described herein, can reduce Tₘₐₓ by at 30%, 40%, 50%, 55%, 60%, 65%, 70%, 80%, 85%, 90%, 95%, 99%, 99.9%, or more. In some embodiments, the method of administering the 5-HT_{2A} receptor agonist to the patient via nebulizer or other device as described herein, can increase Cₘₐₓ by at least 25% as compared to oral delivery. In some embodiments, the method of administering the 5-HT_{2A} receptor agonist to the patient via nebulizer or other device as described herein, can increase Cₘₐₓ by about 10%, 25%, 30%, 35%, 40%, 50%, 55%, 60%, 65%, 70%, 80%, 85%, 90%, 95%, 99%, 99.9%, or more. Furthermore, a method of administering the 5-HT_{2A} receptor agonist to the patient via inhalation using a nebulizer or other device as described herein, can allow clinical protocols enabling dose titration and more controlled exposure. Controlled exposure enables adjusting the patient experience and providing overall improved therapeutic outcomes. With the smart technology enabled devices for inhalation delivery noted above, the dose titration and controlled delivery can be performed remotely by the healthcare worker, enabling the patient to be in the comfort of their own home, improving the patient's experience and outcome.

In some embodiments, a system is provided for administering the 5-HT_{2A} receptor agonist that includes a container comprising a solution of the 5-HT_{2A} receptor agonist and a nebulizer physically coupled or co-packaged with the container and adapted to produce an aerosol, preferably a mist, of the solution having a particle size from about 0.1 microns to about 10 microns (e.g., about 10, 5, 4, 3, 2, 1, 0.1 or less microns). The system may also include a blending system and/or pressurized tanks/canisters of a therapeutic gas mixture comprising the NMDA receptor antagonist (nitrous oxide) that can be fluidly connected to the nebulizer for generation of an aerosol, preferably a mist, or used as a carrier gas to aid delivery of the aerosol.

The combination of the 5-HT_{2A} receptor agonist and NMDA receptor antagonist administered via the inhalation route may lead to greater therapeutic efficacy than is achievable with maximum tolerable doses of either class of active ingredient used independently. Thus, these active ingredients may be employed in lesser doses to provide a therapeutic effect that is equivalent to that of larger doses of individual agent. Accordingly, by combining both the 5-HT_{2A} receptor agonist and the NMDA receptor antagonist via the inhalation route, the benefits of each class may be achieved without the undesirable psychiatric adverse effects and potential toxicities.

In some embodiments, the delivery device is an inhalation delivery device for delivery of the combination of the 5-HT_{2A} receptor agonist (e.g., DMT, 5-MeO-DMT, DMT-d₁₀, 5-MeO-DMT-d₁₀, etc.) and nitrous oxide by inhalation to a patient in need thereof, comprising an inhalation outlet portal for administration of the combination to the patient; a container configured to deliver nitrous oxide, e.g., in a therapeutic gas mixture, to the inhalation outlet portal; and a device configured to generate and deliver an aerosol comprising the 5-HT_{2A} receptor agonist to the inhalation outlet portal. In some embodiments, the inhalation outlet portal is selected from a mouthpiece or a mask covering the patient's nose and mouth. In some embodiments, the device configured to generate and deliver the aerosol to the inhalation outlet portal is a nebulizer. In some embodiments, the nebulizer is a jet nebulizer and the nitrous oxide gas, alone, or in combination with other gases (therapeutic gas mixture containing nitrous oxide), acts as a driving gas for the jet nebulizer. Accordingly, nitrous oxide delivered using a nebulizer (e.g., jet nebulizer) may dually act as a therapeutic agent and as a driving gas to entrain the nebulized form of the 5-HT_{2A} receptor agonist. In some embodiments, the device further comprises smart technology, e.g., electronics, configured to provide remote activation and operational control of the inhalation delivery device as noted above.

In some embodiments, the device is a dual delivery device configured to administer the 5-HT_{2A} receptor agonist, preferably in the form of an aerosol, and to simultaneously administer a controlled amount of nitrous oxide, either alone or as a therapeutic gas mixture. Any of the above aerosol delivery devices can be used for such a device, with the addition of a source of nitrous oxide (or a source of a therapeutic gas mixture containing nitrous oxide) configured to provide a metered, controlled dose/flow rate of nitrous oxide through the same administration outlet as the aerosol delivery device. In some embodiments, the driving gas for the nebulization of the 5-HT_{2A} receptor agonist is the nitrous oxide or therapeutic gas mixture containing nitrous oxide.

Fast-acting combination drug therapies can also be selected through selection of 5-HT_{2A} receptor agonists with a short elimination half-life (t_{1/2}) and selection of a fast-acting NMDA receptor antagonist such as nitrous oxide. In some embodiments, the 5-HT_{2A} receptor agonists is selected which has an elimination half-life (t_{1/2}) of less than 2 hours, e.g., from 0.1 minutes to 120 minutes, 0.5 minutes to 110 minutes, 1 minutes to 100 minutes, 2 minutes to 80 minutes, 3 minutes to 70 minutes, 4 minutes to 60 minutes, 5 minutes to 50 minutes, 6 minutes to 40 minutes, 7 minutes to 35 minutes, 8 minutes to 30 minutes, 9 minutes to 25 minutes, 10 minutes to 20 minutes, 12 minutes to 18 minutes, 14 minutes to 16 minutes, or about 15 minutes. Preferably, the 5-HT_{2A} receptor agonist is a short-acting psychedelic that has an elimination half-life of less than 90 minutes, less than 75 minutes, less than 60 minutes, less than 45 minutes, less than 30 minutes, less than 25 minutes, or less than 20 minutes.

In some embodiments, the 5-HT_{2A} receptor agonist used in the fast-acting therapeutic combination is a compound having at least one deuterium atom, for example, a tryptamine derivative of Formula (I), Formula (II), Formula (II-a), Formula (II-b), Formula (II-c), Formula (II-d), comprising at least one deuterium atom, a phenethylamine derivative of Formula (III), Formula (III-a), Formula (IV), Formula (IV-a), Formula (IV-b), Formula (V), Formula (V-a), Formula (V-b), Formula (VI), Formula (VI-a), Formula (VI-b), comprising at least one deuterium atom, or a combination thereof. In preferred embodiments, the 5-HT_{2A} receptor agonist of the fast-acting therapeutic combination is at least one selected from the group consisting of N,N-dimethyltryptamine (DMT), 5-methoxy-N,N-dimethyltryptamine (5-MeO-DMT), and deuterated analogs thereof such as DMT-d₁₀(2-(1H-indol-3-yl)-N,N-bis(methyl-d₃)ethan-1-amine-1,1,2,2-d₄) and 5-MeO-DMT-d₁₀ (2-(5-methoxy-1H-indol-3-yl)-N,N-bis(methyl-d₃)ethan-1-mine-1,1,2,2-d₄). Most preferably, the 5-HT_{2A} receptor agonist of the fast-acting therapeutic combination is DMT. A short-acting psychedelic, such as DMT and 5-MeO-DMT, has an elimination half-life of about 12 to 19 minutes.

Regarding the fast-acting NMDA receptor antagonist, nitrous oxide, in particular, gives a rapid onset of effects yet is quickly removed from the body-its effects cease almost immediately upon removal e.g., when the flow of gas is stopped. Nitrous oxide is thus compatible with the aforementioned short-acting 5-HT_{2A} agonists including DMT, 5-MeO-DMT, and the deuterated analogs thereof, in the fast-acting therapeutic combination disclosed herein.

The aforementioned fast-acting therapeutic combination may be advantageous for acute treatment applications, such as to treat acute psychiatric conditions e.g., as a rescue medicine when someone is suicidal. The therapeutic combination may be especially useful to treat acute conditions that require a quick onset of effect, a short duration of action and minimal psychiatric adverse effects. Non-limiting examples of acute psychiatric conditions include, but are not limited to, suicidal ideation and suicide attempts, social anxiety disorder, drug withdrawal, post-traumatic stress disorder (PTSD), and panic attacks.

The fast-acting therapeutic combination that includes nitrous oxide and a short-acting 5-HT_{2A} receptor agonist may be formulated and administered as specified previously. For example, nitrous oxide may be administered using a blending system that combines N₂O, air or O₂, and optionally other gases from separate compressed gas cylinders into a therapeutic gas mixture which is delivered to a patient via inhalation. Alternatively, the therapeutic gas mixture containing N₂O, air or O₂, and optionally other gases may be packaged, for example, in a pressurized tank or in small pressurized canisters. N₂O may be titrated in the therapeutic gas mixture at a concentration ranging from 5 vol % to 75 vol %, from 10 vol % to 50 vol %, from 15 vol % to 40 vol % relative to a total volume of the therapeutic gas mixture. The therapeutic gas mixture may be administered for up to 3 hours, up to 2 hours, up to 90 minutes, up to 60 minutes, or up to 30 minutes, e.g., from at least 1 minute, at least 2 minutes, at least 5 minutes, at least 10 minutes, at least 15 minutes, at least 25 minutes. In addition, the short-acting 5-HT_{2A} receptor agonist may be administered as any suitable pharmaceutical composition, e.g., capsules, tablets, pills, pellets, lozenges, powders, granules, syrups, elixirs, solutions, suspensions, emulsions, suppositories, or sustained-release formulations thereof. A suitable dose of the short-acting 5-HT_{2A} receptor agonist may be within the dosage range described previously, however, in some embodiments, the suitable dose of the short-acting 5-HT_{2A} receptor agonist may fall outside of the given range. When DMT is used as the short-acting 5-HT_{2A} receptor agonist, an effective amount of DMT may range from 10 to 100 mg, for example.

Nitrous oxide and the fast-acting 5-HT_{2A} receptor agonist in the fast-acting therapeutic combination may be administered sequentially, concurrently but separately, or concurrently as a single composition. In some embodiments, the fast-acting therapeutic combination may be in the form of an aerosol or dry powder dispersion for inhalation, preferably in the form of an aerosol (e.g., mist) for inhalation. The nitrous oxide may be administered concurrently with the fast-acting 5-HT_{2A} receptor agonist via an aerosol inhalation. Accordingly, nitrous oxide may dually act as a propellant gas for the aerosol generation or as a carrier gas to facilitate delivery of a generated aerosol, and as an active ingredient of the fast-acting therapeutic combination.

The fast-acting therapeutic combination of the present disclosure may be used for treatment of an acute psychiatric condition in a subject in need thereof. In such treatment methods, the fast-acting therapeutic combination is typically administered for a time period of less than or equal to the elimination half-life of the 5-HT_{2A} receptor agonist of the combination.

The present disclosure also relates to a rescue medicine kit that contains the fast-acting therapeutic combination (e.g., nitrous oxide and the fast-acting 5-HT_{2A} receptor agonist). The rescue medicine kit may include containers in unit dosage form or multi-dosage form of each active ingredient. In a unit dosage form, the preparation is subdivided into unit doses containing appropriate quantities of the active ingredient(s). The unit dosage form can be a packaged preparation, the package containing discrete quantities of preparation, such as packeted tablets, capsules, and powders in vials or ampoules. Also, the unit dosage form can be a single-dose inhaler, capsule, tablet, cachet, or lozenge, or a plurality of any of these in packaged form, for example, a plurality of single-dose inhalers. Multi-dosage forms include a metered multi-dose inhaler that is automatically measured out of a reservoir in preparation for inhalation. In some embodiments, the rescue medicine kit includes a container comprising nitrous oxide, a solution of the short-acting 5-HT_{2A} receptor agonist formulation, and a nebulizer physically coupled or co-packaged with the kit and adapted to produce an aerosol mist of the fast-acting therapeutic combination. Such unit dosage forms can be administered, for example, by emergency responders, with minimal side effects to the patient.

### EXAMPLES

I. DMT and DMT-d₁₀: Pharmacokinetic Study by Intravenous (bolus), Oral Gavage and Inhalation Administration to Male Rats

The pharmacokinetics and bioavailability of N,N-dimethyltryptamine (DMT) and 2-(1H-indol-3-yl)-N,N-bis(methyl-d₃)ethan-1-amine-1,1,2,2-d₄ (DMT-d₁₀) were investigated in rats following intravenous (bolus), oral gavage (OG), and inhalation after co-dose administration. The experimental conditions and results are presented below.

Animals. Twenty-nine male Sprague Dawley rats aged 7-10 weeks and weighing between 210-290 g at dosing were used. Animals were supplied by a recognized supplier of laboratory animals.

Housing. The in-life experimental procedures were subject to the provisions of the United Kingdom Animals (Scientific Procedures) Act 1986 Amendment Regulations 2012 (the Act). The number of animals used were the minimum that is consistent with scientific integrity and regulatory acceptability, consideration having been given to the welfare of individual animals in terms of the number and extent of procedures to be carried out on each animal.

Animals were uniquely identified by tattoo or by microchip. During the pre-trial holding periods, the animals were group housed in caging appropriate to the species. Rats were housed 3 per cage with access to food (Teklad 2014C, pelleted diet) and quality tap water ad libitum, Animals were checked regularly throughout the duration of the study. Any clinical signs were closely monitored and recorded.

There was limited access to animal facility to minimize of external biological and chemical agents. Air supply was filtered and not re-circulated. Temperature and humidity were within the ranges of 20-24° C. and 40-70%, respectively. Lighting was 12 hours light; 12 hours dark.

Test Items. DMT (fumarate salt) and DMT-d₁₀. Both test items were formulated as solutions in vehicle. The vehicle used was citrate (0.1 M) buffer, pH 6.0. To prepare the vehicle, citric acid monohydrate+trisodium citrate dihydrate were weighed into a suitable sized container, dissolved in ca. 90% of final volume of water for injection (WFI), and magnetically stirred to mix. The pH was checked and adjusted to 6.0±0.1 using NaOH or HCl, and the strengths and volumes were recorded. The final volume was made with WFI, and magnetically stirred to mix. The vehicle was then filtered through a 0.22 μm PVDF filter. Some vehicle was dispensed into the appropriate containers for the control group prior to starting the test formulations, with sampling performed at this point, if required. The test item was acclimated to room temperature before use and weighed in the required amount (weighing may be performed in advance). ca. 50% of the final volume of vehicle was added to the test item to obtain a solution, washing the container containing both test item weighing's. An initial mix, with crushing any large particles, may be made by hand using a spatula. If required, the mixture was transferred to a larger container. Dissolution and mixing were performed using a magnetic stirrer, and the start and finish times were recorded. Sonication was used to aide in dissolution if needed. The pH was checked and adjusted to 6.0±0.1 with NaOH or HCl. Strengths and volumes were recorded. The test item solutions were transferred to a measuring cylinder and made up to final volume with remaining vehicle and stirred for a minimum of 20 minutes using a magnetic stirrer. The final pH was checked and recorded (adjusted if necessary), as was the osmolarity. Sampling was performed at this point, if required, whilst magnetically stirring. The solutions were transferred to final containers, via syringe, whilst magnetically stirring.

The following salt correction factors were used:
i. 1.62 for DMT (fumarate)
ii. 1.5 for DMT-d₁₀(free base)
iii. 1.67 for DMT-d₁₀ (fumarate)

### Nominal Co-Administration Dose Levels.

IV and oral: DMT+DMT-d₁₀: 1.62 mg/mL+1.05 mg/kg
Inhalation: DMT+DMT-d₁₀: 81.0 mg/mL+83.5 mg/kg

Experimental Design. This was a single use study with 4 treatment groups as outlined in Table 1.

TABLE 1
Treatment Groups-Co-Administration of DMT and DMT-d₁₀

| Group | Number | | | Dose | Animal |
|---|---|---|---|---|---|
| # | and Sex | Dose route | Treatments | (mg/kg)^{b)} | Numbers |
| 1ª) | 3M | Intravenous | DMT + DMT-d₁₀ | 1 + 1 | 1-3 |
| | | (bolus) | | | |
| 2 | 3M | Oral gavage | DMT + DMT-d₁₀ | 10 + 10 | 4-6 |
| 4 | 12M | Intravenous | DMT + DMT-d₁₀ | 1 + 1 | 14-25 |
| | | (bolus) | | | |
| 6 | 3M | Inhalation | DMT + DMT-d₁₀ | 15.3 + 14.7 | 28-30 |

^{a)}After the IV administration to Group 1, there were blood sampling technical difficulties that resulted in an inadequate number of samples to determine PK parameters. For this reason, PK parameters for Group 1 are not reported.
^{b)}Co-administration of DMT and DMT-d10; free base dose levels

Animals received a single IV bolus via the lateral tail vein, or an oral dose via flexible gavage tube. For the inhalation dose, animals were placed in an inhalation chamber and received a 20-minute aerosolized exposure. Bodyweights were recorded for each animal prior to dosing.

### Inhalation Procedure.

Pre-study characterization. Before commencement of treatment, the system was characterized at the target aerosol concentrations without animals in order to demonstrate satisfactory particle size, satisfactory operation of the exposure system, and reproducibility of test item concentration.

Test atmosphere generation. A suitable nebulizer (or multiple nebulizers) was used to deliver the inhalation dose. The test substance liquid formulation was added to the reservoir of the nebulizer in bulk or added to the reservoir at a controlled rate by syringe driver. Precise details of the operating conditions were determined to achieve the target droplet aerosol concentrations.

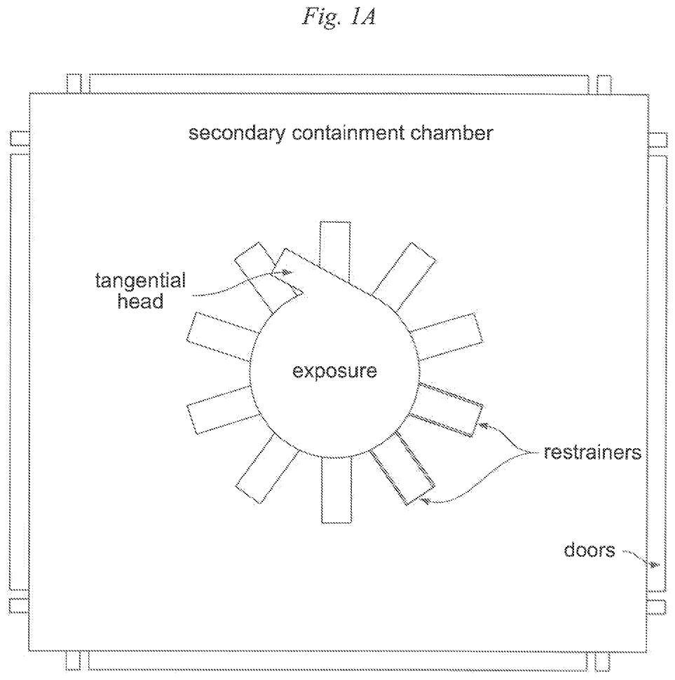

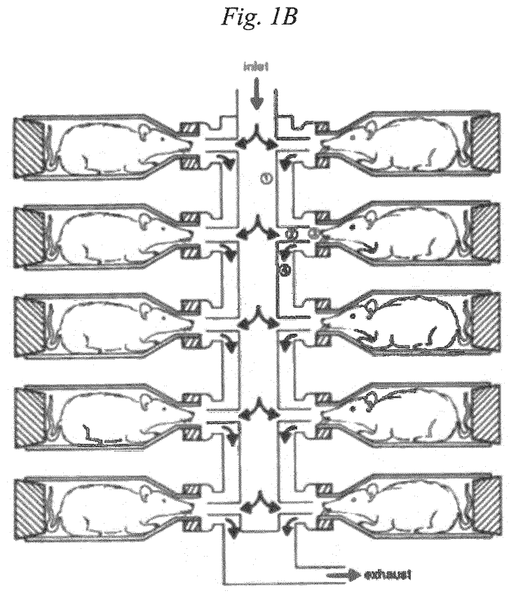

Test atmosphere administration. The inhalation dose was received by snout only exposure. The equipment was a directed flow exposure chamber with modular construction in aluminum alloy comprising a base unit, a variable number of sections each having 8 exposure ports, and a top section incorporating a central aerosol inlet with a tangential air inlet. During exposure, the rats were held in restraining tubes with their snouts protruding from the ends of the tubes into the exposure chambers. Animal exposure ports not in use were closed with blanking plugs. The exposure system was housed in an extract cabinet/secondary containment chamber. The animals on study were acclimated to the method of restraint over at least a 3-day period prior to dosing. The duration of exposure was determined to be 20 minutes. A representation of the directed flow exposure chamber is shown in FIGS. 1A-1B.

Test atmosphere analysis. The inhalation amount of DMT and DMT-d₁₀ were determined from samples collected on filters by gravimetric analysis and the concentration calculated. The particle size of DMT was determined on collections from glass fibre filters. From these data, the mass medium aerodynamic diameter (MMAD) and the geometric standard deviation (ag) of the aerosol was calculated assuming a log-normal distribution of particle size. The inhalation dose in mg/kg was determined according to equation (1):

$$Dose ( mg / kg ) = \frac{C ( μg / L ) \times RMV ( L / min ) \times D ( min )}{BW ( kg ) \times 1000} \qquad ( 1 )$$

where:
   C=Aerosol concentration (μg/L).
   RMV=Respiratory minute volume=0.608×BW^{0.852}
   D=Duration of exposure (20 mins).
   BW=Body weight (kg).

Sampling collection. PK samples (0.3 mL) were collected from the jugular vein by venepuncture into tubes containing K₂EDTA anticoagulant at the following sampling times: Group 1 (IV) and Group 2 (oral) serial plasma collection at 0.083, 0.25, 0.5, 1, 3, 8 and 24 hr postdose; Group 4 (IV) composite plasma and brain collection at 0.083, 0.25, 0.5 and hr postdose; Group 6 (inhalation) serial plasma collection at 0.333, 0.533, 0.833, 1.333, 3.333, 8.333 and 24.333 after start of inhalation.

Plasma samples: Immediately following collection, samples were inverted to ensure mixing with anti-coagulant and placed on wet ice. Plasma was generated by centrifugation (2000 g, 10 min, 4° C.) within 60 min of collection. 90 μL of plasma was transferred into a tube containing 90 μL (1:1 (v/v)) of 200 mM ascorbic acid. Three 50 μL of stabilized plasma samples were aliquoted into polypropylene tubes, frozen on dry ice and stored in −70° C. (±10° C.) until analysis.

Brain samples: After extraction of whole brain from the cranium, brains were rinsed, patted dry, weighed, placed into tubes and frozen on dry ice. Thereafter, they were stored at −70 (10°) C pending analysis.

Bioanalysis. Plasma and brain homogenates were analyzed for DMT and DMT-d₁₀ using an established LC-MS/MS assay.) Pharmacokinetic parameters were determined from the DMT and DMT-d₁₀ plasma and brain concentration-time profiles using commercially available software (Phoenix® WinNonlin®).

### Results.

After IV dose administration to Group 1, there were sampling technical difficulties that prevented an adequate number of collections to construct reliable concentration-time profiles. For this reason, PK parameters for Group 1 are not presented.

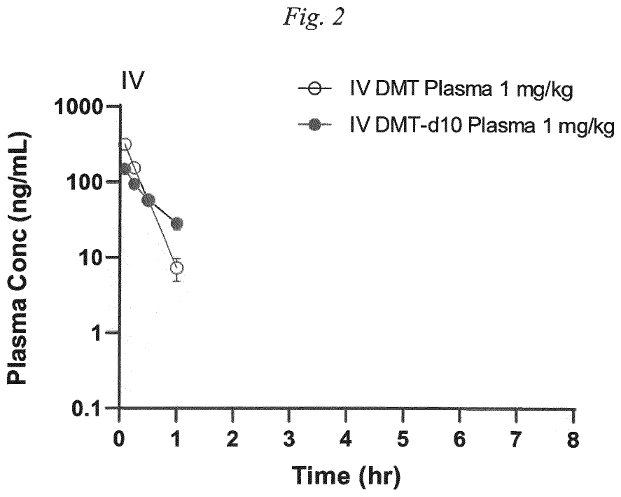

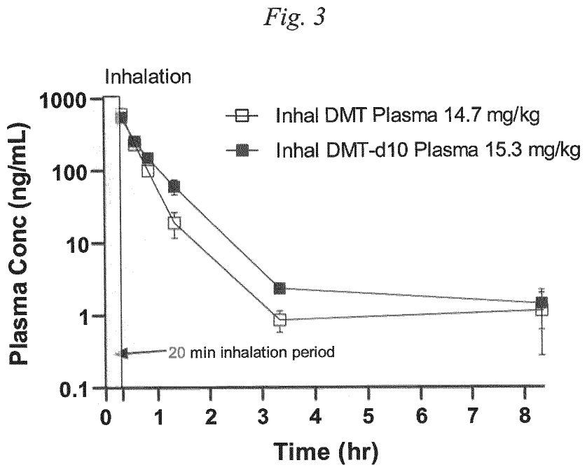

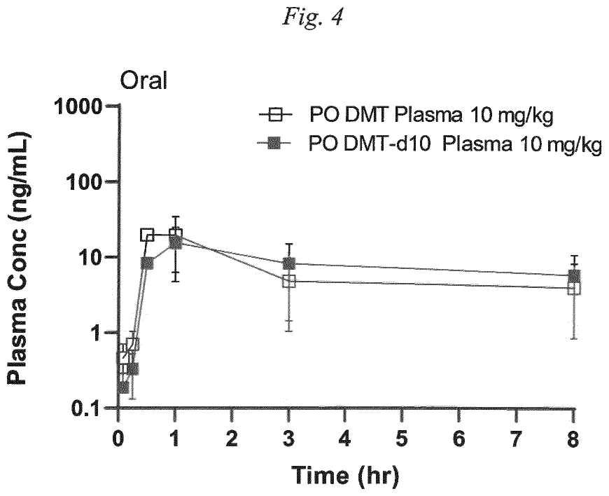

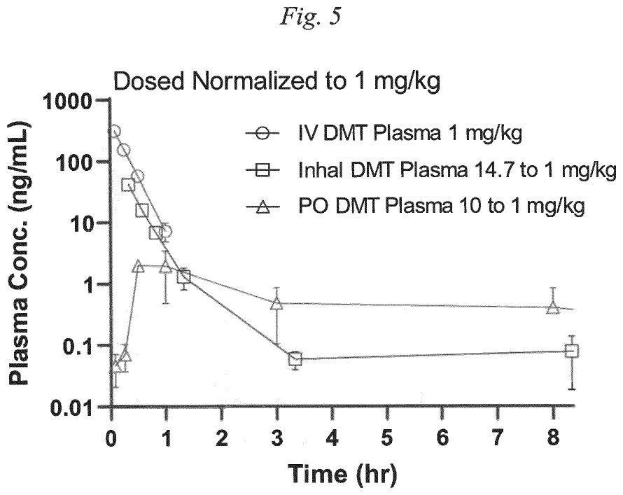

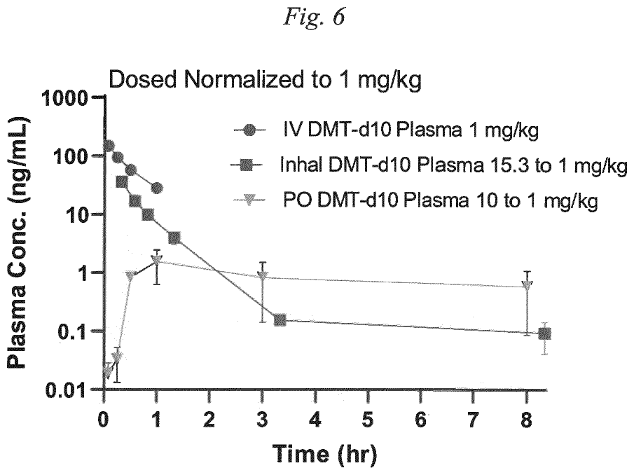

Group 4 replaced and expanded Group 1 with the simultaneous collection of plasma and brain after IV co-administration of DMT and DMT-d₁₀. The mean plasma and brain PK parameters are summarized in Tables 2 and 3, respectively. Group 2 (oral) and Group 6 (inhalation) PK parameters are summarized in Table 2. The PK parameters used to calculate brain to plasma ratios and bioavailability (% F) after oral and inhalation administration of DMT and DMT-d₁₀ are shown in Table 4. The DMT and DMT-d₁₀ plasma concentration-time profiles after IV, inhalation, and oral administration are shown in FIGS. 2, 3, and 4, respectively. FIGS. 5 and 6 represent DMT and DMT-d₁₀ plasma concentration-time profiles normalized to a 1 mg/kg dose, respectively.

Co-administrated doses of DMT and DMT-d₁₀ were 1±1 mg/kg for IV; 10+10 mg/kg for oral and 15.3+14.7 mg/kg for inhalation, respectively. Examination of the plasma concentration-time DMT and DMT-d₁₀ profiles illustrate that plasma exposure after inhalation was as rapid as an IV bolus, with the highest concentrations observed at the first time points taken, 0.333 and 0.083 hr, respectively. Corresponding Cₘₐₓ values of DMT and DMT-d₁₀ were 314 and 148 ng/n after IV and 616 and 554 ng/mL after inhalation, respectively. In contrast, peak plasma concentrations after oral administration, were achieved 1 hr postdose, with Cₘₐₓ values of 28.0 and 20.8 ng/mL, DMT and DMT-d₁₀, respectively. Matched and dose normalized integrated exposures (AUC₀₋₁/dose) were used to calculate bioavailabilities (% F) of DMT and DMT-d₁₀: 15.3 and 24.3% after inhalation and 1.2 and 1.2% after oral exposure, respectively. The mean residence time (MRT) was approximately 2× greater after inhalation compared to IV administration.

Distribution of DMT and DMT-d₁₀ into brain was high. Brain Cₘₐₓ values were 3430 and 1490 ng/g, respectively, compared to their matched plasma concentrations of 314 and 148 ng/mL, respectively.

Deuteration improved the brain to plasma (B/P) ratio by approximately 50% (14 vs 9; DMT-d₁₀ vs. DMT, respectively); improved the duration of exposure (MRTₗₐₛₜ) by 29 to 53% after inhalation and IV; and increased inhalation bioavailability by approximately 60% (24.3% vs. 15.3%, DMT-d₁₀ vs. DMT, respectively), approximately 20× greater than oral bioavailability.

TABLE 2
Plasma pharmacokinetic parameters

| | Dose | Dose Level | | Cₘₐₓ | Tₘₐₓ | AUC₀₋ₜ | AUC_{0-inf} | AUC₀₋₁ | t_{1/2}^{b)} | F^{c)} |
|---|---|---|---|---|---|---|---|---|---|---|
| Analyte | Groupª) | (mg/kg) | Sex | (ng/mL) | (h) | (h*ng/ml) | (h*ng/ml) | (h*ng/ml) | (h) | (%) |
| DMT | 2 (OG) | 10 | Male | 28.0 | 1.00 | 65.0 | NR | 13.7 | NR | 1.2 |
| DMT | 4 (IV) | 1 | Male | 314 | NA | 113 | 115 | 113 | 0.169 | NA |
| DMT | 6 (Inh) | 15.3 | Male | 616 | 0.333 | 300 | 301 | 265 | 0.329 | 15.3 |
| DMT-d₁₀ | 2 (OG) | 10 | Male | 20.8 | 1.00 | 77.2 | NR | 8.78 | NR | 1.2 |
| DMT-d₁₀ | 4 (IV) | 1 | Male | 148 | NA | 74.6 | 92.6 | 74.6 | 0.441 | NA |
| DMT-d₁₀ | 6 (Inh) | 14.7 | Male | 554 | 0.333 | 361 | 363 | 267 | 0.395 | 24.3 |

^{a)}There were sampling technical difficulties with Group 1 that prevented an adequate number of collections to construct reliable concentration-time profiles. For this reason, PK parameters for Group 1 are not presented.
^{b)}Plasma t_{1/2} values calculated: Group 4 (IV), 0.083 to 1.0 hr. Group 6 (Inh), 0.333 to 3.333 hr.
^{c)}Calculated from dose normalized AUC₀₋₁ mean values; Group 4 IV; Group 2 oral; Group 6 inhalation
NA = Not applicable;
NR = Not reportable due to an inability to construction a plasma concentration-time profile or characterize the elimination phase.

TABLE 3
Brain pharmacokinetic parameters

| | | Dose | | | | | | |
|---|---|---|---|---|---|---|---|---|
| | | Level | | | | AUC₀₋ₜ | | |
| | Dose | (mg/kg/ | | Cₘₐₓ | Tₘₐₓ | (h*ng/ | AUC_{0-inf} | t_{1/2}ª⁾ |
| Analyte | Group | day) | Sex | (ng/g) | (h) | g) | (h*ng/g) | (h) |
| DMT | 4 (IV) | | Male | 3430 | 0.0833 | 1060 | 1070 | 0.155 |
| DMT- | 4 (IV) | | Male | 1490 | 0.0833 | 931 | 1290 | 0.565 |
| d₁₀ | | | | | | | | |

^{a)}Brain t_{1/2} values calculated: Group 4 (IV), 0.25 to 1 hr

TABLE 4
Summary pharmacokinetic parameters

| | Dose (mg/kg) | | | | | | | | | | | |
|---|---|---|---|---|---|---|---|---|---|---|---|---|
| | | DMT = | AUC₀₋₁ (hr*ng/mL) | | AUC₀₋₁/dose | | t_{1/2} (hr)^{a)} | | MRTlast (hr) | | B/P (AUC₀₋₁) | |
| Route | DMT | d₁₀ | DMT | DMT-d₁₀ | DMT | DMT-d₁₀ | DMT | DMT-d₁₀ | DMT | DMT-d₁₀ | DMT | DMT-d₁₀ |
| Intravenous | 1 | 1 | 113 | 74.6 | 113 | 74.6 | 0.169 | 0.441 | 0.227 | 0.348 | 9.0 | 14 |
| Inhalation | 15.3 | 14.7 | 265 | 267 | 17.3 | 18.2 | 0.329 | 0.395 | 0.534 | 0.688 | UD | UD |
| Oral | 10 | 10 | 13.7 | 8.78 | 1.37 | 0.878 | UD | UD | 2.87 | 3.69 | UD | UD |
| % F Inhalation | | | | | 15.3% | 24.3% | | | | | | |
| % F oral | | | | | 1.2% | 1.2% | | | | | | |

^{a)}Plasma t_{1/2} values calculated: Group 4 (IV), 0.083 to 1.0 hr. Group 6 (Inh), 0.333 to 3.333 hr

### II. Pre-clinical Rodent Studies

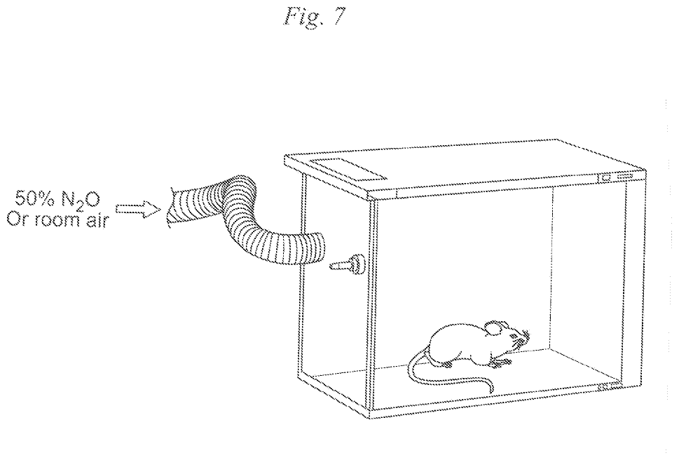

General experimental setup. Adult male laboratory mice (C57Bl6/J) will be systemically dosed with a bolus of N,N-dimethyltryptamine (DMT, 1, 3 or 10 mg/kg subcutaneous, s.c.) as fumarate salt or vehicle (saline) as control and immediately placed into a familiar transparent air-tight plexiglass anesthetic induction chamber which is linked to a controlled airflow allowing the inhalational administration of medical grade nitrous oxide, N₂O (50%) in room air or oxygen, or 100% room air or oxygen in controls, at a flow rate of 4-8 l/ml for the duration of 1 hour, for example as depicted in FIG. 7. Following 1 h treatment, all mice will be returned to room air in the chambers for a further 1 h before brain tissue and blood are extracted for molecular analysis.

Dose rationale: Psychedelic compounds elicit profound effects over the serotonergic system, which could translate to long-term increased synaptic serotonin availability (see Inserra, ., De Gregorio, D. & Gobbi, G. Psychedelics in Psychiatry: Neuroplastic, Immunomodulatory, and Neurotransmitter Mechanisms. Pharmacol Rev 73, 202-277 (2021)). Preclinical studies with DMT and other psychedelic drugs show potent and region-specific modulation of serotonin release in the brain (see Kelmendi, B., Kaye, A. P., Pittenger, C. & Kwan, A. C. Psychedelics. Curr Biol 32, R63-R67 (2022)). For example, a study quantifying monoaminergic changes in the rat brain found that DMT in the form of ayahuasca increases serotonin in the hippocampus and in the amygdala (see de Castro-Neto, E. F. et al. Changes in aminoacidergic and monoaminergic neurotransmission in the hippocampus and amygdala of rats after ayahuasca ingestion. World J Biol Chem 4, 141-147 (2013)). Similarly, a study investigating the effects of ayahuasca administration found an increase in whole brain serotonin levels in female rats receiving repeated ayahuasca administration (see Colago, C. S. et al. Toxicity of ayahuasca after 28 days daily exposure and effects on monoamines and brain-derived neurotrophic factor (BDNF) in brain of Wistar rats. Metab Brain Dis 35, 739-751 (2020)). As described herein, dosages of the administered drugs can be varied depending upon the requirements of the subject and the psychedelic drug being used. The dose of the psychedelic drug administered to a subject, in this case DMT, should be sufficient to affect a beneficial therapeutic response in the subject over time.

Experiments in rats and mice describe dose ranges from 1-10 mg/kg. Preclinical studies in rats and mice indicate that a 1 mg/kg dose of DMT (intraperitoneal, i.p.) is sub-hallucinogenic (see Cameron, L. P., Benson, C. J., DeFelice, B. C., Fiehn, O. & Olson, D. E. Chronic, Intermittent Microdoses of the Psychedelic N,N-Dimethyltryptamine (DMT) Produce Positive Effects on Mood and Anxiety in Rodents. ACS Chem. Neurosci. 10, 3261-3270 (2019))—as measured by head-twitch responses (HTRs), and studies also show that HTRs are evoked in a dose-dependent manner (see Halberstadt, A. L., Chatha, M., Klein, A. K., Wallach, J. & Brandt, S. D. Correlation between the potency of hallucinogens in the mouse head-twitch response assay and their behavioral and subjective effects in other species. Neuropharmacology 167, 107933 (2020); Carbonaro, T. M. et al. The role of 5-HT_{2A}, 5-HT_{2C} and mGlu2 receptors in the behavioral effects of tryptamine hallucinogens N,N-dimethyltryptamine and N,N-diisopropyltryptamine in rats and mice. Psychopharmacology 232, 275-284 (2015)). In rats, DMT has a half-life of 5-15 min following intraperitoneal injection and is rapidly metabolized and cleared from brain, liver, and plasma within 1 h (see Sitaram, B. R., Lockett, L., Talomsin, R., Blackman, G. L. & McLeod, W. R. In vivo etabolism of 5-methoxy-N, N-dimethyltryptamine and N,N-dimethyltryptamine in the rat. Biochemical Pharmacology 36, 1509-1512 (1987)).

These studies aim to define proof-of-concept synergistic interactions of DMT and N₂O based on previous experimental studies of each molecule individually. By exposing mice to 3 different doses of DMT in the presence or absence of N₂O—which exerts NMDA receptor antagonist effects—it will be determined whether there is a synergistic, dose-response interaction between DMT and N₂O. As such, in the preclinical experiments in mice, DMT will be dosed systemically via subcutaneous (s.c.) injection, and N₂O dosed via continual inhalation.

Experimental groups (n=8/group) will be as described in Table 5.

TABLE 5

| | | | | |
|---|---|---|---|---|
| Room air | Vehicle | 1 mg/kg DMT | 3 mg/kg DMT | 10 mg/kg DMT |
| N₂O 50% | Vehicle | 1 mg/kg DMT | 3 mg/kg DMT | 10 mg/kg DMT |

Experiment 1-Effect of N₂O on the pharmacodynamic effects of DMT. The dose-dependent behavioral effects of N₂O on DMT-induced head twitch response (HTR) in mice will be determined.

Rationale: The head-twitch response (HTR) is a rapid side-to-side head movement that occurs in mice and rats after the serotonin 5-HT_{2A} receptor is activated. The HTR is widely used as a behavioral assay for 5-HT_{2A} activation and to probe for interactions between the 5-HT_{2A} receptor and other transmitter systems (see Halberstadt, A. L. & Geyer, M. A.

Characterization of the head-twitch response induced by hallucinogens in mice: detection of the behavior based on the dynamics of head movement. Psychopharmacology (Berl) 227, 10.1007/s00213-013-3006-z (2013); Canal, C. E. & Morgan, D. Head-twitch response in rodents induced by the hallucinogen 2,5-dimethoxy-4-iodoamphetamine: a comprehensive history, a re-evaluation of mechanisms, and its utility as a model. Drug Test Anal 4, 556-576 (2012)). The administration of N₂O in rats has been shown to increase serotonin turnover in the hypothalamus, decreased turnover in the frontal cortex but no changes in either hippocampus or corpus striatum (see Emmanouil, D. E., Papadopoulou-Daifoti, Z., Hagihara, P. T., Quock, D. G. & Quock, R. M. A study of the role of serotonin in the anxiolytic effect of nitrous oxide in rodents. Pharmacology Biochemistry and Behavior 84, 313-320 (2006)), indicating that although N₂O does not directly bind to 5-HT receptors, it can alter the metabolism and release of serotonin in key brain areas involved in arousal and cognition. It is currently untested as to whether N₂O by itself evokes HTR in mice, however as N₂O does not have documented affinity for 5-HT_{2A}R it is unlikely, and this will be tested in the N₂O vehicle group. However, other NMDA antagonist molecules, such as MK-801, have been shown to increase prefrontal cortical levels of glutamate and enhance the effects of the 5-HT_{2A} agonist DOI, shown by increased HTRs and locomotor activity in rats elicited by doses of DOT (0.313-1.25 mg/kg i.p.)(see Zhang, C. & Marek, G. J. AMPA receptor involvement in 5-hydroxytryptamine2A receptor-mediated pre-frontal cortical excitatory synaptic currents and DOI-induced head shakes. Progress in Neuro-Psychopharmacology and Biological Psychiatry 32, 62-71 (2008)).

By exposing mice to 3 different doses of the 5-HT_{2A} agonist DMT in the presence or absence of N₂O, a weak NMDA receptor antagonist, it will be determined whether there is a synergistic, dose-response interaction between DMT and N₂O through the number of HTRs elicited by each animal, allowing the definition of pharmacodynamic interactions between each substance. Analyses will be conducted that examine the between groups factor of carrier gas (N₂O/control) and dose of DMT.

Method: Each chamber has a high-speed video camera set up to record the 60 mi drug session, allowing an independent observer to quantify the number of HTR behaviors performed by the mice in each drug condition.

A synergistic effect of N₂O and DMT on 5-HT_{2A}R activation would be demonstrated by an increase in HTR compared to the same dose of DMT in without N₂O. Calculation of the dose-response curves comparing total HTR evoked by DMT doses in N₂O vs. without N₂O will establish whether DMT potency is increased by co-administration of N₂O, demonstrated by a leftward shift of the dose-response curve. By plotting the cumulative number of HTR per 1 mi in each drug dose condition, the area under the curve will be calculated to establish dose-dependent changes.

Clinical implications. Left-shifted dose-response curve and/or increased HTR responses will demonstrate increased potency of DMT in combination with N₂O, and thus lower therapeutic doses may be efficacious in a clinical setting.

Experiment 2-Effect of N₂O+DMT on the brain and blood neuroplasticity biomarkers. The effects of DMT plus N₂O on the expression of blood and brain biomarkers associated with neuroplasticity will be determined.

Rationale: In a preclinical setting, DMT and N₂O have been shown to increase molecular markers of neuroplasticity in the brain. Kohtala et al (2019) demonstrated significant increases in mRNA levels of arc, bdnf, synapsin-1 homer-1, and cfos in the medial prefrontal cortex after administration of N₂O (50%) to mice for 1 h followed by a 1 h washout period (see Kohtala, S. et al. Cortical Excitability and Activation of TrkB Signaling During Rebound Slow Oscillations Are Critical for Rapid Antidepressant Responses. Mol Neurobiol 56, 4163-4174 (2019)). Moreover, repeated exposure to N₂O promoted the formation of new neurons in the brain (neurogenesis) in rats (see Chamaa, F. et al. Nitrous Oxide Induces Prominent Cell Proliferation in Adult Rat Hippocampal Dentate Gyrus. Frontiers in Cellular Neuroscience 12, 135 (2018)), a neuronal process shown to be augmented by BDNF (see Henry, R. A., Hughes, S. M. & Connor, B. AAV-mediated delivery of BDNF augments neurogenesis in the normal and quinolinic acid-lesioned adult rat brain. European Journal of Neuroscience 25, 3513-3525 (2007)). The 5-HT_{2A} agonist DOI operates through the release of VEF, and has been shown to induce profound regeneration of the liver through activation of VEGF pathways (see Furrer, K. et al. Serotonin reverts age-related capillarization and failure of regeneration in the liver through a VEGF-dependent pathway. Proc Natl Acad Sci USA 108, 2945-2950 (2011)). Similarly, treatment with DMT increased cortical bdnf mRNA and serum BDNF protein in a rat model of stroke (see Nardai, S. et al. N,N-dimethyltryptamine reduces infarct size and improves functional recovery following transient focal brain ischemia in rats. Experimental Neurology 327, 113245 (2020)), increased PFC dendritic spine density in rats (see Ly, C. et al. Psychedelics Promote Structural and Functional Neural Plasticity. Cell Rep 23, 3170-3182 (2018)), and increased neurogenesis the hippocampus in mice (see Morales-Garcia, J. A. et al. N,N-dimethyltryptamine compound found in the hallucinogenic tea ayahuasca, regulates adult neurogenesis in vitro and in vivo. Transl Psychiatry 10, 1-14 (2020)).

Method: Following the 60 min DMT/N₂O treatment session, mice will be left in the plexiglass chambers with continual flow of room air for a 60 mi washout. Mice will then be sacrificed by decapitation, cardiac blood samples taken and brains extracted. The frontal cortex, hippocampus, striatum and olfactory bundle will be microdissected from brains and snap frozen in liquid nitrogen to allow protein and gene expression analysis. Blood will be centrifuged to obtain plasma for analysis. A panel of proteomic and genetic biomarkers selected based upon their relative brain-specificities and potentials to reflect distinct neurobiological alterations will be run. Analyses will be conducted that examine the between groups factor of carrier gas (N₂O/control) and dose of DMT.

Gene expression analysis—mRNA of molecular targets involved in the regulation of synaptic plasticity, synaptogenesis and glutamate signaling will be quantified by real-time PCR. Genes of interest will include: bdnf, vegf, synapsin-1, Dlg4 (PSD-95), mtorc1, creb1, Grm1, homer1. Data will be analyzed using the 2^{-ΔΔCT} method (see Livak, K. I & Schmittgen, T. D. Analysis of Relative Gene Expression Data Using Real-Time Quantitative PCR and the 2−ΔΔCT Method. Methods 25, 402-408 (2001)), and fold-expression presented over the normalized mean of the control/vehicle group.

Protein analysis—Western blots will be performed to quantify protein levels of phosphorylated biomarkers: p-TrkB, p-MAPK/p-ERK, and glycogen synthase kinase 3β (p-GSK3β). Plasma will be analyzed by ELISA to determine BDNF and VEGF levels.

A synergistic effect of N₂O and DMT on neuroplasticity would be demonstrated by a significant increase in levels of the specified neuroplasticity biomarkers compared to the same dose of DMT when mice are not exposed to N₂O. As both N₂O and DMT have been demonstrated to increase neuroplasticity biomarkers and activity related immediate early genes (see Kohtala, S. et al. Cortical Excitability and Activation of TrkB Signaling During Rebound Slow Oscillations Are Critical for Rapid Antidepressant Responses. Mol Neurobiol 56, 4163-4174 (2019); Nardai, S. et al. N,N-dimethyltryptamine reduces infarct size and improves functional recovery following transient focal brain ischemia in rats. Experimental Neurology 327, 113245 (2020); and Ly, C. et al. Psychedelics Promote Structural and Functional Neural Plasticity. Cell Rep 23, 3170-3182 (2018)), it is highly feasible that synergistic effects will be seen in the N₂O and DMT, or increased levels of these markers with the lower DMT doses when N₂O is present.

Clinical implication: This experiment will demonstrate whether the addition of N₂ as a carrier gas for inhalational DMT will synergistically enhance markers of neuroplasticity, and potentially allow a lower therapeutic dose of DMT to be used in the clinic.

Experiment 3—Effect of N₂O+DMT on stress reactivity. The effects of DMT plus N₂O on expression of endocrine biomarkers of stress and HPA-axis activation will be determined.

Rationale: Psychedelics may produce challenging experiences, often characterized as “bad trips” (see Barrett, F. S., Bradstreet, M. P., Leoutsakos, J.-M. S., Johnson, M. W. & Griffiths, R. R. The Challenging Experience Questionnaire: Characterization of challenging experiences with psilocybin mushrooms. J Psychopharmacol 30, 1279-1295 (2016); Carbonaro, T. M. et al. Survey study of challenging experiences after ingesting psilocybin mushrooms: Acute and enduring positive and negative consequences. J Psychopharmacol 30, 1268-1278 (2016)). Although bad trips are unpleasant, research suggests that challenging experiences may be key to the potential beneficial effects of psychedelic substances (see Barrett, F. S., Bradstreet, M. P., Leoutsakos, J.-M. S., Johnson, M. W. & Griffiths, R. R. The Challenging Experience Questionnaire: Characterization of challenging experiences with psilocybin mushrooms. J Psychopharmacol 30, 1279-1295 (2016); Gashi, L., Sandberg, S. & Pedersen, W. Making “bad trips” good: How users of psychedelics narratively transform challenging trips into valuable experiences. International Journal of Drug Policy 87, 102997 (2021); and Carhart-Harris, R. L. et al. Psilocybin with psychological support for treatment-resistant depression: an open-label feasibility study. The Lancet Psychiatry 3, 619-627 (2016)). Griffiths et al. (2006) found that high doses of psilocybin created fear in 30% of the study participants, yet 80% of them reported improvement in well-being (see Griffiths, R. R., Richards, W. A., McCann, U. & Jesse, R. Psilocybin can occasion mystical-type experiences having substantial and sustained personal meaning and spiritual significance. Psychopharmacology 187, 268-283 (2006)). Responses to psychedelic drug are highly dependent to the user's mindset, mood and their expectations (see Studerus, E., Gamma, A., Kometer, M. & Vollenweider, F. X. Prediction of Psilocybin Response in Healthy Volunteers. PLOS ONE 7, e30800 (2012)). Studies indicate that the “set and setting” of substance use influence an individual's reaction how people respond expectations (see Studerus, E., Gamma, A., Kometer, M. & Vollenweider, F. X. Prediction of Psilocybin Response in Healthy Volunteers. PLOS ONE 7, e30800 (2012)) and a cornerstone of psychedelic-assisted psychotherapy is the promotion of a calming, safe environment and psychological support.

In clinical settings, N₂O is widely used as a sedative and as a carrier gas for other anesthetic agents (such as volatile anesthetics halothane, isoflurane, desflurane, and sevoflurane), and at low dosage in humans and animals, N₂O relieves anxiety (see Emmanouil, D. E., Papadopoulou-Daifoti, Z., Hagihara, P. T., Quock, D. G. & Quock, R. M. A study of the role of serotonin in the anxiolytic effect of nitrous oxide in rodents. Pharmacology Biochemistry and Behavior 84, 313-320 (2006); Sundin, R. H. et al. Anxiolytic effects of low dosage nitrous oxide-oxygen mixtures administered continuously in apprehensive subjects. South Med J 74, 1489-1492 (1981); Zacny, J. P., Hurst, R. J., Graham, L. & Janiszewski, D. J. Preoperative dental anxiety and mood changes during nitrous oxide inhalation. J Am Dent Assoc 133, 82-88 (2002); and Li, L. et al. Comparison of analgesic and anxiolytic effects of nitrous oxide in burn wound treatment: A single-blind prospective randomized controlled trial. Medicine 98, e18188 (2019)). Furthermore, the rapid onset of anxiolytic action of N₂O makes it useful for relieving anxiety prior to medical procedures. Preclinical studies indicate that N₂O can activate the endogenous inhibitory input to the hypothalamus-pituitary-adrenal (HPA) axis (see Himukashi, S., Takeshima, H., Koyanagi, S., Shichino, T. & Fukuda, K. The Involvement of the Nociceptin Receptor in the Antinociceptive Action of Nitrous Oxide. Anesthesia & Analgesia 103, 738-741 (2006)), and in people N₂O elicited significant decrease in serum cortisol levels, blood pressure and pulse rate in individuals undergoing dental procedures, which was associated with decreased subjective reports of stress (see Sandhu, G. et al. Comparative evaluation of stress levels before, during, and after periodontal surgical procedures with and without nitrous oxide-oxygen inhalation sedation. J Indian Soc Periodontol 21, 21-26 (2017)). Administration of DMT in the form of ayahuasca and 5-MeO-DMT have been associated with increased salivary cortisol in people (see Galvão, A. C. de M. et al. Cortisol Modulation by Ayahuasca in Patients With Treatment Resistant Depression and Healthy Controls. Front Psychiatry 9, 185 (2018); Uthaug, M. V. et al. Prospective examination of synthetic 5-methoxy-N,N-dimethyltryptamine inhalation: effects on salivary IL-6, cortisol levels, affect, and non-judgment. Psychopharmacology 237, 773-785 (2020)), and anxiety-like behavior in rats (see Cameron, L. P., Benson, C. J., Dunlap, L. E. & Olson, D. E. Effects of N,N-dimethyltryptamine (DMT) on rat behaviors relevant to anxiety and depression. ACS chemical neuroscience 9, 1582 (2018)). Therefore, DMT plus N₂O will be co-administered to determine whether there is a decrease of stress biomarkers in blood.

Method: As described previously, mice will be left in the plexiglass chambers with continual flow of room air for a 60 mi washout after the 60 min DMT/N₂O treatment session. Mice will then be sacrificed by decapitation, cardiac blood samples taken and brains extracted. A panel of proteomic biomarkers selected based upon their potentials to reflect distinct biological alterations will be run.

Endocrine biomarkers—Acute stress stimulates the release of adrenocorticotropic hormone (ACTH) from the anterior pituitary, which acts on the adrenal cortex to induce release of glucocorticoids including corticosterone and epinephrine. In the hypothalamus, β-endorphin neurons innervate corticotropin-releasing hormone (CRH) neurons and inhibit CRH release. β-endorphin plays an important physiological role in analgesia, regulation and release of pituitary hormones, amelioration of anxiety, appetitive behavior, temperature regulation, and other visceral functions. Plasma ACTH, corticosterone, β-endorphin and epinephrine concentrations will be measured using commercially available ELISA kits to examine the difference of stress hormonal response in each experimental group. Analyses will be conducted that examine the between groups factor of carrier gas (N₂O/control) and dose of DMT.

As N₂O has anxiolytic properties it is feasible that stress-associated biomarkers will be reduced following administration of DMT in the N₂O groups compared to the controls.

Clinical implication: This experiment will demonstrate whether the addition of N₂O as a carrier gas for inhalational DMT will alleviate anxiety and apprehension in patients, creating a supportive setting for effective psychedelic-assisted psychotherapy.

Experiment 4—Examine the effect of N₂O DMT on neural oscillations. The synergistic effects of DMT plus N₂O on neural oscillations will be determined using local field potential recordings in awake mice.

Rationale: Neural oscillations are rhythmic or repetitive patterns of neural activity generated spontaneously in different states of consciousness, and in response to stimuli. rats, 5-MeO-DMT increased pyramidal firing rate and low frequency oscillations in the medial prefrontal cortex using local field potential recordings (see Riga, M. S., Soria, G., Tudela, R., Artigas, F. & Celada, P. The natural hallucinogen 5-MeO-DMT, component of Ayahuasca, disrupts cortical function in rats: reversal by antipsychotic drugs. International Journal of Neuropsychopharmacology 17, 1269-1282 (2014)). In mice, N₂O exposure increased cortical slow wave delta (1-4 Hz) and theta (4-7 Hz) oscillations upon N₂O withdrawal, which is when pleiotropic changes in neuroplasticity is thought to occur (see Kohtala, S. & Rantamäki, T. Rapid-acting antidepressants and the regulation of TrkB neurotrophic signalling-Insights from ketamine, nitrous oxide, seizures and anaesthesia. Basic & Clinical Pharmacology & Toxicology 129, 95-103 (2021)). Rebound increases in delta oscillations are observed after the discontinuation of N₂O treatment which coincides with the upregulation of neuroplasticity biomarkers, including the phosphorylation of BDNF receptor TrkB and GSK3β (glycogen synthase kinase 3p). Moreover, NMDA receptor antagonism with ketamine in rats was shown to significantly increase tissue oxygen in both the striatum and the hippocampus, along with significant decreases in delta and alpha power along with increases in theta and gamma power in the hippocampus (see Kealy, J., Commins, S. & Lowry, J. P. The effect of NMDA-R antagonism on simultaneously acquired local field potentials and tissue oxygen levels in the brains of freely-moving rats. Neuropharmacology 116, 343-350 (2017)).

In people, a high dose of N₂O is associated with large amplitude slow-delta oscillations, potentially due to blockade of NMDA glutamate projections from the brainstem to the thalamus and cortex (see Pavone, K. J. et al. Nitrous oxide-induced slow and delta oscillations. Clin Neurophysiol 127, 556-564 (2016)). Similarly, DMT administration alters neural oscillations across different frequency bands in both rodents (see Morley, B. & Bradley, R. Spectral analysis of mouse EEG after the administration of N,N-dimethyltryptanine. Biological psychiatry 12, 757-69 (1978))³⁹ and humans (see Timmermann, C. et al. Neural correlates of the DMT experience assessed with multivariate EEG. Sci Rep 9, 16324 (2019); Tagliazucchi, E. et al. Baseline Power of Theta Oscillations Predicts Mystical-Type Experiences Induced by DMT in a Natural Setting. Frontiers in Psychiatry 12, 1922 (2021); and Pallavicini, C. et al. Neural and subjective effects of inhaled N,N-dimethyltryptamine in natural settings. J Psychopharmacol 35, 406-420 (2021)), and has generally shown to decrease spectral power in alpha and beta frequency bands (see Timmermann, C. et al. Neural correlates of the DMT experience assessed with multivariate EEG. Sci Rep 9, 16324 (2019); Tagliazucchi, E. et al. Baseline Power of Theta Oscillations Predicts Mystical-Type Experiences Induced by DMT in a Natural Setting. Frontiers in Psychiatry 12, 1922 (2021); and Pallavicini, C. et al. Neural and subjective effects of inhaled N,N-dimethyltryptamine in natural settings. J Psychopharmacol 35, 406-420 (2021)), increases in spontaneous signal diversity and the emergence of delta and theta oscillations are reported during peak effects (see Timmermann, C. et al. Neural correlates of the DMT experience assessed with multivariate EEG. Sci Rep 9, 16324 (2019)).

Method: Adult male mice will be surgically implanted with wireless amperometric sensors in the medial prefrontal cortex, somatosensory cortex, striatum and hippocampus, allowing the simultaneous measurement of electrical activity and tissue oxygen in the brains of freely-moving mice. After a recovery period, mice will be habituated to the anesthesia chambers and baseline recordings conducted. Power spectrum analysis in each bandwidth (delta—1-4 Hz; theta=4-7 Hz; alpha=7-12 Hz; beta=12-30 Hz; gamma low=30-60 Hz; gamma high=60-100 Hz) will be computed. The total recording time will be 120 min, accounting for 60 mins of N₂O treatment followed by 60 mins of room air washout.

Based on previous data detailing separate effects of DMT and N₂O, alterations in LFP power spectra at both low and high frequency oscillations are possible. NMDA receptor antagonism with ketamine caused significant increases in tissue oxygenation in both the striatum and the hippocampus (see Kealy, J., Commins, S. & Lowry, J. P. The effect of NMDA-R antagonism on simultaneously acquired local field potentials and tissue oxygen levels in the brains of freely-moving rats. Neuropharmacology 116, 343-350 (2017)), as such a similar effect with N₂O is feasible. Following removal of N₂O and return to room air, it will be determined whether a refractory increase in low frequency oscillations (delta, theta) across the 60 mi washout in this condition is seen (see Kohtala, S. et al. Cortical Excitability and Activation of TrkB Signaling During Rebound Slow Oscillations Are Critical for Rapid Antidepressant Responses. Mol Neurobiol 56, 4163-4174 (2019)), but this increase will not be observed in the DMT+control gas condition.

Clinical implication: This experiment will demonstrate whether the addition of N₂O as a carrier gas for inhalational DMT will alter the spectra of low frequency neural oscillations. The addition of N₂O is proposed to result in an extended window of neuroplasticity upregulation following cessation of N₂O that correlates with increased delta oscillation power (see Kohtala, S. et al. Cortical Excitability and Activation of TrkB Signaling During Rebound Slow Oscillations Are Critical for Rapid Antidepressant Responses. Mol Neurobiol 56, 4163-4174 (2019); Kohtala, S. & Rantamäki, T. Rapid-acting antidepressants and the regulation of TrkB neurotrophic signalling-Insights from ketamine, nitrous oxide, seizures and anaesthesia. Basic & Clinical Pharmacology & Toxicology 129, 95-103 (2021)). This will demonstrate whether the use of N₂O as a carrier gas can enhance the therapeutic efficacy of DMT.

### III. Human Studies

General experimental design. The proposed studies aim to define proof-of-concept synergistic interactions of inhalational DMT fumarate with N₂O, or IV DMT fumarate administered as a bolus over 30 seconds. Healthy adult participants will be exposed to either inhalational DMT in 20-25% N₂O in oxygen as the carrier gas, or inhalational DMT in oxygen alone as the carrier gas in a blinded manner, or IV DMT while inhaling 20-25% N₂O in oxygen, or oxygen. A recent clinical trial showed that 25% N₂O inhaled across 60 mins was well tolerated and associated with an improved safety profile of unwanted effects in comparison to a therapeutic concentration of 50% N₂O in the setting of treatment resistant depression (see Nagele, P. et al. A phase 2 trial of inhaled nitrous oxide for treatment-resistant major depression. Science Translational Medicine (2021)). The inhalational delivery device for delivery of a combination of N₂O and a psychedelic drug—in this exemplar DMT—to humans is described herein. Briefly, the inhalation delivery device comprises an inhalation outlet portal for administration of the combination of N₂O and the psychedelic drug to the patient; a container configured to deliver N₂O gas to the inhalation outlet portal; and a device configured to generate and deliver an aerosol comprising the psychedelic drug to the inhalation outlet portal. The DMT (fumarate) will be prepared as an aqueous solution through dissolution in water or buffer (e.g., citric acid buffer), or as an aqueous emulsion by dispersing the liquid psychedelic drug, in this case DMT, or derivative thereof in water with viscous material.

Dose of DMT. Doses of DMT between e.g., 0.01-10 mg/kg will be utilized depending on the infusion procedure. Previous literature in human participants using an IV bolus of DMT (as fumarate salt) in doses between 0.05-0.4 mg/kg demonstrated that the psycho-biological effects occur immediately after administration, with a peak at 120 seconds, and resolve by 30 minutes (see Strassman, R. J., Qualls, C. R., Uhlenhuth, E. H. & Kellner, R. Dose-Response Study of N,N-Dimethyltryptamine in Humans: II. Subjective Effects and Preliminary Results of a New Rating Scale. Archives of General Psychiatry 51, 98-108 (1994); Strassman, R. J. & Qualls, C. R. Dose-Response Study of N,N-Dimethyltryptamine in Humans: I. Neuroendocrine, Autonomic, and Cardiovascular Effects. Archives of General Psychiatry 51, 85-97 (1994); and Gallimore, A. R. & Strassman, R. J. A Model for the Application of Target-Controlled Intravenous Infusion for a Prolonged Immersive DMT Psychedelic Experience. Frontiers in Pharmacology 7, (2016)). When administered intravenously, DMT reaches peak plasma concentrations in approximately 2 minutes and the half-life of DMT is around 15 minutes (see Carbonaro, T. M. & Gatch, M. B. Neuropharmacology of N,N-Dimethyltryptamine. Brain Res Bull 126, 74-88 (2016)). Commonly used doses for vaporized or inhaled free-base DMT are 40-50 mg (0.57-0.71 mg/kg in a 70 kg human)(see Barker, S. A. N, N-Dimethyltryptamine (DMT), an Endogenous Hallucinogen: Past, Present, and Future Research to Determine Its Role and Function. Frontiers in Neuroscience 12, (2018)). The onset of vaporized DMT is rapid, similar to that of IV administration, but lasts less than 30 min (see Barker, S. A. N, N-Dimethyltryptamine (DMT), an Endogenous Hallucinogen: Past, Present, and Future Research to Determine Its Role and Function. Frontiers in Neuroscience 12, (2018)). Therefore, the full duration of the study session with exposure to DMT+N₂O will be 1 h, with a further assessment at 2 h and 24 h following administration.

Two weeks prior to the beginning of the experimental sessions, participants will be requested to abstain from any medication or illicit drug until the completion of the study. Participants will also be instructed to abstain from alcohol, tobacco, and caffeinated drinks 24 h prior to the experimental day. Participants will arrive in the laboratory in the morning under fasting conditions. The experimental sessions will be undertaken in a quiet and dimly lit room with the participants seated in a reclining chair or bed. Participants will have an eye mask and two trained facilitators will be present throughout the session.

The general experimental session timeline is as follows:

Pre-study: Baseline measurements of heart rate, body temperature and blood pressure will be made. An IV cannula will be inserted into a forearm vein for blood sampling and to allow administration of DMT as a bolus in the IV condition. Participants will be allowed to relax for 30 min before the drug session.
Study session: Participants will receive administration of either 20-25% N₂O in oxygen, or oxygen alone, for 10 minutes prior to the administration of a high, medium or low dose of inhalational or IV DMT (0.4, 0.2, 0.1 mg/kg) over the course of 30 sec-1 minute. The method of delivering a psychedelic drug to the CNS via inhalation can increase bioavailability, therefore the dose range of DMT tested is from sub-psychedelic (0.1 mg/kg) to a putative “high” dose (0.4 mg/kg), these doses have been previously characterized via IV administration in healthy volunteers (see Strassman, R. J., Qualls, C. R., Uhlenhuth, E H. & Kellner, R. Dose-Response Study of N,N-Dimethyltryptamine in Humans: II. Subjective Effects and Preliminary Results of a New Rating Scale. Archives of General Psychiatry 51, 98-108 (1994); Strassman, R. J. & Qualls, C. R. Dose-Response Study of N,N-Dimethyltryptarine in Humans: I. Neuroendocrine, Autonomic, and Cardiovascular Effects. Archives of General Psychiatry 51, 85-97 (1994)), whereas a higher dose of 0.7 mg/kg of DMT has been reported to be administered via intramuscular injection (see Kaplan, J. et al. Blood and urine levels of N,N-dimethyltryptamine following administration of psychoactive dosages to human subjects. Psychopharmacologia 38, 239-245 (1974)).

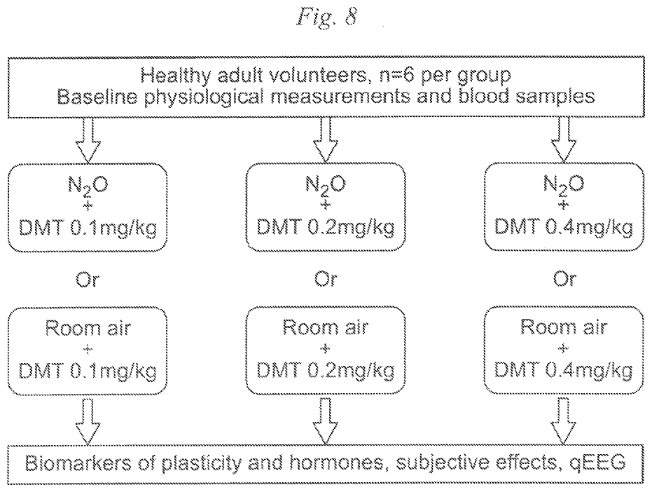

Participants will be exposed to one of 3 different doses of inhalational DMT or IV DMT in the presence or absence of N₂O—a weak NMDA receptor antagonist—with the aim of demonstrating a synergistic, dose-response interaction between DMT and N₂O. The study design is depicted in FIG. 8. The design allows for carryover effects to be excluded, a range of doses to be evaluated to identify most efficacious doses, and to more than one administration to be evaluated to identify greatest efficacy.

N₂O will be administered for 10 minutes prior to the administration of DMT. At a low dosage in humans and animals (i.e. <50%), N₂O relieves anxiety and promotes relaxation and calnmess (see Emmanouil, D. E., Papadopoulou-Daifoti, Z., Hagihara, P. T., Quock, D. G. & Quock, R. M. A study of the role of serotonin in the anxiolytic effect of nitrous oxide in rodents. Pharmacology Biochemistry and Behavior 84, 313-320 (2006); Sundin, R. H. et al. Anxiolytic effects of low dosage nitrous oxide-oxygen mixtures administered continuously in apprehensive subjects. South Med J 74, 1489-1492 (1981); Zacny, J. P., Hurst, R. J., Graham, L & Janiszewski, D. J. Preoperative dental anxiety and mood changes during nitrous oxide inhalation. J Am Dent Assoc 133, 82-88 (2002); Li, L. et al. Comparison of analgesic and anxiolytic effects of nitrous oxide in burn wound treatment: A single-blind prospective randomized controlled trial. Medicine 98, e18188 (2019)) with a rapid onset (see Sandhu, G. et al. Comparative evaluation of stress levels before, during, and after periodontal surgical procedures with and without nitrous oxide-oxygen inhalation sedation. J Indian Soc Periodontol 21, 21-26 (2017)). Participants will receive inhalational N₂O or oxygen for 60 mins in total, and then be returned to room air. Noninvasive blood pressure, percutaneous arterial blood oxygen saturation (SpO2), and the pulse rate will be periodically measured throughout the study session. Two experimenters will be present throughout the study session.

Experiment 5—Quantification of the pharmacokinetic and psychedelic effects of DMT+N₂O in healthy human participants. The synergistic effects of DMT plus N₂O on the psychedelic experience in healthy human participants will be determined as measured by reports of subjective effects.

Rationale: Previous studies have shown that 0.2 and 0.4 mg/kg DMT (IV) evoke nearly instantaneous onset of visual hallucinatory phenomena, bodily dissociation, and extreme shifts in mood, whereas 0.1 mg/kg is not hallucinogenic, but results in emotional and somesthetic effects (see Strassman, R. J., Qualls, C. R., Uhlenhuth, E. H. & Kellner, R. Dose-Response Study of N,N-Dimethyltryptamine in Humans: IL Subjective Effects and Preliminary Results of a New Rating Scale. Archives of General Psychiatry 51, 98-108 (1994)).

At a low dosage in humans and animals, N₂O relieves anxiety and can promote feelings of euphoria, relaxation and calmness (see Emmanouil, D. E., Papadopoulou-Daifoti, Z., Hagihara, P. T., Quock, D. G. & Quock, R. M. A study of the role of serotonin in the anxiolytic effect of nitrous oxide in rodents. Pharmacology Biochemistry and Behavior 84, 313-320 (2006); Sundin, R. H. et al. Anxiolytic effects of low dosage nitrous oxide-oxygen mixtures administered continuously in apprehensive subjects. South Med J 74, 1489-1492 (1981); Zacny, J. P., Hurst, R. J., Graham, L. & Janiszewski, D. J. Preoperative dental anxiety and mood changes during nitrous oxide inhalation. J Am Dent Assoc 133, 82-88 (2002); and Li, L. et al. Comparison of analgesic and anxiolytic effects of nitrous oxide in burn wound treatment: A single-blind prospective randomized controlled trial. Medicine 98, e18188 (2019)). Furthermore, the rapid onset of anxiolytic action of N₂O makes it suited for relieving anxiety and apprehension prior to medical procedures (see Sandhu, G. et al. Comparative evaluation of stress levels before, during, and after periodontal surgical procedures with and without nitrous oxide-oxygen inhalation sedation. J Indian Soc Periodontol 21, 21-26 (2017)). As responses to psychedelic drugs are highly dependent to the user's mindset, mood and expectations (see Studerus, E., Gamma, A., Kometer, M. & Vollenweider, F. X. Prediction of Psilocybin Response in Healthy Volunteers. PLOS ONE 7, e30800 (2012)), the addition of N₂O to the DMT administration protocol can aid with the alleviation of pre-treatment anxiety, and reduce the likelihood of a “bad trip”.

Method: Baseline blood samples for measuring blood DMT concentrations will be drawn 30 minutes before 25% N₂O or oxygen administration, and after 2, 5, 10,15, 30 and 60 minutes after DMT administration.

Following the administration of DMT with/without N₂O, participants will undergo clinical interviews where participants will be requested to answer a series of questionnaires to assess efficacy. These assessments can include the Mystical Experience Questionnaire-30 Item (MEQ-30) (see Maclean, K. A., Leoutsakos, J.-M. S., Johnson, M. W. & Griffiths, R. R. Factor Analysis of the Mystical Experience Questionnaire: A Study of Experiences Occasioned by the Hallucinogen Psilocybin. J Sci Study Relig 51, 721-737 (2012)), 5-Dimensional Altered States of Consciousness Rating Scale (5D-ASC) (see Dittrich, A. The Standardized Psychometric Assessment of Altered States of Consciousness (ASCs) in Humans. Pharmacopsychiatry 31, 80-84 (1998)), and the Hallucinogen Rating Scale (HRS) (see Strassman, R. J., Qualls, C. R., Uhlenhuth, E. H. & Kellner, R. Dose-Response Study of N,N-Dimethyltryptamine in Humans: I. Subjective Effects and Preliminary Results of a New Rating Scale. Archives of General Psychiatry 51, 98-108 (1994)) to quantify different aspects of psychedelic-induced subjective effects. Participants will also answer the Challenging Experience Questionnaire (CEQ) (see Barrett, F. S., Bradstreet, M. P., Leoutsakos, J.-M. S., Johnson, M. W. & Griffiths, R. R. The Challenging Experience Questionnaire: Characterization of challenging experiences with psilocybin mushrooms. J Psychopharmacol 30, 1279-1295 (2016)) to measure any negative experiences, as well as general clinician-administered visual analogue scales. Analyses will be conducted that examine the between groups factor of carrier gas (N₂O or oxygen) and dose of DMT.

If N₂O changes the pharmacokinetics of DMT a greater concentration of DMT in blood reached faster or delayed clearance, or a peak experience described with a lower dose of DMT, may be observed.

It is believed that greater scores will be seen in the MEQ-30, 5D-ASC and HRS in the DMT plus N₂O groups, particularly in measures of intensity, with the potential for a decreased “break point” for hallucinations in the low dose DMT (0.1 mg/kg) in the N₂O group. It is believed that lower scores will be seen on the CEQ in the DMT plus N₂O groups, particularly in ratings of fear and physical distress.

Clinical implication: This experiment will demonstrate whether the N₂O administration will change the pharmacokinetic efficacy of DMT and increase subjective effects of DMT at lower doses, which can lead to lower therapeutic doses in the clinical setting. Furthermore, reducing the risk of an adverse experience will make patients more receptive to repeated therapeutic sessions, and increase the efficacy of therapy.

Experiment 6—Effect of DMT+N₂O on blood biomarkers in healthy human participants. The synergistic effects of DMT plus N₂O on expression of neurotrophic (BDNF, VEGF) and endocrine markers (corticotropin, beta-endorphin, prolactin, growth hormone (GH), and cortisol) in blood will be determined.

Rationale: In clinical settings, inhalational sedation using N₂O reduces a patient's psychological stress and apprehension. Physiologically, acute stress activates the hypothalamic-pituitary-adrenal (HPA) axis resulting in a sequence of hormonal changes to activate the sympathetic nervous system, including the release of corticotropin, epinephrine and cortisol. Furthermore, the administration of IV DMT results in the dose-dependent increase in growth hormone (GIH), prolactin, β-endorphin, corticotropin, and cortisol levels measured in blood (see Strassman, R. J. & Qualls, C. R. Dose-Response Study of N,N-Dimethyltryptamine in Humans: I. Neuroendocrine, Autonomic, and Cardiovascular Effects. Archives of General Psychiatry 51, 85-97 (1994)).

Endogenous neurotrophic molecules are involved in the regulation of brain plasticity. In humans, ayahuasca ingestion has been shown to increase levels of serum BDNF from baseline when measured 48 h after dosing healthy volunteers and subjects with treatment resistant depression (see Almeida, R. N. de et al. Modulation of Serum Brain-Derived Neurotrophic Factor by a Single Dose of Ayahuasca: Observation From a Randomized Controlled Trial. Frontiers in Psychology 10, 1234 (2019)). As described previously, DMT induced elevation of the cortical BDNF mRNA expression and serum BDNF protein concentration following focal brain ischemia (stroke) in rats (see Nardai, S. et al. N,N-dimethyltryptamine reduces infarct size and improves functional recovery following transient focal brain ischemia in rats. Experimental Neurology 327, 113245 (2020)), and N₂O (50%) exposure in mice for 30 min-2h increased BDNF/BDNF IV mRNA expression from samples of the prefrontal cortex. Vascular endothelial growth factor (VEGF) is an angiogenic and neurogenic factor, which has been shown to elicit antidepressant-like effects in response to different external stimuli. The 5-HT_{2A} agonist DOI can also stimulate the release of VEGF, and activation of VEGF pathways is involved in DOI-induced regeneration of liver cells (see Furrer, K. et al. Serotonin reverts age-related capillarization and failure of regeneration in the liver through a VEGF-dependent pathway. Proc Natl Acad Sci USA 108, 2945-2950 (2011)), therefore VEGF is likely to also be increased in the blood following DMT administration.

Method: A panel of proteomic biomarkers will be run selected based upon their relative brain-specificities and potentials to reflect distinct neurobiological and endocrine alterations.

Baseline blood samples will be drawn 30 minutes before N₂O or oxygen administration, and after 8 mins of N₂O or oxygen administration for endocrine markers of HPA axis activation: corticotropin, β-endorphin, prolactin, GH and cortisol levels. Further blood samples will be drawn, and vital signs measured 2, 15, 60 and 120 minutes after DMT administration.

For blood biomarkers of brain plasticity—VEGF and BDNF, a baseline blood sample will be taken at 30 mins before the drug session, and at 60 min, 120 min and 24 h post drug administration.

Analyses will be made that examine within participants measurements from baseline and time points following drug administration, with the addition of the between groups factors of gas (N₂O/oxygen) and dose of DMT.

It is believed that significantly lower levels of stress-associated endocrine markers in the DMT plus N₂O groups will be seen, as well as dose-dependent increases in these endocrine markers. It is believed that DMT dose-dependently increased levels of VEGF and BDNF and elevated levels will be seen where N₂O is used as a carrier gas compared to the oxygen group.

Clinical implication: Responses to psychedelic drug are highly dependent to the user's mindset, mood and their expectations (see Studerus, E., Gamma, A., Kometer, M. & Vollenweider, F. X. Prediction of Psilocybin Response in Healthy Volunteers. PLOS ONE 7, e30800 (2012)). Studies indicate that the “set and setting” of substance use influence an individual's reaction how people respond and a cornerstone of psychedelic-assisted psychotherapy is the promotion of a calming, safe environment and psychological support.

This experiment will demonstrate whether the addition of N₂O as a carrier gas for inhalational DMT will reduce biomarkers of stress and HPA-axis activation in patients, creating a supportive setting for effective psychedelic-assisted psychotherapy. Moreover, this experiment will demonstrate whether the addition of N₂O will enhance blood biomarkers of neuroplasticity, and potentially allow a lower therapeutic dose of DMT to be used in the clinic.

Experiment 7—Topographic pharmaco-EEG mapping of the effects of N₂O+DMT. The synergistic effects of DMT plus N₂O on neural oscillations will be determined using topographic quantitative-electroencephalography (q-EEG) recordings to study the cerebral bioavailability and time-course of effects.

Rationale: As described in Experiment 4, neural oscillations are rhythmic or repetitive patterns of neural activity. In people, a high dose of N₂O is associated with the emergence of large amplitude slow-delta oscillations (see Pavone, K. J. et al. Nitrous oxide-induced slow and delta oscillations. Clin Neurophysiol 127, 556-564 (2016)). Furthermore, DMT (IV) administration in healthy participants decreased spectral power in alpha and beta bands (see Timmermann, C. et al. Neural correlates of the DMT experience assessed with multivariate EEG. Sci Rep 9, 16324 (2019); Tagliazucchi, E. et al. Baseline Power of Theta Oscillations Predicts Mystical-Type Experiences Induced by DMT in a Natural Setting. Frontiers in Psychiatry 12, 1922 (2021); and Pallavicini, C. et al. Neural and subjective effects of inhaled N,N-dimethyltryptamine in natural settings. J Psychopharmacol 35, 406-420 (2021)), and the emergence of low frequency delta and theta oscillations coincided with reported peak effects (see Timmermann, C. et al. Neural correlates of the DMT experience assessed with multivariate EEG. Sci Rep 9, 16324 (2019)). An increase in low frequency oscillations following the cessation of N₂O that coincided with an increase in neuroplasticity biomarkers in rodents (see Kohtala, S. et al. Cortical Excitability and Activation of TrkB Signaling During Rebound Slow Oscillations Are Critical for Rapid Antidepressant Responses. Mol Neurobiol 56, 4163-4174 (2019)), therefore it is proposed that the addition of N₂O will be synergistic with DMT-induced neuroplasticity biomarker increases.

Method: Quantitative q-EEG recordings will be obtained at baseline and at regular intervals throughout the treatment session for a duration of 2 hours. Q-EEG recordings will be obtained through electrodes placed on the scalp according to the international 10/20 system on the following locations: Fp1, Fp2, F7, F3, Fz, F4, F8, T3, C3, Cz, C4, T4, T5, P3, Pz, P4, T6, O1 and O2, referenced to averaged mastoids. The spectral density curves for all artifact-free EEG epochs will be averaged for a particular experimental situation. These mean spectral curves, containing data from 1.3 to 30 Hz, will be quantified into target variables: total power, absolute and relative power across different frequency bands delta=1-4 Hz; theta=4-7 Hz; alpha=7-12 Hz; beta=12-25 Hz; gamma low=25-40 Hz; combined delta-theta, alpha and beta), the dominant frequency in Hz, absolute and relative power of the dominant frequency. Additionally, the vigilance alpha/delta-theta index will be calculated.

It is believed that significant and dose-dependent modifications of brain electrical activity will be observed following DMT administration, and that these changes will be most pronounced in the DMT+N₂O conditions. As shown in previous studies with DMT and ayahuasca (see Tirmnermann, C. et al. Neural correlates of the DMT experience assessed with multivariate EEG. Sci Rep 9, 16324 (2019); Tagliazucchi, E. et al. Baseline Power of Theta Oscillations Predicts Mystical-Type Experiences Induced by DMT in a Natural Setting. Frontiers in Psychiatry 12, 1922 (2021); Pallavicini, C. et al. Neural and subjective effects of inhaled N,N-dimethyl tamine in natural settings. J Psychopharmacol 35, 406-420 (2021)); and Riba, J. et al. Topographic pharmaco-EEG mapping of the effects of the South American psychoactive beverage ayahuasca in healthy volunteers. Br J Clin Pharmacol 53, 613-628 (2002)), it is believed that decreased absolute power in all frequency bands will be observed, most prominently in the theta band, and an increase in the alpha/delta-theta ratio.

Clinical implication: This experiment will demonstrate whether the addition of N₂O as a carrier gas for inhalational DMT, or N₂O combined with IV DMT will alter the spectra of neural oscillations, resulting in an extended window of neuroplasticity upregulation following cessation of N₂O that will lead to greater clinical efficacy.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

Accordingly, the preceding merely illustrates the principles of the methods and compositions. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the following.

1-29. (canceled)

30. A pharmaceutical composition, comprising;
a 5-HT_{2A} receptor agonist;
nitrous oxide; and
a pharmaceutically acceptable excipient.

31. The pharmaceutical composition of claim 30, which is formulated for administration via inhalation.

32. A method of treating a subject with a central nervous system (CNS) disorder or a psychiatric disease, the method comprising:
administering to the subject a therapeutically effective amount of a 5-HT_{2A} receptor agonist and a N-methyl-D-aspartate (NMDA) receptor antagonist.

33. The method of claim 32, wherein the CNS disorder or a psychiatric disease is at least one selected from the group consisting of post-traumatic stress disorder (PTSD), major depressive disorder (MDD), treatment-resistant depression (TRD), suicidal ideation, suicidal behavior, major depressive disorder with suicidal ideation or suicidal behavior, melancholic depression, atypical depression, dysthymia, non-suicidal self-injury disorder (NSSID), bipolar and related disorders, obsessive-compulsive disorder (OCD), compulsive behavior and other related symptoms, generalized anxiety disorder (GAD), acute psychedelic crisis, social anxiety disorder, alcohol use disorder, opioid use disorder, amphetamine use disorder, nicotine use disorder, cocaine use disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, chronic fatigue syndrome, Lyme disease, gambling disorder, anorexia nervosa, bulimia nervosa, binge-eating disorder, pedophilic disorder, exhibitionistic disorder, voyeuristic disorder, fetishistic disorder, sexual masochism or sadism disorder, transvestic disorder, sexual dysfunction, peripheral neuropathy, and obesity.

34. The method of claim 32, wherein the CNS disorder or a psychiatric disease is major depressive disorder (MDD).

35. The method of claim 32, wherein the CNS disorder or a psychiatric disease is treatment-resistant depression (TRD).

36. The method of claim 32, wherein the CNS disorder or a psychiatric disease is generalized anxiety disorder (GAD).

37. The method of claim 32, wherein the CNS disorder or a psychiatric disease is social anxiety disorder.

38. The method of claim 32, wherein the CNS disorder or a psychiatric disease is obsessive-compulsive disorder (OCD).

39. The method of claim 32, wherein the CNS disorder or a psychiatric disease is alcohol use disorder.

40. The method of claim 32, wherein the 5-HT_{2A} receptor agonist is a compound of Formula (I), or a pharmaceutically acceptable salt, stereoisomer, or solvate thereof 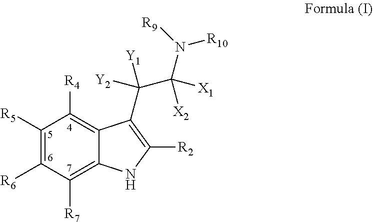
wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, and unsubstituted or substituted alkoxy;
R₆ and R₇ are independently selected from the group consisting of hydrogen, deuterium, and halogen; and
R₉ and R₁₀ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl.

41. The method of claim 32, wherein the 5-HT_{2A} receptor agonist and the NMDA receptor antagonist are administered 1 to 8 times over a treatment course.

42. The method of claim 32, wherein the 5-HT_{2A} receptor agonist and the NMDA receptor antagonist are administered concurrently as a single pharmaceutical composition.

43. The method of claim 32, wherein the 5-HT_{2A} receptor agonist and the NMDA receptor antagonist are administered as an aerosol to the subject by inhalation.

44. The method of claim 43, wherein the aerosol is in the form of a mist.

45-47. (canceled)

48. The method of claim 43, wherein the NMDA receptor antagonist is nitrous oxide.

49. The method of claim 48, wherein the aerosol comprises the nitrous oxide in a gas phase of the aerosol.

50-60. (canceled)

61. The method of claim 32, wherein the NMDA receptor antagonist is nitrous oxide or ketamine or a pharmaceutically acceptable salt, stereoisomer, or solvate thereof.

62-64. (canceled)

65. The method of claim 32, wherein the 5-HT_{2A} receptor agonist and the NMDA receptor antagonist are administered sequentially as separate pharmaceutical compositions.

66. The method of claim 32, wherein the 5-HT_{2A} receptor agonist and the NMDA receptor antagonist are administered concurrently as separate pharmaceutical compositions.

67. The method of claim 32, wherein the 5-HT_{2A} receptor agonist is administered intravenously and the NMDA receptor antagonist is administered via inhalation.

68-72. (canceled)

73. The method of claim 67, wherein the 5-HT_{2A} receptor agonist is administered to the subject intravenously as a bolus followed by a perfusion.

74. The method of claim 73, wherein a dose of the 5-HT_{2A} receptor agonist administered via the bolus and the perfusion are each independently in a range from about 0.1 mg/kg to about 0.8 mg/kg.

75-77. (canceled)

78. The method of claim 32, wherein the 5-HT_{2A} receptor agonist is N,N-dimethyltryptamine (DMT) or a pharmaceutically acceptable salt or solvate thereof.

79. The method of claim 32, wherein the 5-HT_{2A} receptor agonist is 5-methoxy-N,N-dimethyltryptamine (5-MeO-DMT) or a pharmaceutically acceptable salt or solvate thereof.

80. The method of claim 32, wherein the 5-HT_{2A} receptor agonist is 2-(1H-indol-3-yl)-N,N-bis(methyl-d₃)ethan-1-amine-1,1,2,2-d₄ (DMT-d₁₀) or a pharmaceutically acceptable salt or solvate thereof.

81. The method of claim 32, wherein the 5-HT_{2A} receptor agonist is 2-(5-methoxy-1H-indol-3-yl)-N,N-bis(methyl-d₃)ethan-1-amine-1,1,2,2-d₄ (5-MeO-DMT-d₁₀) or a pharmaceutically acceptable salt or solvate thereof.

82-93. (canceled)
